SEQUENCE LISTING (19 sequences)

SEQ ID NO: 1 - protein, 665 aa, Artificial Sequence
  homo sapiens- Fragment of human SARM1 in Fig. 2, residues 60-724 of hSARM1
EVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 2 - protein, 665 aa, Artificial Sequence
  mus musculus - Fragment of mouse SARM1 in Fig. 2, residues 60-724 of mSARM1
EVQGALERSLPELQQALSELKQASAARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALANCALHGGQTVQRCMVEKRAAEWLFPLAFSKEDELLRLHACLAVAVLATNKEVEREVEHSGTLALVEPLVASLDPGRFARCLVDASDTSQGRGPDDLQSLVLLLDSSRLEAQCIGAFYLCAEAAIKSLQGKTKVFSDIGAIQSLKRLVSYSTNGTTSALAKRALRLLGEEVPRRILPCVASWKEAEVQTWLQQIGFSQYCENFREQQVDGDLLLRLTDEELQTDLGMKSSITRKRFFRELTELKTFASYATCDRSNLADWLGSLDPRFRQYTYGLVSCGLDRSLLHRVSEQQLLEDCGIRLGVHRTRILSAAREMLHSPLPCTGGKLSGDTPDVFISYRRNSGSQLASLLKVHLQLHGFSVFIDVEKLEAGKFEDKLIQSVIAARNFVLVLSAGALDKCMQDHDCKDWVHKEIVTALSCGKNIVPIIDGFEWPEPQALPEDMQAVLTFNGIKWSHEYQEATIEKIIRFLQGRPSQDSSAGSDTSLEGATPMGLP

SEQ ID NO: 3 - protein, 661 aa, Artificial Sequence
  Danio rerio - Fragment of zebrafish SARM1 in Fig. 2, residues 60-721 of zSARM1
DVQAVLDGSLPALRSAIRTLRSSKDTGDLEETRRAIAETFQLVEEAWVLPTVGRRVAEEICNRIRLDGGLELLLQLMQTPAVEITYESAKLLEQILVSENRDYVARMGLGVILNLTREQEDAQLARSVSGILEHMFKHTEETSAQLITNGALDTILYWCRGTDPTVLRHCAVALSNCAMYGGHRCQRLMIEKQAAEWLFPLAFSKEDELIRFHACLAVAVLAANREMEKEVVKSGTLELVEPFIASLDPDEFARNMLDSADSMQGRTAADLQHLLPLLDGTRLEGKCIAAFYLCVETSIKSRQRNTKIFQEIGAVQSLKRIVMYSSNATVCSLAKRALKMMSEEVPRRILSSVPNWKSGEVQTWLQQIGFSAFSERFQELQVDGDLLLNITEQDLIQDLGMTSGLTRKRFLRDLRVLKTYANYSTCDPNNLADWLADADPRFRQYTYGLVQSGVDRNNIVHITDQQLLTDCHVENGIHRAKILSAARRPSKPCLTDSQPKGPDVFISYRRTTGSQLASLLKVHLQLRGFSVFIDVEKLEAGRFEEKLITSVQRARNFILVLSANALDKCMGDVAMKDWVHKEIVTALNGKKNIVPVTDNFVWPDPTSLPEDMSTILKFNGIKWSHEYQEATIEKILRFLEGCPSQEKPDGAKTDKKEPQKK

SEQ ID NO: 4 - protein, 673 aa, Artificial Sequence
  C. elegans - Fragment of C. elegans SARM1 in Fig. 2, residues 60-725 of ceSARM1
AMMHTLKTKLSKYQAMMDKAFEEIAKVEDANIIEGCTIVRKLMRKVWNTPKVSADLANALCDYLRDRDYFDKLIKMFISPNTAACDQVRMECGKVLEECTSSANLEYIVNKSYTKKIMIVAMKLNKTPDQQRLSLSLIGNLFKHSNAVSLSLIETDVIDHIILTFKRAPECPDILRHAALALANILLYTCFEGKKKIIQKKIPEWLFFLASQADDVTRYYACIAVCTIVSVKEFEPLVRKSDTMKLVEPFLQVHDPATFARDYHKYAQGNTKEWLERLLPMLQPSRRREARSVAAFHFTLEATIKKEQNKLDVFQEIGAIQALKEVASSPDEVAAKFASEALTVIGEEVPYKLAQQVPGWTCADVQYWVKKIGFEEYVEKFAKQMVDGDLLLQLTENDLKHDVGMISGLHRKRFLRELQTLKVAADYSSVDESNLDNFLMGLSPELSVYTYQMLTNGVNRSLLSSLTDEMMQNACGITNPIHRLKLTQAFETAKHPDDVEVAMLSKQIDVFISYRRSTGNQLASLIKVLLQLRGYRVFIDVDKLYAGKFDSSLLKNIQAAKHFILVLTPNSLDRLLNDDNCEDWVHKELKCAFEHQKNIIPIFDTAFEFPTKEDQIPNDIRMITKYNGVKWVHDYQDACMAKVVRFITGELNRTTPTTKEMPSISRKTTQQRW

SEQ ID NO: 5 - protein, 725 aa, Artificial Sequence
  N-terminal His tagged residues 26-724 of human SARM1
SYHHHHHHDYDIPTTENLYFQGAMGSERLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 6 - DNA, 2073 bp, Homo sapiens
atgggggcggtggcacgggcccatggtgggctgcgggtggcccgggcccgcgaaagtgtcgccgggggcaggcaccgaggtgcaggacgccctggagcgcgcgctgccggagctgcagcaggccttgtccgcgctgaagcaggcgggcggcgcgcgggccgtgggcgccggcctggccgaggtcttccaactggtggaggaggcctggctgctgcggccgtgggccgcgaggtagcccagggtctgtgcgacgccatccgcctcgatggcggcctcgacctgctgttgcggctgctgcaggcgccggagttggagacgcgtgtgcaggccgcgcgcctgctggagcagatcctggtggctgagaaccgagaccgcgtggcgcgcattgggctgggcgtgatcctgaacctggcgaaggaacgcgaacccgtagagctggcgcggagcgtggcaggcatcttggagcacatgttcaagcattcggaggagacatgccagaggctggtggcggccggcggcctggacgcggtgctgtattggtgccgccgcacggaccccgcgctgctgcgccactgcgcgctggcgctgggcaactgcgcgctgcacgggggccaggcggtgcagcgacgcatggtagagaagcgcgcagccgagtggctcttcccgctcgccttctccaaggaggacgagctgcttcggctgcacgcctgcctcgcagtagcggtgttggcgactaacaaggaggtggagcgcgaggtggagcgctcgggcacgctggcgctcgtggagccgcttgtggcctcgctggaccctggccgcttcgcccgctgtctggtggacgccagcgacacaagccagggccgcgggcccgacgacctgcagcgcctcgtgccgttgctcgactctaaccgcttggaggcgcagtgcatcggggctttctacctctgcgccgaggctgccatcaagagcctgcaaggcaagaccaaggtgttcagcgacatcggcgccatccagagcctgaaacgcctggtttcctactctaccaatggcactaagtcggcgctggccaagcgcgcgctgcgcctgctgggcgaggaggtgccacggcccatcctgccctccgtgcccagctggaaggaggccgaggttcagacgtggctgcagcagatcggtttctccaagtactgcgagagcttccgggagcagcaggtggatggcgacctgcttctgcggctcacggaggaggaactccagaccgacctgggcatgaaatcgggcatcacccgcaagaggttctttagggagctcacggagctcaagaccttcgccaactattctacgtgcgaccgcagcaacctggcggactggctgggcagcctggacccgcgcttccgtcagtacacctacggcctggtcagctgcggcctggaccgctccctgctgcaccgcgtgtctgagcagcagctgctggaagactgcggcatccacctgggcgtgcaccgcgcccgcatcctcacggcggccagagaaatgctacactccccgctgccctgtactggtggcaaacccagtggggacactccagatgtcttcatcagctaccgccggaactcaggttcccagctggccagtctcctgaaggtgcacctgcagctgcatggcttcagtgtcttcattgatgtggagaagctggaagcaggcaagttcgaggacaaactcatccagagtgtcatgggtgcccgcaactttgtgttggtgctatcacctggagcactggacaagtgcatgcaagaccatgactgcaaggattgggtgcataaggagattgtgactgctttaagctgcggcaagaacattgtgcccatcattgatggcttcgagtggcctgagccccaggtcctgcctgaggacatgcaggctgtgcttactttcaacggtatcaagtggtcccacgaataccaggaggccaccattgagaagatcatccgcttcctgcagggccgctcctcccgggactcatctgcaggctctgacaccagtttggagggtgctgcacccatgggtccaacctaa

SEQ ID NO: 7 - protein, 245 aa, Artificial Sequence
  ARM1 domain of Human SARM1 Residues 60-404
EVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLD

SEQ ID NO: 8 - protein, 101 aa, Artificial Sequence
  ARM2 domain of Human SARM1 Residues 305-400
PGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPI

SEQ ID NO: 9 - protein, 74 aa, Artificial Sequence
  SAM1 domain of Human SARM1 -Residues 406-479
LPSVPSWKEAEVQTWLQQIGFSKYCESFREQQVDGDLLLRLTEEELQTDLGMKSGITRKRFFRELTELKTFANY

SEQ ID NO: 10 - protein, 78 aa, Artificial Sequence
  SAM2 domain of Human SARM1 -Residues 480-551
STCDRSNLADWLGSLDPRFRQYTYGLVSCGLDRSLLHRVSEQQLLEDCGIHLGVHRARILTAAREMLHSPLPCTGGKP

SEQ ID NO: 11 - protein, 144 aa, Artificial Sequence
  TIR domain of Human SARM1 Residues 557-701
SGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQG

SEQ ID NO: 12 - protein, 724 aa, Homo sapiens
MVLTLLLSAYKLCRFFAMSGPRPGAERLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 13 - protein, 699 aa, Artificial Sequence
  HUMAN_sp|Q6SZW1|SARM1_HUMAN, residues 26 to 724 of hSARM1
ERLAVPGPDGGGGTGPWWAAGGRGPREVSPGAGTEVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQGRSSRDSSAGSDTSLEGAAPMGPT

SEQ ID NO: 14 - protein, 642 aa, Artificial Sequence
  TIR and ARM domains (residue 60 to 701 of the human wild type SARM1)
EVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEEVPRPILPSVPSWKEAEVQTWLQQIGFSKYCESFREQQVDGDLLLRLTEEELQTDLGMKSGITRKRFFRELTELKTFANYSTCDRSNLADWLGSLDPRFRQYTYGLVSCGLDRSLLHRVSEQQLLEDCGIHLGVHRARILTAAREMLHSPLPCTGGKPSGDTPDVFISYRRNSGSQLASLLKVHLQLHGFSVFIDVEKLEAGKFEDKLIQSVMGARNFVLVLSPGALDKCMQDHDCKDWVHKEIVTALSCGKNIVPIIDGFEWPEPQVLPEDMQAVLTFNGIKWSHEYQEATIEKIIRFLQG

SEQ ID NO: 15 - protein, 244 aa, Artificial Sequence
  ARM-1 residues 60-303, of the human wild type SARM1
EVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASL

SEQ ID NO: 16 - protein, 341 aa, Artificial Sequence
  seven three-helix ARM repeats spanning residues 60-400 of SARM1
EVQDALERALPELQQALSALKQAGGARAVGAGLAEVFQLVEEAWLLPAVGREVAQGLCDAIRLDGGLDLLLRLLQAPELETRVQAARLLEQILVAENRDRVARIGLGVILNLAKEREPVELARSVAGILEHMFKHSEETCQRLVAAGGLDAVLYWCRRTDPALLRHCALALGNCALHGGQAVQRRMVEKRAAEWLFPLAFSKEDELLRLHACLAVAVLATNKEVEREVERSGTLALVEPLVASLDPGRFARCLVDASDTSQGRGPDDLQRLVPLLDSNRLEAQCIGAFYLCAEAAIKSLQGKTKVFSDIGAIQSLKRLVSYSTNGTKSALAKRALRLLGEE

SEQ ID NO: 17 - protein, 79 aa, Artificial Sequence
  ARM2 residues 322 to 400, of the human SARM1
RGPDDLQRLVPLLDSNRLEAQCIGAFYLCAEAAIKSLQGKTKVFSDIGAIQSLKRLVSYSTNGTKSALAKRALRLLGEE

SEQ ID NO: 18 - protein, 16 aa, Artificial Sequence
  ARM1 alpha 2- alpha 3 loop with the loop that connects ARM1 with ARM2 (residues 305-320 of the human SARM1
PGRFARCLVDASDTSQ

SEQ ID NO: 19 - protein, 5 aa, Artificial Sequence
  ARM -SAM1 connected via a linker, residues 400-404 of the human SARM1
VPRPI

(19) United States
(12) Patent Application Publication
(10) Pub. No.: US 2022/0241416 A1
(43) Pub. Date: Aug. 4, 2022

(54) METHODS FOR THE IDENTIFICATION OF SARM1 NADASE ACTIVITY INHIBITORS AND USES THEREOF

(71) Applicant: Bar Ilan University, Ramat Gan, FL (US)

(72) Inventor: Yarden OPATOWSKY, Raanana (IL)

(21) Appl. No.: 17/586,245

(22) Filed: Jan. 27, 2022

Related U.S. Application Data
(60) Provisional application No. 63/142,650, filed on Jan. 28, 2021.

(51) Int. Cl. A61K 41/00; A61P 25/00; G01N 21/64

(52) U.S. Cl. CPC A61K 41/00; G01N 21/6428; A61P 25/00

(57) ABSTRACT

The present disclosure provides screening methods for identifying a sterile α and HEAT/armadillo motif-containing protein-1 (SARM1) NADase modulator, specifically, inhibitor, using at least one full length or near full length catalytically active SARM1 or any fragment or variant thereof. The present disclosure further provides SARM inhibitors identified by the screening methods, and therapeutic compositions and methods for treating, conditions or disorders associated directly or indirectly with axonal and/or cellular degradation.

### TECHNOLOGICAL FIELD

The present invention relates to screening methods and kits for identification of sterile a and HEAT/armadillo motif-containing protein-1 (SARM1) inhibitors and their use for treating/preventing conditions and pathological disorders associated with axonal and/or cellular degeneration and/or degradation.

### BACKGROUND ART

References considered to be relevant as background to the presently disclosed subject matter are listed below:
[1] J. M. Osterloh et al., dSarm/Sarm1 is required for activation of an injury-induced axon death pathway. Science 337, 481-484 (2012).
[2] J. Gerdts, D. W. Summers, Y. Sasaki, A. DiAntonio, J. Milbrandt. Sarm1-mediated axon degeneration requires both SAM and TIR interactions. J Neurosci 33, 13569-13580 (2013).
[3] M. B. Uccellini et al., Passenger Mutations Confound Phenotypes of SARM1-Deficient Mice. Cell reports 31, 107498 (2020).
[4] Y. Kim et al., MyD88-5 links mitochondria, microtubules, and JNK3 in neurons and regulates neuronal survival. J Exp Med 204, 2063-2074 (2007).
[5] E. Ozaki et al., SARM1 deficiency promotes rod and cone photoreceptor cell survival in a model of retinal degeneration. Life Sci Alliance 3, (2020).
[6] K. W. Ko, J. Milbrandt, A. DiAntonio, SARM1 acts downstream of neuroinflammatory and necroptotic signaling to induce axon degeneration. J Cell Biol 219, (2020).
[7] R. Krauss, T. Bosanac, R. Devraj, T. Engber, R. O. Hughes, Axons Matter: The Promise of Treating Neurodegenerative Disorders by Targeting SARM1-Mediated Axonal Degeneration. Trends Pharmacol Sci 41, 281-293 (2020).
[8] P. Panneerselvam, L. P. Singh, B. Ho, J. Chen, J. L. Ding, Targeting of pro-apoptotic TLR adaptor SARM to mitochondria: definition of the critical region and residues in the signal sequence. Biochem J 442, 263-271 (2012).
[9] D. W. Summers, D. A. Gibson, A. DiAntonio, J. Milbrandt. SARM1-specific motifs in the TIR domain enable NAD+ loss and regulate injury-induced SARM1 activation. Proc Natl Acad Sci USA 113, E6271-E6280 (2016).
[10] C. F. Chuang, C. I. Bargmann, A Toll-interleukin 1 repeat protein at the synapse specifies asymmetric odorant receptor expression via ASK1 MAPKKK signaling. Genes Dev 19, 270-281 (2005).
[11] J. Gerdts, E. J. Brace, Y. Sasaki, A. Di Antonio, J. Milbrandt, SARM1 activation triggers axon degeneration locally via NAD(+) destruction. Science 348, 453-457 (2015).
[12] S. Horsefield et al., NAD(+) cleavage activity by animal and plant TIR domains in cell death pathways. Science 365, 793-799 (2019).
[13] K. Essuman et al., The SARM1 Toll/Interleukin-1 Receptor Domain Possesses Intrinsic NAD(+) Cleavage Activity that Promotes Pathological Axonal Degeneration. Neuron 93, 1334-1343 e1335 (2017).
[14] S. Geisler et al., Gene therapy targeting SARM1 blocks pathological axon degeneration in mice. J Exp Med 216, 294-303 (2019).
[15] J. Gerdts, D. W. Summers, J. Milbrandt, A. Di Antonio, Axon Self-Destruction: New Links among SARM, MAPKs, and NAD+ Metabolism. Neuron 89, 449-460 (2016).
[16] M. Spomy et al., Structural Evidence for an Octameric Ring Arrangement of SARM1. J Mol Biol 431, 3591-3605 (2019).
[17] H. Murata, M. Sakaguchi, K. Kataoka, N. H. Huh, SARM1 and TRAF6 bind to and stabilize PINK1 on depolarized mitochondria. Mol Biol Cell 24, 2772-2784 (2013).
[18] D. W. Summers, A. DiAntonio, J. Milbrandt, Mitochondrial dysfunction induces Sarm1-dependent cell death in sensory neurons. J Neurosci 34, 9338-9350 (2014).
[19] J. Gilley, G. Orsomando, I. Nascimento-Ferreira, M. P. Coleman, Absence of SARM1 rescues development and survival of NMNAT2-deficient axons. Cell reports 10, 1974-1981 (2015).
[20] S. Geisler et al., Prevention of vincristine-induced peripheral neuropathy by genetic deletion of SARM1 in mice. Brain 139, 3092-3108 (2016).
[21] Z. Y. Zhao et al., A Cell-Permeant Mimetic of NMN Activates SARM1 to Produce Cyclic ADP-Ribose and Induce Non-apoptotic Cell Death. iScience 15, 452-466 (2019).
[22] H. W. Liu et al., Pharmacological bypass of NAD(+) salvage pathway protects neurons from chemotherapy-induced degeneration. Proc Natl Acad Sci USA 115, 10654-10659 (2018).
[23] H. Murata et al., c-Jun N-terminal kinase (JNK)-mediated phosphorylation of SARM1 regulates NAD(+) cleavage activity to inhibit mitochondrial respiration. J Biol Chem 293, 18933-18943 (2018).
[24] US 2014/007972 A1.
[25] WO 208/057989.
[26] R. O. Hughes et al., Cell Reports 34 (5/1/021) https://doi.org/10.1016/j.celrep.2020.108588
[27] M. Sporny, et al., The Structural Basis for SARM1 Inhibition, and Activation Under Energetic Stress bioRxiv, August 2020, https://elifesciences.org/articles/62021.
[28] Y. Jiang, The NAD⁺-mediated self-inhibition mechanism of pro-neurodegenerative Sarm1. Nature https://doi.org/10.1038/s41586-020-2862-z, (2020).
[29] K. Essuman et al., TIR Domain Proteins Are an Ancient Family of NAD(+)-Consuming Enzymes. Curr Biol 28, 421-430 e424 (2018).
[30] M. D. Figley, A. DiAntonio, The SARM1 axon degeneration pathway: control of the NAD(+) metabolome regulates axon survival in health and disease. Current opinion in neurobiology 63, 59-66 (2020).
[31] Y. Sasaki, T. Nakagawa, X. Mao, A. DiAntonio, J. Milbrandt, NMNAT1 inhibits axon degeneration via blockade of SARM1-mediated NAD(+) depletion. Elife 5, (2016). Acknowledgement of the above references herein is not to be inferred as meaning that these are in any way relevant to the patentability of the presently disclosed subject matter.

### BACKGROUND

SARM (sterile α and HEAT/armadillo motif-containing protein) was first discovered as a negative regulator of TRIF (TIR domain-containing adaptor inducing interferon-β) in TLR (Toll-like receptor) signaling and was later shown to promote neuronal death by oxygen and glucose deprivation and viral, bacterial and fungal infections. Multiple studies have demonstrated that SARM1 is a key part of a highly conserved axonal death pathway that is activated by nerve injury [1-2]. Importantly, recent studies have shown that SARM1 deficiency confers protection against axonal degeneration in several models of neurodegenerative conditions [3-6], making SARM1 a compelling molecular target for the development of safe and effective pharmacological therapy to protect axons in a variety of axonopathies [7]. The domain composition of SARM1 includes an N-terminal peptide, an ARM-repeats region, two SAM and one TIR domain (FIG. 1A, FIG. 2), which mediate mitochondria targeting [8], auto-inhibition [9-10], oligomerization [2], and NAD⁺-glycohydrolase (NADase) activity [1], respectively. Amino acid substitution (E642A) at the TIR domain's active site [2] abolishes the NADase activity in vitro and inactivates SARM1 pro-degenerative activity [13-14], thereby linking the role of SARM1 in axonal degeneration with its NADase activity. The enzymatic activity requires a high local concentration of the TIR domains, as demonstrated by forced dimerization of TIR, which resulted in NAD+ hydrolysis and neuronal cell death [11, 15]. Also, in the absence of the auto-inhibitory ARM domain, which can interact directly with TIR [9], the remaining SAM-TIR construct is active and leads to rapid cell death [2, 16]. The mechanism by which SAM domains cause TIR crowding became clearer in a previous report, where it was showed that both hSARM1 and the isolated tandem SAM1-2 domains form octamers in solution [6]. Also, negative stain electron microscopy analysis of hSARM1 was used and the crystal structure of the SAM1-2 domains was determined—both of which revealed an octameric ring arrangement. Based on these findings, it appears that hSARM1 is kept auto-inhibited by the ARM domain in homeostasis, and gains NADase activity upon the infliction of injury and other stress conditions (axotomy [1], oxidative (mitochondria depolarization [7]; oxidizing agents [18], metabolic (depletion of NAD+[9]), or toxic (chemotherapy drugs [20]). Whether and how all or some of these insults converge to induce SARM1 activation is still not completely understood. In this regard, little is known about the direct molecular triggers of SARM1 in cells, besides the potential involvement of nicotinamide mononucleotide (NMN) [21-22] and Ser-548 phosphorylation by JNK [23] in promoting the NADase activity of SARM1. US 2014/007972 [24] discloses the characterization of mammalian SARM1 in promoting axonal destruction, showing that loss of SARM function suppresses neuronal damage. WO 208/057989 [25] provides several compounds directed at the TIR domain of SARM1, as effective inhibitors of SARM1 NADase activity. This publication further provides methods for identifying inhibitors SARM1 NADase activity, by contacting candidate compounds with the SAM-TIR-domains of SARM1, specifically with SARM1 residues 410-721, that is devoid of the N-terminal auto-inhibitory domain. These publications however target the catalytic domain of SARM1. Similarly, an additional recent publication by Hughes et al., [26], reported the use of the TIR domain in high-throughput screen of small molecule compounds. The assay is based on biochemical cell-lysate assay in which a recombinant construct of the constitutively active SAM-TIR protein of human SARM1 was overexpressed in mammalian cells. Using this system, the production of ADPR and the hydrolysis of NAD+ was measured by rapid-fire mass spectrometry. Isoquinoline identified by this screen demonstrated protection of axons degradation.

Still further, the present inventors [27]) and another more recent study [28] showed that NAD+ is a ligand of the ARM domain and facilitates inhibition of the TIR-domain NADase by ARM. There is a need in understanding the molecular basis of SARM1 inhibition and its activation under stress conditions and to develop effective direct or allosteric SARM1 inhibitors and open the door for the development of novel and potent drugs for preventing exonal and/or synaptic degradation, and conditions associated therewith.

### SUMMARY OF THE INVENTION

In a first aspect, the present disclosure relates to a screening method for identifying a modulator, and specifically, inhibitor of sterile a and HEAT/armadillo motif-containing protein-1 (SARM1) NADase. In some specific embodiments, the method comprising the following steps:

First (a), incubating (i) at least one candidate compound; and (ii) at least one full length, or nearly full length catalytically active SARM1 or any fragment or variant thereof, or any mixture comprising (1) and (ii). In some embodiments, any fragment or variant of SARM1 that may be useful in the methods of the present disclosure comprises the TIR domain and the TIR docking site of the ARM-1 domain.

The next step (b), involves adding to the incubated candidate and SARM1, or mixture of (a), at least one SARM1 substrate or any analogues thereof, and incubating the mixture under suitable conditions.

In the next step (c), quantifying the amount of the SARM1 substrate or of any hydrolysis products thereof in the incubated mixture of (b); by a suitable means.

The final step (d) involves identifying the candidate compound as an inhibitor of SARM1 NADase. More specifically, a candidate compound is identified as an inhibitor of SARM1 NADase if step (c) of the method of the invention, results in at least one of the following outcomes in the presence of the candidate.

In some embodiments, a candidate compound is considered as an inhibitor if (i), the amount of the SARM1 substrate in the mixture of (c) is not reduced over time. It should be noted that since NAD+ is SARM substrate, reduction of the SARM1 substrate amount over the reaction time reflects SARM1 NADase activity. A candidate compound that attenuates or inhibits the reduction of the SARM1 substrate amount, may be considered as inhibiting SARM1 NADase activity. In another alternative or additional embodiment (ii), a candidate compound may be considered as an inhibitor of SARM1 NADase activity, if the amount of the SARM1 substrate in the mixture of (c), is greater than that of a control mixture that does not contain the candidate compound. In some embodiments, the SARM1 substrate is Nicotinamide adenine dinucleotide+(NAD+), or any analogues thereof.

A further aspect of the present disclosure relates to a kit or system comprising:

(a) at least one near full length catalytically active SARM1 protein or variant thereof, or any nucleic acid sequence encoding the SARM1 protein.

Still further, the kit of the invention may comprise (b), at least one reagent and/or material for quantifying the amount of SARM1 substrate or of any hydrolysis products thereof, and/or for assessing/determining the conformation of the SARM1 provided by the kit.

The kit of the invention further comprises (c), at least one SARM1 substrate or any analogues thereof. In some embodiments, the SARM1 substrate is Nicotinamide adenine dinucleotide+(NAD+), or any analogues thereof.

A further aspect of the present disclosure relates to a SARM1 NADase inhibitor obtained by a screening method. In some embodiments, the inhibitor of the invention is identified by a method comprising the steps of:

First (a), incubating (i) at least one candidate compound: and (ii) at least one near full length catalytically active SARM1 or any fragment or variant thereof, or any mixture comprising (i) and (ii). In some embodiments, any fragment or variant of SARM1 that may be useful in the methods of the present disclosure comprises the TIR domain and the TIR docking site of the ARM-1 domain.

The next step (b), involves adding to the mixture of (a), at least one SARM1 substrate or any analogues thereof, and incubating the mixture under suitable conditions.

In the next step (c), quantifying the amount of SARM1 substrate or of any hydrolysis products thereof in the incubated mixture of (b); by a suitable means.

The final step (d), involves identifying the candidate compound as an inhibitor of SARM1 NADase, if step (c) in the presence of the candidate results in at least one of:

(i) if the amount of said SARM1 substrate in the mixture of (c) is not reduced over time;
(ii) if the amount of SARM1 substrate in the mixture of (c) is greater than that of a control mixture that does not contain said candidate compound; and/or (ii) if the SARM1 in the mixture of (c) displays a two-ring compact conformation. In some embodiments, the SARM1 substrate is Nicotinamide adenine dinucleotide+(NAD+), or any analogues thereof.

A further aspect of the present disclosure relates to a composition comprising as an active ingredient an effective amount of at least one SARM1 NADase inhibitor as by the invention, or any nano- or micro-particle, micellar formulation, vehicle or matrix comprising said inhibitor, said composition optionally further comprises at least one of pharmaceutically acceptable carrier/s, excipient/s, auxiliaries, and/or diluents.

In yet a further aspect, the present disclosure provides a method for treating, preventing, inhibiting, reducing, eliminating, protecting or delaying the onset of conditions or disorders associated directly or indirectly with axonal and/or cellular degradation in a subject in need thereof. The method comprises in some embodiments the step of administering to the subject a therapeutically effective amount of at least one SARM1 NADase inhibitor as defined by the present disclosure, or any nano- or micro-particle, micellar formulation, vehicle, matrix or composition comprising said inhibitor. In more specific embodiments, the inhibitor is obtained by a screening method comprising the steps of: (a). incubating (i) at least one candidate compound; and (ii) at least one full length or near full length catalytically active SARM1 or any fragment or variant thereof, or any mixture thereof. The fragment or variant of SARM1 comprises the TIR domain and the TIR docking site of the ARM-1 domain. Th next step (b). adding to the mixture of (a), at least one SARM1 substrate or any analogues thereof, and incubating said mixture under suitable conditions. In the next step (c), quantifying the amount of said SARM1 substrate or of any hydrolysis products thereof in the incubated mixture of (b); and/or assessing and/or determining the conformation of said SARM1 in the incubated mixture of (b) by a suitable means; and (d). identifying said candidate compound as an inhibitor of SARM1 NADase if at least one of:

(i) the amount of said SARM1 substrate in the mixture of (c) is not reduced over time;
(ii) the amount of SARM1 substrate in the mixture of (c) is greater than that of a control mixture that does not contain said candidate compound; and (iii) the SARM1 in the mixture of (c) displays a two-ring compact conformation.

A further aspect of the present disclosure relates to a method for treating, preventing, inhibiting, reducing, eliminating, protecting or delaying the onset of conditions or disorders associated directly or indirectly with axonal and/or cellular degradation in a subject in need thereof. The method comprises in some embodiments the step of:

(I) screening for said inhibitor, comprising:
(a). incubating (i) at least one candidate compound; and (ii) at least one full length or near full length catalytically active SARM1 or any fragment or variant thereof, or any mixture thereof, wherein said fragment or variant of SARM1 comprises the TIR domain and the TIR docking site of the ARM-1 domain; (b). adding to the mixture of (a), at least one SARM1 substrate or any analogues thereof, and incubating said mixture under suitable conditions; (c). quantifying the amount of said SARM1 substrate or of any hydrolysis products thereof in the incubated mixture of (b); and/or assessing and/or determining the conformation of said SARM1 in the incubated mixture of (b) by a suitable means; and
(d). identifying said candidate compound as an inhibitor of SARM1 NADase if at least one of: (i) the amount of said SARM1 substrate in the mixture of (c) is not reduced over time; (ii) the amount of SARM1 substrate in the mixture of (c) is greater than that of a control mixture that does not contain said candidate compound; and (iii) the SARM1 in the mixture of (c) displays a two-ring compact conformation; and
(II) administering to the subject a therapeutically effective amount of at least one SARM1 NADase inhibitor obtained in step (I), or any nano- or micro-particle, micellar formulation, vehicle, matrix or composition comprising the inhibitor.

A further aspect of the invention relates to a method for inhibiting SARM1 NADase activity in a cell. More specifically, the method comprises the step of contacting the cell with an effective amount of at least one SARM1 NADase inhibitor as defined by the present disclosure, or any nano- or micro-particle, micellar formulation, vehicle, matrix or composition comprising said inhibitor.

These and other aspects of the present disclosure will become apparent by the hand of the following examples and description.

### BRIEF DESCRIPTION OF THE DRAWINGS

The patent or application file contains at least one drawing executed in color. Copies of this patent or patent application publication with color drawings will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1A-1D: Domain organization and cryo-EM analysis of purified hSARM1

FIG. 1A: Color-coded organization and nomenclature of the SARM1 ARM, SAM, and TIR domains. The position of mitochondria) localization signal is presented at the N′ terminus of the protein. Two constructs are used in this study, both missing the mitochondria) N′ terminal sequence. hSARM1E642Q is a NADase attenuated mutant that was used in all the structural and some of the biochemical experiments; while hSARM1 w.t. was used in the NADase and cellular experiments.

FIG. 1B: hSARM1 protein preparations. Presented are SDS-PAGE analyses of size-exclusion chromatography fractions after the initial metal-chelate chromatography step. Note the higher yield of hSARM1E642Q compared to hSARM1w.t. The white arrow indicates the fraction used for the subsequent GraFix step shown at the right panel, where the approximate glycerol concentrations of each fraction are indicated.

FIG. 1C: Most-prevalent 2D class averages of hSARM1E642Q protein preparations. A dramatic difference can be seen between the previously-conducted negative stain analysis (top panel, as in [6]) and typical cryo-EM (middle panel). While the negative stain average clearly shows inner and peripheral rings (see illustration in the bottom panel), most cryo-EM classes depict only the inner ring, and some a partial outer ring assembly. Note that a gradient fixation (GraFix) protocol was applied before the negative stain but not the cryo-EM measurements.

FIG. 1D: 3D cryo-EM reconstructions of hSARM1E642Q (top panel) and SAM1-2 (bottom panel) and docking of the SAM1-2 crystal structure (PDB code 6QWV) into the density maps, further demonstrating that only the inner. SAM1-2 ring is well-ordered in cryo-EM of purified hSARM1E642Q.

FIG. 2 (and 2-1): SARM1 Structure-based sequence alignment

Structure-based sequence alignment of the SARM1 of human, mouse, zebrafish and the C. elegans homolog TIR-1, as denoted by SEQ ID NO: 1, 2, 3 and 4 respectively. Color coded highlights and Uniprot protein accession numbers are listed below.

FIG. 3A-3D: Cryo-EM structure of GraFix-ed hSARM1E642Q. Related to supplementary figures S2A and S3A

FIG. 3A: Selected representation of 2D class averages used for the 3D reconstruction. The number of particles that were included in each average are indicated at the top of each class.

FIG. 3B-3D: Cryo-EM density map color-coded as in (FIG. 1A) and by chain. ‘Top view’ refers to the aspect of the molecule showing the TIR (red) and SAM2 (cyan) domains closest to the viewer, while in the ‘bottom view’ the SAM1 (blue) domains and the illustrated mitochondria) N′-terminal localization tag (green dot—was not included in the expression construct) are the closest. FIG. 3D is a side view representation of the structure, sliced at the frontal plane of the aspect presented in FIG. 3B and FIG. 3C.

FIG. 4A-4B: Illustration of NAD+ supplemented hSARM1 FIG. 4A: Cartoon model of the NAD+ supplemented hSARM1. Color code is as in FIG. 1A.

FIG. 4B: Architecture of the crescent-shaped ARM domain. The structure reveals that there are two ARM subdomains, one spanning res. 60-303 with five 3-helix (depicted as green, yellow and blue cylinders) ARM repeats, and the second (res. 322-400) with two repeats, all colored in orange. The SAM and TIR domains are represented as transparent blue and red surface, respectively.

FIG. 5A-5B: hSARM1 Cryo-EM maps

The figure shows Resolution, angular distribution, and B-factor estimations of the Cryo-EM maps of GraFix-ed (FIG. 5A) and NAD+ supplemented (FIG. 5B) hSARM1

FIG. 6A-6F: Structural basis for hSARM1 auto-inhibition

FIG. 6A: Close-up of a tilted side view of the GraFix-ed hSARM1E642Q map (colored as in FIG. 3A). Two neighboring ARM domains (yellow) are outlined by a black line and the NAD+ binding cleft of a TIR domain that is bound to the two ARMs is highlighted in green. The interfaces formed between the TIR and ARMs are designated as the ‘primary TIR docking site’ and the ‘secondary TIR docking site’.

FIG. 6B: An ‘open-book’ representation of the ‘primary TIR docking site’. Left—the TIR and ARM domains are colored as in FIG. 6A, and the interface surfaces in gray are encircled by green dashed line. Site-directed mutagenesis sites are indicated. Right—amino acid conservation at the ‘primary TIR docking site’. Cyan through maroon are used to indicate amino acids, from variable to conserved, demonstrating an overall high level of conservation in this interface.

FIG. 6C: Toxicity of the hSARM1 construct and mutants in HEK293T cells. The cells were transfected with hSARM1 expression vectors, as indicated. Cell viability was measured and quantified 24 h post-transfection using the fluorescent resazurin assay. While cell viability is virtually unaffected after 24 hr by ectopic expression of hSARM1 w.t. and hSARM1E642Q, deletion of the inhibiting ARM domain (which results in the SAM1-2-TIR construct) induces massive cell death. Mutations at the ‘primary TIR docking site’ of the ARM domain also induce cell death, similar to the ‘delARM’ construct (three biological repeats, Student t test: *** p <0.001; * p <0.05; n.s: no significance).

FIG. 6D: Kinetic measurement of purified hSARM1 NADase activity. Km and Vmax were determined by fitting the data to the Michaelis-Menten equation and are presented as mean±SEM for three independent measurements.

FIG. 6E: HPLC analysis of time dependent NAD+ consumption (50 μM) by hSARM1, and further activation by NMN (0.2 and 1 mM). Time points 0 (black), 5 (green), and 30 (red) minutes. Right graph shows the rate of ADPR product generation: no NMN (green). 0.2 mM NMN (orange), 1 mM NMN (red). While 0.2 mM NMN has no visible effect, 1 mM NMN increases hSARM1 activity by ˜30%.

FIG. 6F: Glycerol inhibition of hSARM1 NADase in vitro activity was measured 20 minutes after adding 0.5 μM NAD+ to the reaction mixture. In the same way, hSARM1E642Q activity was measured and compared to that of hSARM1w.t., showing attenuated NADase activity of the former.

FIG. 7A-7E: NAD+ induces structural and enzymatic inhibition of hSARM1

FIG. 7A: Inhibition of hSARM1 NADase activity by ATP was demonstrated and analyzed as in FIG. 6F.

FIG. 7B: The structural effects of NAD+ and ATP were observed by cryo-EM based on appearance in 2D classification. Presented are the 10 most populated classes (those with the largest number of particles—numbers in green, 1st class is on the left) out of 50-100 from each data collection after the first round of particle picking and classification. By this analysis, the percentage of particles that present full, two-ring structure is 13% (no NAD+); 74% (5 mM NAD+); and 4% (10 mM ATP).

FIG. 7C: Quantification of the number of particles with full ring assembly vs. those where only the inner ring is visible from a total number of ˜0.5 million particles in each dataset. Conditions of sample preparation, freezing, collection, and processing were identical, except for the NAD+ supplement in one of the samples.

FIG. 7D: Rate of change in nucleotide fluorescence under steady-state conditions of eNAD hydrolysis by hSARM1. Reactions were initiated by mixing 400 nM enzyme with different concentrations of 1:10 eNAD+: NAD+(mol/mol) mixtures. Three repeats, standard deviation error bars.

FIG. 7E: HPLC analysis of time dependent NAD+ consumption by hSARM1 and porcine brain NADase control in 50 μM and 2 mM. Time points 0 (black), 5 (green), and 30 (red) minutes. Inset graph shows the rate of ADPR product generation. While the rate of NAD+ hydrolysis by porcine NADase is maintained through 50 μM and 2 mM NAD+, hSARM1 is tightly inhibited by 2 mM NAD+.

FIG. 8A-8D: 3D structure reveals an inhibitory ARM allosteric NAD+ binding site

FIG. 8A: Selected representation of 2D class averages used for the 3D reconstruction of hSARM1E642Q supplemented with 5 mM of NAD+. The number of particles that were included in each class average are indicated.

FIG. 8B: Related to FIG. 5B. Color coded (as in FIG. 1A) protein model docked in a transparent 2.7 Å cryo-EM density map (gray).

FIG. 8C: When compared to the GraFix-ed map, without NAD+ supplement (left), an extra density appears at the ‘ARM horns’ region in the NAD+ supplemented map (middle, right). The extra density is rendered in green and an NAD+ molecule is fitted to it. The NAD+ is surrounded by three structural elements, as indicated on the right panel. The NAD+ directly interacts with the surrounding residues: the nicotinamide moiety is stacked onto the W103 sidechain rings; the following ribose with L152; R157 and K193 form salt bridges with phosphate alpha (distal from the nicotinamide). The map density at the distal ribose and adenosine moieties is less sharp, but clearly involves interactions with the ARM2 R322. G323 and D326. In this way, activation by NMN, that lacks the distal phosphate, ribose and adenosine, can be explained by binding to ARM1 while preventing the bridging interactions with ARM2.

FIG. 8D: Toxicity of the hSARM1 construct and mutants in HEK293F cells. The cells were transfected with hSARM1 expression vectors, as indicated. Viable cells were counted 48 (bars inn gray) and 72 (bars in black stripes) hours post-transfection. Moderate reduction in cell viability due to ectopic expression of hSARM1 w.t. becomes apparent 72 hours after transfection, when compared with the NADase attenuated hSARM1E642Q, while the ‘delARM’ construct marks a constitutive activity that brings about almost complete cell death after three days. Mutations at the ARM1 α5-6 and ARM1-ARM2 loops induce cell death at a similar level as the ‘deIARM’ construct, while the control mutations and W103A did not show increased activity (three biological repeats, Student t test; *** p <0.001; * p <0.05; n.s: no significance).

FIG. 9A-9C: Cartoon model and density map of the NAD+ supplemented hSARM1

FIG. 9A: A zoom-in bottom view with indication of the ‘ARM horns’ region. Color code is as in FIG. 1 A. NAD+ densities are in green. Note the ARM domain interactions with neighboring SAM and ARM domains.

FIG. 9B: A zoom-in view of the TIR domain docking onto two neighboring ARM domains. The NAD+ binding cleft and BB loop are indicated.

FIG. 9C: Density map (in transparent gray) showing a close-up view of isolated segments of the ARM. SAM and TIR sections. The backbone and side chains are represented as sticks and colored in yellow, blue, and red, respectively, as in FIG. 1A.

FIG. 10: A model for hSARM1 inhibition and activation

The figure illustrates the inhibitory two-ring conformation and the active one-ring conformation of SARM1. In the inhibitory conformation above, the catalytic TIR domains (red) are docked on ARM domains (yellow) apart from each other, unable to form close dimers required for NAD+ catalysis. When cellular NAD+ levels drop, the inhibiting NAD+ molecules fall off hSARM1, leading to the disintegration of the ARM-TIR outer ring assembly. Still held by the constitutively-assembled SAM inner ring, the now-released TIR domains dimerize, thereby forming an active conformation.

FIG. 11A-11E: Optimization of resazurin florescence assay for high throughput screening

FIG. 11A. First, NAD+ is pre-incubated with hSARM1 along with each one of the screened compounds. Depending on the NADase activity of hSARM1 in the presence of a particular compound, NAD+ will be consumed, e.g. a complete inhibition of hSARM1 will leave all the NAD+ intact. Next, the underscored components of the enzymatic coupled reaction are added, and eventually a fluorescent signal is gained. High fluorescence indicates for high NAD+ levels and hSARM1 inhibition, and low fluorescence for hSARM1 free of inhibition. If reaction quenching is required, the alcohol dehydrogenase inhibitor iodoacetamide will be added.

FIG. 11B. Progression of fluorescent signal (measurement every 5 minutes for an hour. Data are presented as mean±SEM for sixteen repeats) over time in response to various NAD+ inputs. The assay distinguishes between NAD+ levels in the 1-250 nM range up to 25 minutes after reaction commencement.

FIG. 11C. Iodoacetamide inhibits the activity of alcohol dehydrogenase, and so fixes the fluorescence output signal for >100 minutes. Data are presented as mean±SEM for four repeats.

FIG. 11D. Pre-incubation of NAD+ with hSARM1 demonstrates a hSARM1 dose response on fluorescence output signal. Data are presented as mean±SEM for four repeats.

FIG. 11E. Fluorescence output responds to hSARM1 inhibition by 1 mM of NAM (pre-incubation for 10 minutes at room temperature). Data are presented as mean±SEM for four repeats.

FIG. 12A-12D: Potency and mode of inhibition of hSARM1 by the chemical compounds

FIG. 12A. Chemical structure of hSARM1 inhibitory compounds TK138, as denoted by Formula I, and TK142, as denoted by Formula II.

FIG. 12B. Determination of IC₅₀ values. Various concentrations of the compounds were pre-incubated with hSARM1 for 10 minutes at room temperature, followed by addition of 50 μM NAD+, initiating the NADase activity. All the assays were carried out at 37° C. for 10 minutes. For reference, the IC₅₀ values of the compounds were compared to nicotinamide (NAM), a known hSARM1 inhibitor (see overlaid on the TK142 plot).

FIG. 12C. Lineweaver-Burk plots for determination of the mode of inhibition. The NADase activity of hSARM1 was measured at different NAD+ concentrations with and without the presence of 10 μM of the indicated inhibitors. The results showed that the examined molecules inhibited hSARM1 in a competitive manner.

FIG. 12D. Exemplary HPLC chromatograms. To determine the IC₅₀ values, the amount of ADPR production were measured by HPLC and calculated and plotted by GraphPad.

FIG. 13A-13B. Potency and mode of inhibition of hSARM1 by the chemical compounds

FIG. 13A. Chemical structure of an additional hSARM1 inhibitory compound TK174, as denoted by Formula III.

FIG. 13B. Determination of IC₅₀ values, as in FIG. 11.

FIG. 14A-14D: Structural, kinetic, and pharmacological characterization of the zebrafish (zf)SARM1

FIG. 14A. Selected representation of 2D class averages used for the 3D reconstruction of zfSARM1. The number of particles that were included in each class average are indicated.

FIG. 14B. Color coded (as in FIG. 11A) protein model (of hSARM1 PDB ID 7ANW) docked in a transparent 8.1 Å resolution cryo-EM density map refined with C1 symmetry (gray). ‘Top view’ refers to the aspect of the molecule showing the TIR (red) and SAM2 (cyan) domains closest to the viewer, while in the ‘bottom view’ the SAM1 domains are the closest.

FIG. 14C. Kinetic comparison between hSARM1 and zfSARM1 shows an overall similarity of the two, with close Km and Kcat values (upper panel). Inhibition by the substrate NAD+ is also observed in zfSARM1 with Ki of 1.3 mM, compared with a Ki of 0.7 mM for hSARM1 (lower panel). The kinetic parameters were determined from plots of reaction velocity of NAD+ consumption versus substrate (NAD+) concentration and then fitted to the Michaelis-Menten equation (Km and Vmax) or substrate inhibition equation (Ki) using non-linear curve fit in GraphPad Prism. Kcat was calculated by dividing the Vmax with protein molar concentration.

FIG. 14D. The effect of hSARM1 inhibitors on zfSARM1 NADase activity. An IC₅₀=227 μM for NAM demonstrates that zfSARM1 is also susceptible to inhibition by product, similar to hSARM1 (see FIG. 12B). The TK142 compound exhibits concentration-dependent inhibitory effects on zfSARM1. Remarkably, the TK138 compound has an opposite, activating effect, with ECS) of 2.7 μM. IC₅₀ and EC₅₀ values were determined according with ADPR production as measured in HPLC and calculated and plotted with GraphPad.

FIG. 15A-15C: hSARM1 inhibitory compounds protects axons against axotomy-induced degeneration

FIG. 15A. Mouse DRG explants cultures were grown in NGF containing media for 96 hours. Right before axotomy, the media was exchanged and supplemented with either 10-30 μM of the hSARM1 inhibitory compounds or just with 0.2% DMSO for control, and then the culture were left to grow for additional 16 hours. The white dashed lines indicate cut site (axotomy). To evaluate toxic side effects, we applied 20-3011M of the inhibitory compounds without performing axotomy. The axons were stained with anti-Tuj1 antibody.

FIG. 15B. Quantification of the protective (left) and toxic (right) effects induced by the hSARM1 inhibitory compounds. While TK138 provide the highest level of protection after axotomy, TK138 has also a significant toxic effect without axotomy. Data are presented as mean±SEM for 3 independent experiments. One-way ANOVA followed by Dunett post tests were used to determine statistical significance (*p <0.05, **p <0.01, ***p <0.0001).

All the analyses were performed in GraphPad Prism software.

FIG. 15C. Extended representation of FIG. 15A. Protective and toxic effects of hSARM1 inhibitors over axon degeneration in mouse DRG explants.

These and other features of the present disclosure will become apparent by the hand of the following description.

### DETAILED DESCRIPTION OF THE INVENTION

The disclosure presents structural data and complementary biochemical assays, to show that SARM1 is kept inactive through a ‘substrate inhibition’ mechanism, where high concentration of NAD+ stabilizes the tightly packed, inhibited conformation of the protein. In this way, SARM1 activation is aided by a decrease in the concentration of a cellular metabolite—NAD+.

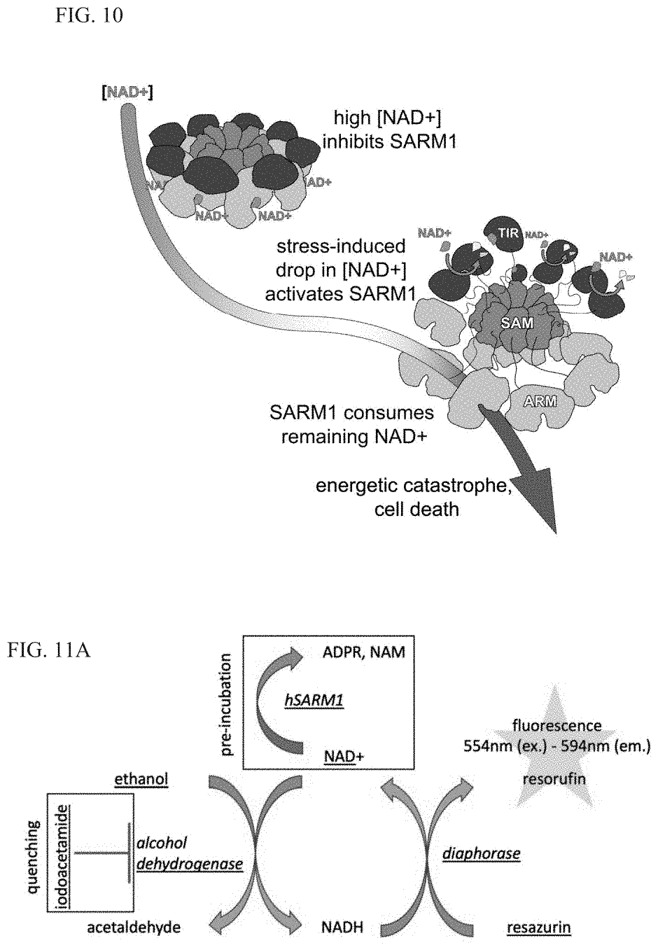

The octamer ring structure of a near-intact hSARM1 reveals an inhibited conformation, in which the catalytic TIR domains are kept apart from each other, unable to form close homodimers, which are required for their NADase activity. This inhibited conformation readily disassembles and gains most of its potential activity (Km of 28±4 μM, with Vmax of 9±0.3 μM/min and Kcat of 46.49 l/min) during protein purification, substantiating the hypothesis that a low-affinity cellular factor inhibits hSARM1, but is lost in purification. The inventors found that NAD+ induces a dramatic conformational shift in purified hSARM1, from ‘open’ to ‘compact’ conformations, through binding to a distal allosteric site from the TIR catalytic domain. Mutations in this allosteric site promoted hSARM1 activity in cultured cells, thereby demonstrating the key role of this site in inhibiting the NADase activity. The inventors also found that hSARM1 is inhibited for NADase activity in vitro by NAD+, demonstrating a ‘substrate inhibition mechanism’. Following these results, a model for hSARM1 inhibition in homeostasis and activation under stress (FIG. 10), is proposed herein. In this model, it is suggested that hSARM1 is kept inhibited by NAD+ through its allosteric site, located at the ARM domain horns' junction, that induces the compact inhibited conformation. This state persists as long as NAD+ remains at normal cellular levels. However, upon a drop in the cellular NAD+ concentration below a critical threshold, such as in response to stress, NAD+ dissociates from the allosteric inhibitory site. This triggers the disassembly of the compact conformation, and the dimerization of TIR domains, which enables TIR's NADase activity and NAD+ hydrolysis. The consequence is a rapid decrease in NAD+ cellular levels, leading to energetic catastrophe and cell death.

Understanding the mechanism of substrate inhibition of SARM1 enables to develop efficient assays for targeting SARM1 which represents an interesting molecular target for the development of safe and effective pharmacological therapy to protect axons in a variety of axonopathies.

Thus, according to first aspect, a screening method for identifying a modulator of sterile α and HEAT/armadillo motif-containing protein-1 (SARM1) NADase. In some embodiments, the modulator screened by the disclosed method an inhibitor of SARM1. In some specific embodiments, the method comprising the following steps:

First (a), incubating (i) at least one candidate compound; and (ii) at least one full length, or nearly full length catalytically active SARM1 or any fragment or variant thereof, or any mixture comprising (i) and (ii). In some embodiments, any fragment or variant of SARM1 that may be useful in the methods of the present disclosure comprises the TIR domain and the TIR docking site of the ARM-1 domain.

The next step (b), involves adding to the SARM1 and candidate, or mixture of (a), at least one SARM1 substrate or any analogues thereof, and incubating the mixture under suitable conditions.

In the next step (c), quantifying the amount of SARM1 substrate of any hydrolysis products thereof in the incubated mixture of (b); by a suitable means.

The final step (d) involves identifying the candidate compound as an inhibitor of SARM1 NADase. More specifically, a candidate compound is identified as an inhibitor of SARM1 NADase if step (c) of the method of the invention, results in at least one of the following outcomes in the presence of the candidate.

In some embodiments, a candidate compound is considered as an inhibitor if (i), the amount of the SARM1 substrate in the mixture of (c) is not reduced over time. In some embodiments, the SARM1 substrate is Nicotinamide adenine dinucleotide+(NAD+), or any analogues thereof.

It should be noted that since NAD+ is SARM substrate, reduction of NAD+ amount over the reaction time reflects SARM1 NADase activity. A candidate compound that attenuates or inhibits the reduction of SARM1 substrate (e.g., NAD+) amount, may be considered as inhibiting SARM1 NADase activity. In another alternative or additional embodiment (ii), a candidate compound may be considered as an inhibitor of SARM1 NADase activity, if the amount of SARM1 substrate, specifically, NAD+, in the mixture of (c), is greater than that of a control mixture that does not contain the candidate compound.

As indicated above, in some embodiments, the SARM1 substrate is Nicotinamide adenine dinucleotide+(NAD+), or any analogues thereof.

The present disclosure provides methods for identifying modulators of SARM1 NADase. The term “modulator” includes inhibitors and activators. Inhibitors are agents that inhibit, partially or totally block stimulation or activation, decrease, prevent, delay activation, inactivate, desensitize, or down regulate the SARM1 NADase activity in accordance with the invention, e.g., antagonists, or any indirect inhibitor/s. In accordance with the invention, activators are agents that induce, activate, stimulate, increase, facilitate, enhance activation, sensitize or up regulate the activation of SARM1 NADase activity, e.g., agonists, or any indirect activator. SARM1 NADase activity as used herein is the hydrolysis of its substrate NAD+, into ADP-ribose (ADPR), cyclic ADPR, and/or nicotinamide. Thus, in some embodiments, activation of SARM1 results in at least one of: hydrolysis of NAD+, production of hydrolysis products, specifically, ADPR and/or cADPR, and/or axonal and/or cellular degeneration and death. An inhibitor in accordance with the present disclosure, prevents and/or inhibits any of the specified activities of SARM1.

In some embodiments, the screening method of the invention is used for identifying inhibitors of SARM1 NADase. Such inhibitor as identified by the screening methods of the present disclosure may inhibits, reduces, attenuates, decreases the SARM1 NADase activity in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99%, 99.999%, 99.9999%, as compared to the SARM1 NADase activity in the absence of the inhibitor.

In some further embodiments. SARM1 inhibitor identified in the disclosed screening method inhibits SARM1 activity by at least 30%, specifically, leads to at least 30% reduction in SARM1 NADase activity. In yet some further embodiments, SARM1 inhibitor identified in the disclosed screening method inhibits SARM1 activity by at least 50%, specifically, leads to at least 50% reduction in SARM1 NADase activity. Still further, in some further embodiments, SARM1 inhibitor identified in the disclosed screening method inhibits SARM1 activity by at least about 30% to about 100%, about 30% to about 90%, about 30% to about 80%, about 30% to about 70%, about 30% to about 60%, about 30% to about 50%, about 30% to about 40%, reduction in SARM1 NADase activity. Still further, in some embodiments, the SARM1 inhibitor identified in the disclosed screening method inhibits SARM1 activity by at least about 40% to about 100%, about 40% to about 90%, about 40% to about 80%, about 40% to about 70%, about 40% to about 60%, about 40% to about 50%, reduction in SARM1 NADase activity. In yet some further embodiments, SARM1 inhibitor identified in the disclosed screening method inhibits SARM1 activity by at least about 50% to about 100%, about 50% to about 90%, about 50% to about 80%, about 50% to about 70%, about 50% to about 60%, reduction in SARM1 NADase activity. In yet some further embodiments, SARM1 inhibitor identified in the disclosed screening method inhibits SARM1 activity by at least about 60% to about 100%, about 60% to about 90%, about 60% to about 80%, about 60% to about 70%, reduction in SARM1 NADase activity. Still further, SARM1 inhibitor identified in the disclosed screening method inhibits SARM1 activity by at least about 70% to about 100%, about 70% to about 90%, about 70% to about 80%, reduction in SARM1 NADase activity. Still further, SARM1 inhibitor identified in the disclosed screening method inhibits SARM1 activity by at least about 80% to about 100%, about 80% to about 90%, reduction in SARM1 NADase activity. Still further, SARM1 inhibitor identified in the disclosed screening method inhibits SARM1 activity by at least about 90% to about 100%, reduction in SARM1 NADase activity.

It should be noted that the screening methods of the present disclosure may be a high throughput screening method. High-throughput screening (HTS), as used herein, is a method that may involve in some embodiments, robotics, data processing/control software, liquid handling devices, and sensitive detectors, allowing handling of an enormously large number of compounds. In yet some further embodiments, the HTS screening method may further comprise screening, identifying, evaluating and optimizing the selected candidate SARM1 modulator, specifically, inhibitor.

The present disclosure provides methods for screening for modulators, specifically, inhibitors of SARM1 NADase activity. SARM1 (sterile α and HEAT/armadillo motif-containing protein), also known as Myd88-5, is an evolutionarily ancient member of the Myd88 family of toll-like receptor adaptor proteins containing toll/interleukin-1 receptor (TIR) domains. SARM1 is normally kept in an inactive state by an interaction of the N-terminal armadillo domain with the C-terminal TIR domain. An intramolecular rearrangement in response to pathological triggers of axonal and/or cellular degeneration leads to dimerization of the TIR domains and to SARM1-dependent Wallerian degeneration of injured axons. The dimerization of the TIR domains is the critical and committed step in SARM1 activation.

SARM1 contains an N-terminal mitochondrial localization signal, an auto-inhibitory ARM-repeats region, two tandem SAM domains and a C-terminal TIR domain. The ARM domain interactions with the SAM and TIR domains prevent TIR NADase activity, and after injury-induced activation, the TIR domains become able to multimerize, leading to enzymatic degradation of NAD+. The SARM1 according to some embodiments. Is the human SARM1. In more specific embodiments, the SARM1 is the human SARM1 comprising the amino acid sequence as denoted b accession number Q6SZW1. Still further, in some embodiments, the SARM1 comprises the amino acid sequence as denoted by SEQ ID NO: 12.

As indicated above, for screening for SARM1 NADase inhibitors, a SARM1 molecule is used, specifically a nearly full length SARM1 polypeptide, as opposed to the use of only the catalytic domain TIR SARM1 or the SAM-TIR construct. More specifically, the term “full-length,” % Olen used to refer to SARM1, refers to a SARM1 polypeptide that comprises at least: (i) the N-terminal auto-inhibitory ARM domain or a functional fragment thereof. (ii) one or more SAM domains or a functional fragment thereof, and (iii) a TIR domain or a functional fragment thereof, of a human SARM1 polypeptide having NADase activity. In some embodiments, a nearly full-length SARM1 lacks several N-terminal amino acid residues, specifically, about 5 to 50 N-terminal amino acid residues. More specifically, a nearly full-length SARM1 lacks 5, 6, 7, 8, 9, 10, 11, 12, 13, 14, 15, 16, 17, 18, 19, 20, 21, 22, 23, 24, 25, 26, 27, 8, 29, 30, 31, 32, 33, 34, 35, 36, 37, 38, 39, 40, 41, 42, 43, 44, 45, 46, 47, 48, 49, 50, or more N-terminal amino acid residues. In some embodiments, a nearly full-length SARM1 lacks the 25 N-terminal amino acid residues of SARM1 (the wild type SARM1 is denoted by SEQ ID NO. 12). In some embodiments, provided are SARM1 polypeptides comprising at least a functional fragment of a SARM1 N-terminal auto-inhibitory ARM domain, at least a functional fragment of one or more SAM domains, and at least a functional fragment of a SARM1 TIR domain. In yet some further embodiments, a nearly full-length SARM may comprise residues 26+/−10 amino acid residues to 724+/−10 amino acid residues. In some embodiments, a nearly full-length SARM1 comprise the amino acid sequence of residues 26 to 723, 26 to 722, 26 to 721, 26 to 720, 26 to 719, 26 to 718, 26 to 717, 26 to 716, 26 to 715, 26 to 725, 26 to 726, 26 to 727, 26 to 728, 26 to 729, 26 to 730, 26 to 731, 26 to 732, 26 to 733, 25 to 724, 24 to 724, 23 to 724, 22 to 724, 21 to 724, 20 to 724, 19 to 724, 18 to 724, 17 to 724, 16 to 724, 27 to 724, 28 to 724, 29 to 724, 30 to 724, 31 to 724, 32 to 724, 33 to 724, 34 to 724, 35 to 724, 36 to 724, of the wild type SARM1 as denoted by SEQ ID NO. 12.

In yet some more specific embodiments, a nearly full-length SARM1 comprise the amino acid sequence of residues 26 to 724 of SARM1.

In yet some further embodiments, a full length, catalytically active SARM1 comprises an amino acid sequence of residues 1 to 724 of SARM1 as denoted by SEQ ID NO: 12, or any homologs, variants and derivatives thereof.

In some embodiments, the full length active SARM1 is encoded by a nucleic acid molecule comprising a nucleic acid sequence as denoted by the cDNA sequence of ENA|CAB90355|CAB90355.1 Homo sapiens (human) KIAA0524SARM. In yet some further specific embodiments, the full length active SARM1 is encoded by a nucleic acid molecule comprising the nucleic acid sequence as denoted by SEQ ID NO: 6, and any fragments, derivative, variants and homologs thereof.

In yet some further embodiments, the nearly full length SARM1 may comprise residues 26 to 724 of SARM, as denoted by SEQ ID NO. 13, or any fragments, derivative, variants and homologs thereof. Still further, in some embodiments, the nearly full length SARM1 used in the present invention may comprise residues 26 to 724 of SARM and any additional N-terminal sequence (for example, His-tag and the like), as denoted by SEQ ID NO. 5, or any fragments, derivative, variants and homologs thereof.

In some particular embodiments, the full length catalytically active SARM1 is a purified polypeptide.

As used herein, fragment or variant of SARM1, in accordance with the present disclosure, include but are not limited to TIR and ARM domains (residue 60 to 701 of the human wild type SARM1 presented by the amino acid sequence of SEQ ID NO. 12, specifically, as denoted by SEQ ID NO: 14), specifically, ARM-1 (residues 60-303, of the human wild type SARM1 presented by the amino acid sequence of SEQ ID NO. 12, specifically, as denoted by SEQ ID NO: 15) that comprises the TIR docking site, specifically, the ‘primary’ that engages the TIR helix α A and the EE loop with the ARM1 α 10, α 10-11 loop and a 13, and the ‘secondary’ TIR docking site is smaller and involves the TIR BB loop and helix α 7 of the counter-clockwise ARM1 domain. In yet some further embodiments, the SARM1 variant comprises at least one of residues L152, R157 and R322, intact. To evaluate the effect of a candidate compound on SARM NADase activity, the screening method of the present disclosure involves in some embodiments thereof, the use of a SARM substrate, and determination of the effect of the candidate compound on the amount of the substrate NAD+, as reflecting SARM NADase activity.

In some embodiments, the SARM1 substrate is Nicotinamide adenine dinucleotide+(NAD+), or any analogues thereof.

Nicotinamide adenine dinucleotide (NAD), also named Diphosphopyridine nucleotide (DPN+), Coenzyme I, (Cas number: 53-84-9) is a cofactor central to metabolism. Found in all living cells, NAD is called a dinucleotide because it consists of two nucleotides joined through their phosphate groups. One nucleotide contains an adenine nucleobase and the other nicotinamide. NAD exists in two forms: an oxidized and reduced form, abbreviated as NAD+ and NADH respectively.

Still further, some embodiments of the present disclosure encompass the use of analogues of NAD+ that may be also suitable for performing the screening method of the invention. Non-limiting examples for analogs of NAD+ applicable in the present disclosure, include oxidized forms of: nicotinamide 1, N6-ethenoadenine dinucleotide (eNAD), nicotinamide guanine dinucleotide, nicotinamide hypoxanthine dinucleotide, and nicotinamide hypoxanthine dinucleotide.

An initial step of the screening method of the present disclosure involves incubation of the candidate compound with the full length, or near full length SARM1, under suitable conditions. In some embodiments, such condition may include the incubation of both in a suitable temperature, specifically, any appropriate temperature ranging between 10° C. or lower to 40° C., or higher, specifically, about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. and 40° C. In some embodiments, such temperature may be 25° C. Still further, the candidate compound and near full length SARM1 may be incubated in a mixture comprising the candidate compound and SARM1, for an appropriate period ranging from 1 min to 30 minutes, specifically, 1, 2, 3, 4, 5, 6, 7, 8, 9, 10, 11, 12, 13, 14, 15, 16, 17, 18, 19, 20, 21, 22, 23, 24, 25, 26, 27, 8, 29, 30 minutes or more. In some specific embodiments, the candidate compound and near full length SARM1 may be incubated for 10 minutes. In yet some further embodiments, the candidate compound and near full length SARM1 may be incubated for 20 minutes. Still further, suitable conditions may according to some embodiments involve an appropriate pH conditions, specifically, a pH ranging between about pH 5 to about pH 9.5, specifically, pH 7.5. In some specific and non-limiting embodiments, the candidate compound and near full length SARM1 may be incubated at 25° C. for 10 to 20 minutes, in a buffer comprising 25 mM HEPES pH 7.5, 150 mM NaCl.

In yet some further embodiments, the steps of the methods of the invention that relate to incubation of the candidate compound and near full length SARM1 mixture with a SARM1 substrate, specifically, the NAD+ substrate, are performed under conditions suitable for performing NAD+ hydrolysis reaction. In some embodiments, the reaction may be performed in any appropriate temperature, ranging between 10° C. or lower to 40° C., or higher, specifically, about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C. and 40° C. In some embodiments, the reaction may be performed at Room temperature, specifically, a temperature ranging between about 18° C. to about 24° C., or specifically, between about 20° C. to about −22° C.

In yet some further embodiments, an appropriate temperature for such NAD+ hydrolysis reaction may be 37° C. Still further, suitable conditions may according to some embodiments involve an appropriate pH conditions, specifically, a pH ranging between about pH 5 to about pH 9.5, specifically, pH 7.5. In some specific and non-limiting embodiments, for NAD+ hydrolysis reaction, the reaction mixture may be incubated at 37° C. for about 70 minutes, in a buffer comprising 25 mM HEPES pH 7.5, 150 mM NaCl.

In some embodiments, the reaction is conducted during a period ranging from 1 min to 3 hours, specifically about 2-10 min, or about 5-10 min, or about 5-15 min, or about 5-20 min, about 5-25 min, about 5-30 min, or about 2-15 min, or about 2-20 min, or about 2-25 min, or about 2-30 min, or about 30 min-1 hour, or about 45 min to 1 hour, or about 1 hour to 1.5 hour, or about 1.5 hour to 2 hours, or about 2 hours to 3 hours, or about 2.5 hours to 3 hours or more. In some specific embodiments the reaction may be conducted for about 70 minutes.

As indicated above, following incubation of SARM1 with the candidate compound under conditions that allow contact between SARM1 and the candidate compound. NAD+ is added to the mixture under conditions suitable for NADase reaction, and the amount of NAD+ is determined.

In some embodiments, the amount of NAD+ may be quantified by at least one of, at least one fluorescence assay, at least one HPLC assay and at least one chemiluminescent assay.

In yet some further embodiments, the fluorescence assay used by the screening methods of the present disclosure may be at least one of a Resasurin NAD+ assay and eNAD-based NADase assay.

In some specific embodiments, the NAD+ amount may be determined by a Resasurin NAD+ assay. Resasurin NAD+ assay relates to a fluorescent assay enabling to measure NAD+ levels. Resazurin (7-Hydroxy-3H-phenoxazin-3-one 10-oxide) is a phenoxazine dye that is weakly fluorescent, nontoxic, cell-permeable, and redox-sensitive. Resazurin can be irreversibly reduced in the presence of NAD+ to the pink-colored and highly fluorescent resorufin (7-Hydroxy-3H-phenoxazin-3-one). At circum-neutral pH, resorufin can be detected by visual observation of its pink color or by fluorimetry, with an excitation maximum at 530-570 nm and an emission maximum at 580-590 nm. In some specific embodiments, the fluorescence is measured at 554 nm Excitation and 593 nm emission wave-length.

In some specific embodiments, the NAD+ amount may be determined by an eNAD-based NADase assay. As used herein, eNAD-based NADase assay relates to a fluorescent assay for measuring the NAD+ levels, using the NAD+ analog, etheno-NAD (eNAD—Nicotinamide 1,N6-ethenoadenine dinucleotide, for example the commercially available form SIGMA-N2630) that fluoresces upon hydrolysis with an excitation at λ=330 nm and an emission at λ=405 nm. In some further embodiments, the fluorescence is measured at 330 nm Excitation and 405 nm emission wave-length.

In yet some further embodiments, the NAD+ amount is determined by the methods of the invention using a reverse-phase HPLC.

High-performance liquid chromatography (HPLC), formerly referred to as high-pressure liquid chromatography, is a technique in analytical chemistry used to separate, identify, and quantify each component in a mixture. It relies on pumps to pass a pressurized liquid solvent containing the sample mixture through a column filled with a solid adsorbent material. Each component in the sample interacts slightly differently with the adsorbent material, causing different flow rates for the different components and leading to the separation of the components as they flow out of the column. HPLC assay may therefore be used in order to evaluate NAD+ hydrolysis or consumption by examining the elution of NAD+ and the elution of the NAD+ hydrolysis product i.e. ADP Ribose (ADPR) and/or Cyclic ADP Ribose (cADPR) and/or NAM (nicotinamide).

In some embodiments, for evaluating the amount of NAD+, and thereby to assess SARM1 NADase activity, the methods of the present disclosure may measure NAD+ hydrolysis products. In certain embodiments, the NAD+ hydrolysis product may be ADPR and/or cADPR.

In some further embodiments, NADase activity may be activated by addition of nicotinamide mononucleotide (NMN) or analogue thereof, as exemplified in Example 3. In some embodiments, the concentration of the NMN supplement may be between 0.1 mM to 10 mM, specifically, 0.1, 0.2, 0.3, 0.4, 0.5, 0.6, 0.7, 0.8, 0.9, 1, 2, 3, 4, 5, 6, 7, 8, 9 or 10 mM, for example at a concentration of 0.2 mM or 1 mM.

Still further, as indicated above, for evaluating the candidate compound, the method of the invention may further determine the effect of the compound on SARM1 conformation, as reflecting its activity. As demonstrated in the examples, a close-two-ring conformation reflects inhibitory conformation of SARM1. Thus, candidate compounds that transform, either directly or indirectly, SARM1 conformation from the open one-ring conformation to the close two-ring inhibitory conformation, may be considered as SARM1 NADase inhibitor.

A “close two-ring inhibitory conformation”, as used herein is a compact conformation where the catalytic TIR domains are docked on ARM domains apart from each other, unable to form close dimers required for NAD+ catalysis.

An “open one-ring conformation”, as used herein, involves disintegration of the ARM-TIR outer ring assembly, while the constitutively-assembled SAM inner ring is present.

In some embodiment, the SARM1 conformation may be determined by cryo-electron microscope (cryo-EM). As used herein, Cryogenic electron microscopy (cryo-EM) is an electron microscopy (EM) technique applied on samples cooled to cryogenic temperatures and embedded in an environment of vitreous water. An aqueous sample solution is applied to a grid-mesh and plunge-frozen in liquid ethane or a mixture of liquid ethane and propane. Recent advances in detector technology and software algorithms have allowed for the determination of biomolecular structures at near-atomic resolution. This has attracted wide attention to the approach as an alternative to X-ray crystallography or NMR spectroscopy for macromolecular structure determination without the need for crystallization.

In some embodiments, a candidate compound that has been identified by the screening method disclosed herein, as a potential inhibitor of SARM1, may be further evaluate by its effect on cell viability. Thus, in some embodiments, the disclosed screening method may further comprise the step of evaluating the effect of the candidate compound on cell viability. It should be understood that in some embodiments, an effective SARM1 NADase inhibitor is expected to increase cell viability. More specifically, in some embodiments, Cell viability is a measure of the proportion of live, healthy cells within a population.

Thus, in some embodiments, a candidate compound determined by the screening methods of the invention as a potential SARM1 NADase inhibitor, may be further evaluated by a cell viability assay.

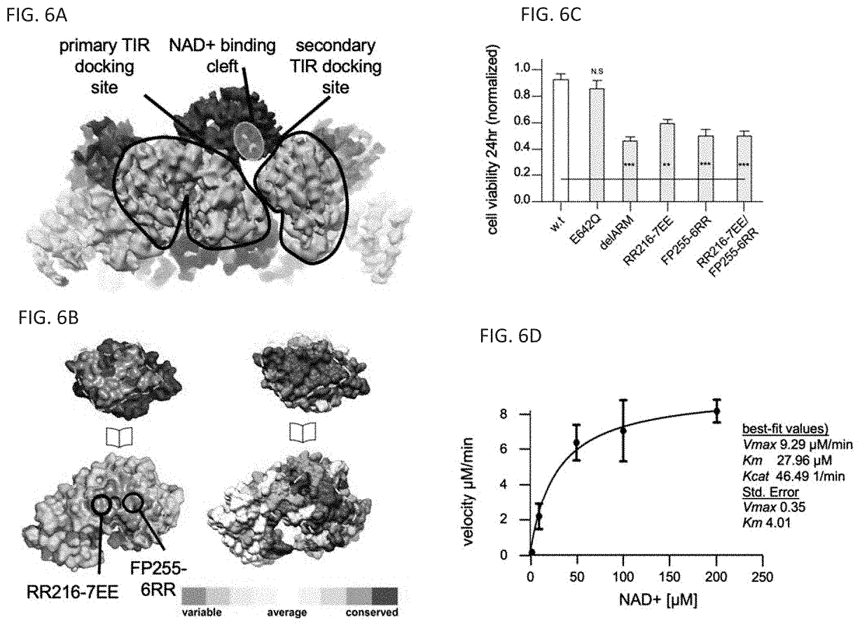

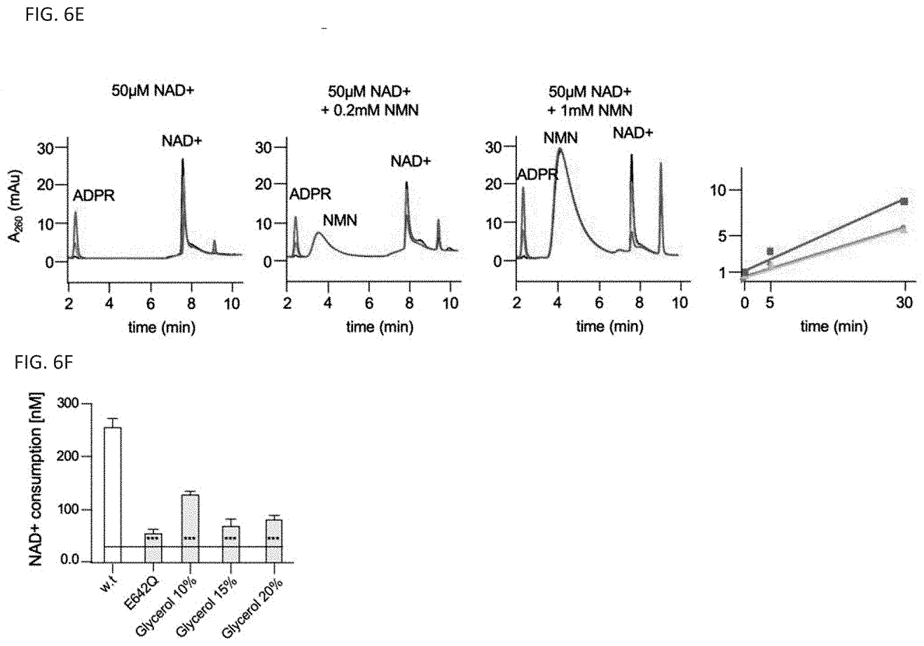

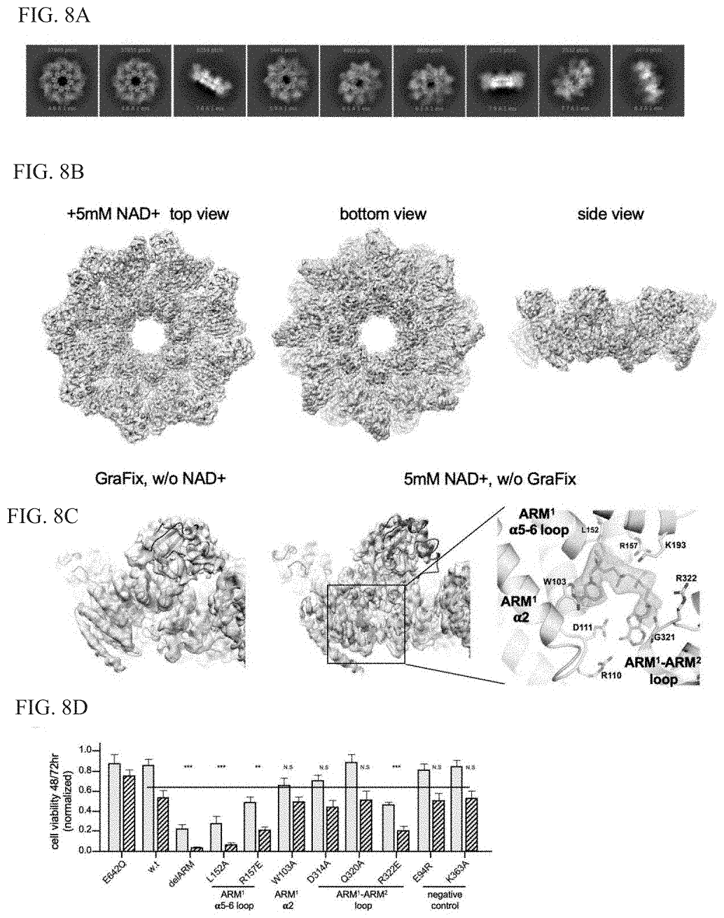

In some embodiments, a cell viability assay may be further conducted as shown in Example 2 (FIG. 6C) and Example 4 (FIG. 8D). As used herein, a cell viability assay refers to is an assay for determining the ability of cells to maintain or recover a state of survival. In some embodiments, assays for cell viability applicable in the present disclosure include but are not limited to fluorescent resazurin assay, MTT (tetrazolium dye MTT 3-(4,5-dimethylthiazol-2-yl)-2,5-diphenyltetrazolium bromide) assay, XTT (2,3-bis-(2-methoxy-4-nitro-5-sulfophenyl)-2H-tetrazolium-5-carboxanilide) assay, MTS (3-(4,5-dimethylthiazol-2-yl)-5-(3-carboxymethoxyphenyl)-2-(4-sulfophenyl)-2H-tetrazolium) assay, WST (water-soluble tetrazolium salts) assay, ATP uptake assay and glucose uptake assay.

Still further, in some embodiments the effect of the candidate compound that exhibits SARM1 NADase inhibitory activity may be evaluated using any neuronal cell culture, specifically, the protective effect on the viability, and/or function of the cells. In yet some further embodiments, the candidate SARM1 NADase inhibitor identified by the disclosed screening method may be further evaluated using any suitable animal model. In some embodiments, as also exemplified by Example 8, the protective effect may be evaluated using the axon degradation assay after axotomy. More specifically, an in vitro assay to assess the axonal fragmentation induced by mechanical injury to axons in cultured mouse embryonic dorsal root ganglion (DRG) neurons. DRG neurons are pseudounipolar and therefore suitable for an assay of axonal degeneration after injury. In addition, the time course of the axonal fragmentation is stereotyped, enabling the identification of reagents that either expedite or impede the degeneration process. With an image-based quantification method, the in vitro degeneration assay is utilized for evaluating the effect of the selected identified candidate SARM1 inhibitors on delaying axon degeneration.

The term “polypeptide” as used herein refers to amino acid residues, connected by peptide bonds. A polypeptide sequence is generally reported from the N-terminal end containing free amino group to the C-terminal end containing free carboxyl group and may include any polymeric chain of amino acids. In some embodiments, a polypeptide has an amino acid sequence that occurs in nature. In some embodiments, a polypeptide has an amino acid sequence that does not occur in nature. In some embodiments, a polypeptide has an amino acid sequence that contains portions that occur in nature separately from one another (i.e., from two or more different organisms, for example, human and non-human portions). In some embodiments, a polypeptide has an amino acid sequence that is engineered in that it is designed and/or produced through action of the hand of man. More specifically, “Amino acid sequence” or “peptide sequence” is the order in which amino acid residues connected by peptide bonds, lie in the chain in peptides and proteins. The sequence is generally reported from the N-terminal end containing free amino group to the C-terminal end containing amide. Amino acid sequence is often called peptide, protein sequence if it represents the primary structure of a protein, however one must discern between the terms “Amino acid sequence” or “peptide sequence” and “protein”, since a protein is defined as an amino acid sequence folded into a specific three-dimensional configuration and that had typically undergone post-translational modifications, such as phosphorylation, acetylation, glycosylation, manosylation, amidation, carboxylation, sulfhydryl bond formation, cleavage and the like.

Still further, the SARM1 full-length, or nearly full-length polypeptide that may be used by the present disclosure may comprise in some embodiments the amino acid sequence of SEC ID NO. 12 (full length), the sequence of residues 26 to 724, as denoted by SEQ ID NO. 13, and/or the nearly full length SARM1 as denoted by SEQ ID NO. 5, or of any variants, fragments and homologs thereof.

By “fragments or peptides” it is meant a fraction of the protein of the invention. A “fragment” of a molecule, such as any of the amino acid sequences of the present invention, is meant to refer to any amino acid subset. This may also include “variants” or “derivatives” thereof. A “peptide” is meant to refer to a particular amino acid subset having a functional, structural activity or function displayed by the protein disclosed by the invention.

It should be appreciated that the invention encompasses any variant or derivative of the SARM1 protein of the invention and any polypeptides that are substantially identical or homologue. The term “derivative” is used to define amino acid sequences (polypeptide), with any insertions, deletions, substitutions and modifications to the amino acid sequences (polypeptide) that either do not alter the activity of the original polypeptides or alter it purposefully. In this connection, a derivative or fragment of the variant of the invention may be any derivative or fragment of the variant and/or mutated molecule, specifically as denoted by SEQ ID NO. 12, SEQ ID NO. 13, and/or SEQ ID NO. 5, that do not reduce or alter the activity of the variant of the invention.

By the term “derivative” it is also referred to homologues, variants and analogues thereof. Proteins orthologs or homologues having a sequence homology or identity to the proteins of interest in accordance with the invention, specifically that may share at least 50%, at least 60% and specifically 65%, 70%, 75%, 80%, 85%, 90%, 91%, 92%, 93%, 94%, 95%, 96%, 97%, 98%, 99%, 100%, specifically as compared to the entire sequence of the proteins of interest in accordance with the invention, for example, as denoted by SEQ ID NO. 12, SEQ ID NO. 13, and/or SEQ ID NO. 5. Specifically, homologs that comprise or consists of an amino acid sequence that is identical in at least 50%, at least 60% and specifically 65%, 70%, 75%, 80%, 85%, 90%, 91%, 92%, 93%, 94%, 95%, 96%, 97%, 98%, 99%, or higher to SEQ ID NO. 12 SEQ ID NO. 13, and/or SEQ ID NO. 5.

In some embodiments, derivatives refer to polypeptides, which differ from the polypeptides specifically defined in the present invention by insertions, deletions or substitutions of amino acid residues. It should be appreciated that by the terms “insertion/s”, “deletion/s” or “substitution/s”, as well as “substituted, “deleted”, “inserted”, as used herein it is meant any addition, deletion or replacement, respectively, of amino acid residues to the polypeptides disclosed by the invention, of between 1 to 50 amino acid residues, between 20 to 1 amino acid residues, and specifically, between 1 to 10 amino acid residues. More particularly, insertion/s, deletions or substitutions may be of any one of 1, 2, 3, 4, 5, 6, 7, 8, 9, or 10 amino acids. It should be noted that the insertion/s, deletion/s or substitution/s encompassed by the invention may occur in any position of the modified peptide, as well as in any of the N′ or C′ termini thereof.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

For example, substitutions may be made wherein an aliphatic amino acid (G, A, I, L, or V) is substituted with another member of the group, or substitution such as the substitution of one polar residue for another, such as arginine for lysine, glutamic for aspartic acid, or glutamine for asparagine. Each of the following eight groups contains other exemplary amino acids that are conservative substitutions for one another:

### 1) Alanine (A), Glycine (G);

2) Aspartic acid (D). Glutamic acid (E):

### 3) Asparagine (N), Glutamine (Q):

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M).

More specifically, amino acid “substitutions” are the result of replacing one amino acid with another amino acid having similar structural and/or chemical properties, i.e., conservative amino acid replacements. Amino acid substitutions may be made on the basis of similarity in polarity, charge, solubility, hydrophobicity, hydrophilicity, and/or the amphipathic nature of the residues involved. For example, nonpolar “hydrophobic” amino acids are selected from the group consisting of Valine (V), Isoleucine (I), Leucine (L), Methionine (M), Phenylalanine (F), Tryptophan (W), Cysteine (C), Alanine (A), Tyrosine (Y), Histidine (H), Threonine (T), Serine (S), Proline (P), Glycine (G), Arginine (R) and Lysine (K); “polar” amino acids are selected from the group consisting of Arginine (R), Lysine (K), Aspartic acid (D), Glutamic acid (E), Asparagine (N), Glutamine (Q); “positively charged” amino acids are selected form the group consisting of Arginine (R), Lysine (K) and Histidine (H) and wherein “acidic” amino acids are selected from the group consisting of Aspartic acid (D), Asparagine (N), Glutamic acid (E) and Glutamine (Q).

Variants of the polypeptides of the invention may have at least 80% sequence similarity or identity, often at least 85% sequence similarity or identity. 90% sequence similarity or identity, or at least 95%, 96%, 97%, 98%, or 99% sequence similarity or identity at the amino acid level, with the protein of interest, such as the various polypeptides of the invention.

Still further, in some embodiments, a variant of nearly full length SARM1 may comprise any sequence derived from residues 26 to 724 of the human ARM1 as denoted by SEQ ID NO. 13, or any homologues, variants and derivatives thereof, provided that such sequence comprises at least one of, the TIR and ARM domains (residue 60 to 701 of SARM1 presented by the amino acid sequence of SEQ ID NO. 12), specifically, ARM-1 (residues 60-304) that comprises the TIR docking site, specifically, the ‘primary’ that engages the TIR helix α A and the EE loop with the ARM1 α 10, α 10-11 loop and α 13, and the ‘secondary’ TIR docking site is smaller and involves the TIR BB loop and helix α 7 of the counter-clockwise ARM1 domain. In yet some further embodiments, said SARM1 variant comprises at least one of residues L152, R157 and R322, intact. In some embodiments, a nearly full length SARM1 may comprise the amino acid sequence as denoted by SEQ ID NO. 5, or any homologues, variants and derivatives thereof. Still further, in some embodiments, the SARM1 and/or NAD+ molecules used by the methods of the present disclosure may be tagged with a detectable moiety, or any moiety that allows separation of the labeled SARM and/or NAD+. In some further embodiments, the detectable moiety associated with the SARM1 and/or NAD+ molecules used by the method of the invention may refer to any chemical moiety that can be used to provide a detectable signal, and that can be attached to a nucleic acid or protein via a covalent bond or noncovalent interaction (e.g., through ionic or hydrogen bonding, or via immobilization, adsorption, or the like).

Labels generally provide signals detectable by fluorescence, chemiluminescence, radioactivity, colorimetry, mass spectrometry, X-ray diffraction or absorption, magnetism, enzymatic activity, or the like. Examples of labels include haptens, enzymes, enzyme substrates, coenzymes, enzyme inhibitors, fluorophores, quenchers, chromophores, magnetic particles or beads, redox sensitive moieties (e.g., electrochemically active moieties), luminescent markers, radioisotopes (including radionucleotides), and members of binding pairs.

More specific examples include fluorescein, phycobiliprotein, tetraethyl rhodamine, and beta-galactosidase. Binding pairs may include biotin/Strepavidin, biotin/avidin, biotin/neutravidin, biotin/captavidin, GST/glutathione, maltose binding protein/maltose, calmodulin binding protein/calmodulin, enzyme-enzyme substrate, receptor-ligand binding pairs, and analogs and mutants of the binding pairs.

In some further embodiments, the SARM1 and/or NAD+ molecules may be labeled or tagged. In some embodiments, the term “labeled” or “tagged” may refer to direct labeling of the protein via, e.g., coupling (i.e., physically linking) or incorporating of a detectable substance, or a “separation substance”, to the protein. Useful labels in the present invention may include but are not limited to include isotopes (e.g. 13C, 15N), or any other radiolabels (e.g., 3H, 125I, 35S, 14C, or 32P), magnetic beads (e.g. DYNABEADS), fluorescent dyes (e.g., fluorescein isothiocyanate, Texas red, rhodamine, green fluorescent protein, and the like), enzymes (e.g., horseradish peroxidase, alkaline phosphatase and others commonly used in an ELISA and competitive ELISA, histochemistry and other similar methods known in the art) and colorimetric labels such as colloidal gold or colored glass or plastic (e.g. polystyrene, polypropylene, latex, etc.) beads.

In some embodiments, the SARM1 and/or NAD+ molecules may be tagged. Different tags may be also used, for example, His, myc, HA, GFP, ABP, GST, biotin, FLAG and the like.

In some embodiments, an inhibitor of SARM1 NADase activity may be any compound that inhibits, reduces, attenuates, decreases the SARM1 NADase activity in About 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999%, as compared to the SARM1 NADase activity in the absence of the inhibitor.

In some further embodiments, SARM1 inhibitor inhibits SARM1 activity by at least 30%, specifically, leads to at least 30% reduction in SARM1 NADase activity. In yet some further embodiments, SARM1 inhibitor disclosed herein inhibits SARM1 activity by at least 50%, specifically, leads to at least 50% reduction in SARM1 NADase activity. Still further, in some further embodiments, SARM1 inhibitor disclosed herein inhibits SARM1 activity by at least about 30% to about 100%, about 30% to about 90%, about 30% to about 80%, about 30% to about 70%, about 30% to about 60%, about 30% to about 50%, about 30% to about 40%, reduction in SARM1 NADase activity. Still further, in some embodiments, the SARM1 inhibitor disclosed herein inhibits SARM1 activity by at least about 40% to about 100%, about 40% to about 90%, about 40% to about 80%, about 40% to about 70%, about 40% to about 60%, about 40% to about 50%, reduction in SARM1 NADase activity. In yet some further embodiments, SARM1 disclosed herein inhibits SARM1 activity by at least about 50% to about 100%, about 50% to about 90%, about 50% to about 80%, about 50% to about 70%, about 50% to about 60%, reduction in SARM1 NADase activity. In yet some further embodiments, SARM1 inhibitor disclosed herein inhibits SARM1 activity by at least about 60% to about 100%, about 60% to about 90%, about 60% to about 80%, about 60% to about 70%, reduction in SARM1 NADase activity. Still further, SARM1 inhibitor disclosed herein inhibits SARM1 activity by at least about 70% to about 100%, about 70% to about 90%, about 70% to about 80%, reduction in SARM1 NADase activity. Still further, SARM1 inhibitor disclosed herein inhibits SARM1 activity by at least about 80% to about 100%, about 80% to about 90%, reduction in SARM1 NADase activity. Still further, SARM1 inhibitor disclosed herein inhibits SARM1 activity by at least about 90% to about 100%, reduction in SARM1 NADase activity.

It should be noted that the SARM NADase inhibitor identified by the screening method of the present disclosure may be any competitive, non-competitive and/or allosteric inhibitor.

More specifically, the use of the full length, or nearly full length SARM1, that include all active and regulatory domains of the molecule facilitates the identification of any inhibitor that interacts directly or indirectly with any residue or domain of SARM1, thereby affecting the SARM1 NADase activity. Such inhibitor may in some embodiments interact either directly or indirectly with the TIR SARM1 catalytic domain, or specifically in the substrate binding site, and may compete, inhibit, or block substrate recognition and/or substrate binding. In yet some further embodiments, the inhibitor may interact directly or indirectly with any domain of the SARM1 molecule and affect the catalytic NADase activity of SARM1. Still further, the use of a full length or nearly full length SARM1 may facilitate and/or allow the identification of inhibitors that bind other domains of SARM and induce an inhibitory conformation.

Thus, in some embodiments, the inhibitor identified by the screening method of the invention may be a competitive inhibitor. More specifically, in some embodiments, a competitive inhibitor competes with the substrate for the active site, specifically, the substrate binding site. In competitive inhibition, the maximum velocity of the reaction is unchanged, while the apparent affinity of the substrate to the binding site is decreased.

In yet some other embodiments, the inhibitor identified by the screening method of the invention may be a non-competitive inhibitor. More specifically, most common mechanism of non-competitive inhibition involves reversible binding of the inhibitor to an allosteric site, but it is possible for the inhibitor to operate via other means including direct binding to the active site. It differs from competitive inhibition in that the binding of the inhibitor does not prevent binding of substrate, and vice versa, but simply prevents product formation for a limited time. This type of inhibition reduces the maximum rate of a chemical reaction without changing the apparent binding affinity of the catalyst for the substrate. When a non-competitive inhibitor is added the Vmax is changed, while the Km (affinity) remains unchanged.

As indicated above, the use of a near full-length SARM1, or the full length SARM1 in the screening methods of the present disclosure also (but not only) allows the identification of inhibitors that stabilize various domain of the SARM1 in a close conformation, or a two-ring inhibitory conformation, as defined herein before.

Thus, in yet some further specific embodiments, the SARM1 NADase inhibitor identified by the screening method of the invention may stabilize a SARM1 two-ring compact inhibitory conformation by binding at least one of the ARM; TIR-ARM; ARM-ARM; and ARM-SAM inter- and intra-domains interfaces of SARM1.

In some specific embodiments, the ARM1 domain may comprise an amino acid sequence as denoted by SEQ ID NO: 7, and any fragments, derivative, variants and homologs thereof. In some embodiments, the ARM2 domain may comprise an amino acid sequence as denoted by SEQ ID NO: 8 and any fragments, derivative, variants and homologs thereof. In some embodiments, the SAM1 domain may comprise an amino acid sequence as denoted by SEQ ID NO: 9, and any fragments, derivative, variants and homologs thereof. In some embodiments, the SAM2 domain may comprise an amino acid sequence as denoted by SEQ ID NO: 10, and any fragments, derivative, variants and homologs thereof. In some embodiments, the TIR domain may comprise an amino acid sequence as denoted by SEQ ID NO: 11, and any fragments, derivative, variants and homologs thereof.

In some embodiments, the inhibitor identified by the screening methods of the present disclosure may be an allosteric inhibitor.

Allosteric regulation is the regulation of a protein/enzyme by binding an effector molecule at a site other than the catalytic site of the protein/enzyme. The binding site is termed the allosteric site or regulatory site. Allosteric sites allow effectors to bind to the protein, often resulting in a conformational change involving protein dynamics. Effectors that enhance the activity of the protein/enzyme are referred to as allosteric activators, whereas those that decrease the protein's activity are called allosteric inhibitors.

In some embodiments, the inhibitor identified by the method of the invention does not directly interfere or compete with substrate binding at the TIR domain.

In some embodiments, the candidate compound used in the screening methods of the present disclosure, may be at least one of a small molecule, a nucleic acid molecule, an amino acid based molecule, a lipid, a polysaccharides and any combinations thereof.

A “Compound”, or a “candidate compound”, is used herein to refer to any substance, agent (e.g., molecule), supramolecular complex, material, or combination or mixture thereof. A compound may be any agent that can be represented by a chemical formula, chemical structure, or sequence. Example of compounds applicable for the present disclosure, include, e.g., small molecules, nucleic acid molecules (e.g., RNAi agents, antisense oligonucleotide, gRNAs, aptamers), amino acid based molecules, for example, polypeptides, peptides, antibodies specific for at least on of the full length SARM1, the nearly full length SARM1, the ARM; TIR-ARM; ARM-ARM; and ARM-SAM inter- and intra-domains interfaces of SARM1, that inhibit or disturb the NADase activity thereof, lipids, polysaccharides, etc. It should be understood that any compound described in connection to the present aspect is also applicable in all aspects of the invention. It should be further understood that the invention encompasses the use of any of the described compounds or any combinations or mixtures thereof. In general, candidate compounds may be obtained using any suitable method known in the art. The ordinary skilled artisan will select an appropriate method based, e.g., on the nature of the compound. A compound may be at least partly purified. In some embodiments a compound may be provided as part of a composition, which may contain, e.g., a counter-ion, aqueous or non-aqueous diluent or carrier, buffer, preservative, or other ingredient, in addition to the compound, in various embodiments. In some embodiments a compound may be provided as a salt, ester, hydrate, or solvate. In some embodiments a compound is cell-permeable, e.g., within the range of typical compounds that are taken up by cells and acts intracellularly, e.g., within mammalian cells, to produce a biological effect. Certain compounds may exist in particular geometric or stereoisomeric forms. Such compounds, including cis- and trans-isomers, E- and Z-isomers, R- and S-enantiomers, diastereomers, (D)-isomers. (L)-isomers, (−)- and (+)-isomers, racemic mixtures thereof, and other mixtures thereof are encompassed by this disclosure in various embodiments unless otherwise indicated. Certain compounds may exist in a variety or protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

Still further, in some embodiments, a candidate compound used in the screening methods of the present disclosure may be a small molecule compound. A “small molecule” as used herein, is an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da. 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups. Examples for small molecule compounds identified by the screening methods of the present disclosure are disclosed in Examples 6-8.

In some embodiments, a candidate compound screened by the screening methods of the invention may comprise at least one aptamer that is specific for at least one of the ARM; TIR-ARM; ARM-ARM; and ARM-SAM inter- and intra-domains interfaces of SARM1.

As used herein the term “aptamer” or “specific aptamers” denotes single-stranded nucleic acid (DNA or RNA) molecules which specifically recognizes and binds to a target molecule. The aptamers according to the invention may fold into a defined tertiary structure and can bind a specific target molecule with high specificities and affinities. Aptamers may be usually obtained by selection from a large random sequence library, using methods well known in the art, such as SELEX and/or Molinex. In various embodiments, aptamers may include single-stranded, partially single-stranded, partially double-stranded or double-stranded nucleic acid sequences; sequences comprising nucleotides, ribonucleotides, deoxyribonucleotides, nucleotide analogs, modified nucleotides and nucleotides comprising backbone modifications, branch points and non-nucleotide residues, groups or bridges; synthetic RNA, DNA and chimeric nucleotides, hybrids, duplexes, heteroduplexes; and any ribonucleotide, deoxyribonucleotide or chimeric counterpart thereof and/or corresponding complementary sequence. In certain specific embodiments, aptamers used by the invention are composed of deoxyribonucleotides.

In some further embodiments, the inhibitor identified by the screening method of the present disclosure may be adapted and/or suitable for use in the treatment of conditions or disorders associated directly or indirectly with axonal and/or cellular degradation. Axonal and/or cellular degradation is a hallmark of peripheral neuropathy, brain injury, and neurodegenerative disease. Neurodegeneration and neurodegenerative disorders include progressive structural and/or functional loss of nerve cells or neurons in the peripheral nervous system (PNS) and/or central nervous system (CNS). Axon degeneration has been proposed to be mediated by an active auto-destruction program, akin to apoptotic cell death.

More specifically, in many neurodegenerative diseases, prominent axonal pathology often precedes cell body loss in the form of “dying back,” in which axons from the synaptic regions gradually degenerate toward the cell body. Despite differences in the rate of degeneration, this process mirrors many morphological features of transected nerves, including axonal swelling, microtubule disassembly, and eventual fragmentation of the axonal cytoskeleton. The degeneration starts at a single point and finally involves the entire axon distal of the injured part. This process is referred to as anterograde (Wallerian) degeneration. The one involving the part of the axon proximal to the injury is called retrograde degeneration. The structural changes follow a certain time course and develop gradually. Initially, axon swelling and disintegration of neurofilaments and microtubules occurs. The axolemma becomes discontinuous, and retraction and disintegration of myelin ensues. These changes result in the progressive disintegration of the axon and myelin. Fragments of the axon often contain large amounts of myelin and are called myelin ovoid. Schwarm cells proliferate and ingest the degenerating axon.

In some embodiments, axonal and/or cellular degeneration is associated with at least one of a neurodegenerative or neurological disorder, axonal damage or injury, axonopathy, a demyelinating disease, a central pontine myelinolysis, a metabolic disease, a mitochondria) disease, metabolic axonal degeneration, axonal damage resulting from a leukoencephalopathy or a leukodystrophy.

Neurodegeneration is the umbrella term for the progressive loss of structure or function of neurons, including synaptic dysfunction and death of neurons. Many neurodegenerative diseases including Alzheimer's and Parkinson's are associated with neurodegenerative processes. Other examples of neurodegeneration that may be also applicable herein may include Friedreich's ataxia, Lewy body disease, spinal muscular atrophy, multiple sclerosis, frontotemporal dementia, corticobasal degeneration, progressive supranuclear palsy, multiple system atrophy, hereditary spastic paraparesis, amyloidosis, Amyotrophic lateral sclerosis (ALS), and Charcot Marie Tooth. It should not be overlooked that normal aging processes include progressive neurodegeneration, specifically, age-related cognitive decline (ACD) and mild cognitive impairment (MCI) are also applicable in the present invention. In yet some further embodiments, the methods, kits, compositions and cells of the invention may be applicable for Duchenne muscular dystrophy (DMD), or any conditions associated therewith. In yet some further embodiments, the methods, kits, compositions and cells of the invention may be applicable for Becker muscular dystrophy. In yet some further embodiments, the methods, kits, compositions and cells of the invention may be applicable for Tuberous sclerosis complex (TSC).

In some specific embodiments, the inhibitor identified by the screening methods of the invention may be used, may be adapted for use, may be suitable for use, in treating disorders such as any one of amyotrophic lateral sclerosis (ALS), Multiple Sclerosis (MS), Parkinson's disease (PD), Peripheral Nervous System (PNS) disorders, Alzheimer's disease (AD), ocular disorder and traumatic brain injury.

In more specific embodiments, the inhibitors identified by the method of the present disclosure may be applicable for ALS. Amyotrophic lateral sclerosis (ALS), also known as Lou Gehrig's disease and as motor neurone disease (MND) is a neurodegenerative neuromuscular disease that results in the loss of motor neurons that control voluntary muscles. ALS is the most common type of motor neuron disease. Early symptoms of ALS include stiff muscles, muscle twitches, and a gradual increasing weakness due to muscle wasting resulting from lack of muscle use. About half of the people affected develop at least mild difficulties with thinking and behavior and most people experience pain. Most eventually lose the ability to walk, use their hands, speak, swallow, and breathe. The cause is not known in 90% to 95% of cases but is believed to involve both genetic and environmental factors. The remaining 5-10% of cases are inherited from a person's parents. More than 20 genes have been associated with familial ALS, of which four account for the majority of familial cases: C9orf72 (40%), SOD1 (20%), FUS (1-5%), and TARDBP (1-5%). The underlying mechanism involves damage to both upper and lower motor neurons.

In more specific embodiments, the inhibitors identified by the method of the present disclosure may be applicable for MS. The term “Multiple Sclerosis” (MS) as herein defined is a chronic inflammatory neurodegenerative disease of the central nervous system that destroys myelin, oligodendrocytes and axons. MS is the most common neurological disease among young adults, typically appearing between the ages of 20 and 40. The symptoms of MS vary, from the appearance of visual disturbance such as visual loss in one eye, double vision to muscle weakness fatigue, pain, numbness, stiffness and unsteadiness, loss of coordination and other symptoms such as tremors, dizziness, slurred speech, trouble swallowing, and emotional disturbances. As the disease progresses patients may lose their ambulation capabilities, may encounter cognitive decline, loss of self-managing of everyday activities and may become severely disabled and dependent.

MS symptoms develop because immune system elements attack the brain's cells, specifically, glia and/or neurons, and damage the protective myelin sheath of axons. The areas in which these attacks occur are called lesions that disrupt the transmission of messages through the brain. Multiple sclerosis is classified into four types, characterized by disease progression: (1) Relapsing-remitting MS (RRMS), which is characterized by relapse (attacks of symptom flare-ups) followed by remission (periods of stabilization and possible recovery; while in some remissions there is full recovery, in other remissions there is partial or no recovery). Symptoms of RRMS may vary from mild to severe, and relapses may last for days or months. More than 80 percent of people who have MS begin with relapsing-remitting cycles; (2) Secondary-progressive MS (SPMS) develops in people who have relapsing-remitting MS. In SPMS, relapses may occur, but there is no remission (stabilization) for a meaningful period of time and the disability progressively worsens; (3) Primary-progressive MS (PPMS), which progresses slowly and steadily from its onset and accounts for less than 20 percent of MS cases. There are no periods of remission, and symptoms generally do not decrease in intensity; and (4) Progressive-relapsing MS (PRMS). In this type of MS, people experience both steadily worsening symptoms and attacks during periods of remission. It should be understood that the SARM NADase inhibitor of the present disclosure an any methods thereof, may be applicable for any type, stage or condition of the MS patient. Treatment using the inhibitors and methods of the invention may result in some embodiments in alleviation of any symptoms, and/or in prolonging the remission period between attacks.

In more specific embodiments, the inhibitors identified by the method of the present disclosure may be applicable for PD. “Parkinson's disease (PD)” as used herein, is a neurodegenerative disease resulting from degeneration of midbrain dopamine neurons and accumulation of alpha-synuclein containing Lewy bodies in surviving neurons. The diagnosis of PD is based on the presence of cardinal motor features in the absence of other aetiological conditions. These motor features include the classical triad of bradykinesia, a resting pill-rolling tremor, and rigidity typically in association with hypomimia, hypophonia, micrographia and postural instability. Non-motor features of PD may even precede its diagnosis, constituting prodromal or premotor PD. These premotor features include problems with olfaction, constipation, mood and sleep, and following the clinical diagnosis of PD, they can become more prominent. Cognitive problems and dementia also commonly develop in PD, affecting almost 50% by 10 years from diagnosis.

However, in some individuals with an alpha-synucleinopathy, significant cognitive problems precede the onset of parkinsonian motor symptoms, and these cases are clinically classified with a diagnosis of Dementia with Lewy Bodies. There is clearly a major degree of overlap between these two conditions both clinically and pathologically, but at present, the clinical distinction rests on the time interval between the onset of motor symptoms and dementia, with a minimum one year interval being required for a diagnosis of PD as opposed to Lewy body dementia (DLB). The SARM NADase inhibitor of the present disclosure an any methods thereof, may be applicable for any type, stage or condition of the PD patient.

In more specific embodiments, the inhibitors identified by the method of the present disclosure may be applicable for PNS. Peripheral Nervous System (PNS) disorders refers to diseases affecting parts of the nervous system outside the brain and spinal cord e.g. the cranial nerves and spinal nerves from their origin to their end. The anterior horn cells, although technically part of the central nervous system (CNS), are sometimes discussed with the peripheral nervous system because they are part of the motor unit. Motor neuron dysfunction results in muscle weakness or paralysis. Sensory neuron dysfunction results in abnormal or lost sensation. Some disorders are progressive and fatal. Peripheral nerve disorders can result from damage to or dysfunction of the one of the following: cell body, myelin sheath, axons, neuromuscular junction. Disorders can be genetic (inherited neuropathies) or acquired (due to toxic, metabolic, traumatic, infectious, or inflammatory conditions such as for example chemotherapy-induced PNS, or diabetic PNS).

Peripheral neuropathies may affect one nerve (mononeuropathy), several discrete nerves (multiple mononeuropathy, or mononeuritis multiplex), multiple nerves diffusely (polyneuropathy), a plexus (plexopathy), a nerve root (radiculopathy). More than one site can be affected e.g. in the most common variant of Guillain-Barre syndrome, multiple segments of cranial nerves, usually the 2 facial nerves, may be affected.

In more specific embodiments, the inhibitors identified by the method of the present disclosure may be applicable for AD. Alzheimer's disease (AD), as used herein refers to a disorder that involves deterioration of memory and other cognitive domains that in general leads to death within 3 to 9 years after diagnosis. The principal risk factor for Alzheimer's disease is age. The incidence of the disease doubles every 5 years after 65 years of age. Up to 5% of people with the disease have early onset AD (also known as younger-onset), that may appear at 40 or 50 years of age. Many molecular lesions have been detected in Alzheimer's disease, but the overarching theme to emerge from the data is that an accumulation of misfolded proteins in the aging brain results in oxidative and inflammatory damage, which in turn leads to energy failure and synaptic dysfunction. More specifically, accumulation of AD within has been shown in structurally damaged mitochondria isolated from the brains of patients with Alzheimer's disease.

Alzheimer's disease may be primarily a disorder of synaptic failure. Hippocampal synapses begin to decline in patients with mild cognitive impairment (a limited cognitive deficit often preceding dementia) in whom remaining synaptic profiles show compensatory increases in size. In mild Alzheimer's disease, there is a reduction of about 25% in the presynaptic vesicle protein synaptophysin. With advancing disease, synapses are disproportionately lost relative to neurons, and this loss is the best correlate with dementia Aging itself causes synaptic loss, which particularly affects the dentate region of the hippocampus.

There is no single linear known chain of events or pathways that could initiate and drive Alzheimer's disease. AD is a progressive disease, where dementia symptoms gradually worsen over a number of years. In its early stages, memory loss is mild, but with late-stage AD, individuals lose the ability to carry on a conversation and respond to their environment. Those with AD live an average of eight years after their symptoms become noticeable to others, but survival can range up to 20 years, depending on age and other health conditions.

The most common early symptom of AD is difficulty remembering newly learned information because AD changes typically begin in the part of the brain that affects learning and memory. As AD advances through the brain it leads to increasingly severe symptoms, including disorientation, mood and behavior changes; deepening confusion about events, time and place: unfounded suspicions about family, friends and professional caregivers; more serious memory loss and behavior changes; and difficulty speaking, swallowing and walking. In some embodiments, the SARM NADase inhibitor of the present disclosure and any methods thereof, may be applicable for any type, stage or condition of the AD patient.

In more specific embodiments, the inhibitors identified by the method of the present disclosure may be applicable for ocular disorders. As used herein, the term “ocular disorder” being associated with axonal and/or cellular degradation/degeneration refers to glaucoma. Glaucoma is a group of eye diseases which result in damage to the optic nerve and cause vision loss. The most common type is open-angle (wide angle, chronic simple) glaucoma, in which the drainage angle for fluid within the eye remains open, with less common types including closed-angle (narrow angle, acute congestive) glaucoma and normal-tension glaucoma. Open-angle glaucoma develops slowly over time and there is no pain. Peripheral vision may begin to decrease, followed by central vision, resulting in blindness if not treated. Closed-angle glaucoma can present gradually or suddenly. The sudden presentation may involve severe eye pain, blurred vision, mid-dilated pupil, redness of the eye, and nausea. Vision loss from glaucoma, once it has occurred, is permanent. Eyes affected by glaucoma are referred to as being glaucomatous.

In more specific embodiments, the inhibitors identified by the method of the present disclosure may be applicable for TBI. A traumatic brain injury (TB), also known as an intracranial injury, is defined as damage to the brain resulting from external mechanical force, such as rapid acceleration or deceleration, impact, blast waves, or penetration by a projectile. Brain function is temporarily or permanently impaired and structural damage may or may not be detectable with current technology. TBI is one of two subsets of acquired brain injury (brain damage that occur after birth); the other subset is non-traumatic brain injury, which does not involve external mechanical force (examples include stroke and infection). All traumatic brain injuries are head injuries, but the latter term may also refer to injury to other parts of the head. However, the terms head injury and brain injury are often used interchangeably. Similarly, brain injuries fall under the classification of central nervous system injuries and neurotrauma.

TBI is usually classified based on severity, anatomical features of the injury, and the mechanism (the causative forces). Mechanism-related classification divides TBI into closed and penetrating head injury. A closed (also called nonpenetrating, or blunt) injury occurs when the brain is not exposed. A penetrating, or open, head injury occurs when an object pierces the skull and breaches the dura mater, the outermost membrane surrounding the brain.

Brain injuries can be classified into mild, moderate, and severe categories. The Glasgow Coma Scale (GCS), the most commonly used system for classifying TBI severity, grades a person's level of consciousness on a scale of 3-15 based on verbal, motor, and eye-opening reactions to stimuli. In general, it is agreed that a TBI with a GCS of 13 or above is mild, 9-12 is moderate, and 8 or below is severe. Similar systems exist for young children. Other classification systems are also used to help determine severity. A current model developed by the Department of Defense and Department of Veterans Affairs uses all three criteria of GCS after resuscitation, duration of post-traumatic amnesia (PTA), and loss of consciousness (LOC). It also has been proposed to use changes that are visible on neuroimaging, such as swelling, focal lesions, or diffuse injury as method of classification. Grading scales also exist to classify the severity of mild TBI, commonly called concussion: these use duration of LOC, PTA, and other concussion symptoms.

In more specific embodiments, the inhibitors identified by the method of the present disclosure may be applicable for MSA. “Multiple system atrophy (MSA)”, is a neuropathology that includes cell loss and gliosis in nigrostriatal and olivopontocerebellar structures taking the form of glial cytoplasmic inclusions containing fibrillar alpha-synuclein within oligodendrocytes. It presents with autonomic dysfunction along with parkinsonism and cerebellar dysfunction in varying combinations and is clinically classified as being either mainly cerebellar in its presentation (MSA-C) or mainly parkinsonian (MSA-P).

Still further, as indicated above, neurodegenerative disorders are clearly associated with vascular and ischemic conditions.

Thus, in some embodiments, the SARM1 inhibitors and uses of the invention may be applicable for any vascular and/or ischemic condition. More specifically, brain ischemia (or cerebral ischemia, cerebrovascular ischemia) is a condition in which there is insufficient blood flow to the brain to meet metabolic demand. This leads to poor oxygen supply or cerebral hypoxia and thus to the death of brain tissue or cerebral infarction/ischemic stroke. It is a sub-type of stroke along with subarachnoid hemorrhage and intracerebral hemorrhage, ischemia leads to alterations in brain metabolism, reduction in metabolic rates, and energy crisis. There are two types of ischemia: focal ischemia, which is confined to a specific region of the brain; and global ischemia, which encompasses wide areas of brain tissue.

In some embodiments, the present disclosure provides inhibitors of SARM1 NADase activity for treatment of neurodegenerative or neurological diseases or disorders that involve axon degeneration or axonopathy. The present disclosure also provides methods of using inhibitors of SARM1 NADase activity to treat, prevent or ameliorate axonal degeneration, axonopathies and neurodegenerative or neurological diseases or disorders that involve axonal and/or cellular degeneration.

In some embodiments, the present disclosure provides SARM1 inhibitors adapted for use in methods of treating neurodegenerative or neurological diseases or disorders related to axonal degeneration, axonal damage, axonopathies, demyelinating diseases, central pontine myelinolysis, nerve injury diseases or disorders, metabolic diseases, mitochondrial diseases, metabolic axonal degeneration, axonal damage resulting from a leukoencephalopathy or a leukodystrophy.

In some further embodiments, said neurodegenerative or neurological disease or disorder may be any one of spinal cord injury, stroke, multiple sclerosis, progressive multifocal leukoencephalopathy, congenital hypomyelination, encephalomyelitis, acute disseminated encephalomyelitis, central pontine myelolysis, osmotic hyponatremia, hypoxic demyelination, ischemic demyelination, adrenoleukodystrophy, Alexander's disease, Niemann-Pick disease, Pelizaeus Merzbacher disease, periventricular leukomalacia, globoid cell leukodystrophy (Krabbe's disease), Wallerian degeneration, optic neuritis, transverse myelitis, amyotrophic lateral sclerosis (ALS, Lou Gehrig's disease), Huntington's disease, Alzheimer's disease, Parkinson's disease, Tay-Sacks disease, Gaucher's disease, Hurler Syndrome, traumatic brain injury, post radiation injury, neurologic complications of chemotherapy (chemotherapy induced neuropathy; CIPN), neuropathy, acute ischemic optic neuropathy, vitamin Bi2 deficiency, isolated vitamin E deficiency syndrome, Bassen-Kornzweig syndrome, Glaucoma, Leber's hereditary optic atrophy. Leber congenital amaurosis, neuromyelitis optics, metachromatic leukodystrophy, acute hemorrhagic leukoencephalitis, trigeminal neuralgia, Bell's palsy, cerebral ischemia, multiple system atrophy, traumatic glaucoma, tropical spastic paraparesis human T-lymphotropic virus 1 (HTLV-1) associated myelopathy, west nile virus encephalopathy, La Crosse virus encephalitis, Bunyavirus encephalitis, pediatric viral encephalitis, essential tremor, Charcot-Marie-Tooth disease, motomeuron disease, spinal muscular atrophy (SMA), hereditary sensory and autonomic neuropathy (HSAN), adrenomyeloneuropathy, progressive supra nuclear palsy (PSP), Fnedrich's ataxia, hereditary ataxias, noise induced hearing loss and congenital hearing loss.

Neuropathies and axonopathies can include any disease or condition involving neurons and/or supporting cells, such as for example, glia, muscle cells or fibroblasts, and, in particular, those diseases or conditions involving axonal damage. Axonal damage can be caused by traumatic injury or by non-mechanical injury due to diseases, conditions, or exposure to toxic molecules or drugs. The result of such damage can be degeneration or dysfunction of the axon and loss of functional neuronal activity. Disease and conditions producing or associated with such axonal damage are among a large number of neuropathic diseases and conditions. Such neuropathies can include peripheral neuropathies, central neuropathies, and combinations thereof. Furthermore, peripheral neuropathic manifestations can be produced by diseases focused primarily in the central nervous systems and central nervous system manifestations can be produced by essentially peripheral or systemic diseases.

Peripheral neuropathies can involve damage to the peripheral nerves and can be caused by diseases of the nerves or as the result of systemic illnesses. Some such diseases can include diabetes, uremia, infectious diseases such as AIDS or leprosy, nutritional deficiencies, vascular or collagen disorders such as atherosclerosis, and autoimmune diseases such as systemic lupus erythematosus, scleroderma, sarcoidosis, rheumatoid arthritis, and polyarteritis nodosa. Peripheral nerve degeneration can also result from traumatic (mechanical) damage to nerves as well as chemical or thermal damage to nerves. Such conditions that injure peripheral nerves include compression or entrapment injuries such as glaucoma, carpal tunnel syndrome, direct trauma, penetrating injuries, contusions, fracture or dislocated bones; pressure involving superficial nerves (ulna, radial, or peroneal) which can result from prolonged use of crutches or staying in one position for too long, or from a tumor; intraneural hemorrhage; ischemia; exposure to cold or radiation or certain medicines or toxic substances such as herbicides or pesticides. In particular, the nerve damage can result from chemical injury due to a cytotoxic anticancer agent such as, for example, taxol, cisplatinin, a proteasome inhibitor, or a vinca alkaloid such as vincristine. Typical symptoms of such peripheral neuropathies include weakness, numbness, paresthesia (abnormal sensations such as burning, tickling, pricking or tingling) and pain in the arms, hands, legs and/or feet. The neuropathy can also be associated with mitochondrial dysfunction.

A peripheral neuropathy can also be a metabolic and endocrine neuropathy which includes a wide spectrum of peripheral nerve disorders associated with systemic diseases of metabolic origin. These diseases include, for example, diabetes mellitus, hypoglycemia, uremia, hypothyroidism, hepatic failure, polycythemia, amyloidosis, acromegaly, porphyria, disorders of lipid/glycolipid metabolism, nutritional/vitamin deficiencies, and mitochondrial disorders, among others. The common hallmark of these diseases is involvement of peripheral nerves by alteration of the structure or function of myelin and axons due to metabolic pathway dysregulation.

Neuropathies can also include optic neuropathies such as glaucoma; retinal ganglion degeneration such as those associated with retinitis pigmentosa and outer retinal neuropathies; optic nerve neuritis and/or degeneration including that associated with multiple sclerosis; traumatic injury to the optic nerve which can include, for example, injury during tumor removal; hereditary optic neuropathies such as Kjer's disease and Leber's hereditary optic neuropathy; ischemic optic neuropathies, such as those secondary to giant cell arteritis; metabolic optic neuropathies such as neurodegenerative diseases including Leber's neuropathy mentioned earlier, nutritional deficiencies such as deficiencies in vitamins B12 or folic acid, and toxicities such as due to ethambutol or cyanide; neuropathies caused by adverse drug reactions and neuropathies caused by vitamin deficiency. Ischemic optic neuropathies also include non-arteritic anterior ischemic optic neuropathy.

Neurodegenerative diseases that are associated with neuropathy or axonopathy in the central nervous system include a variety of diseases. Such diseases include those involving progressive dementia such as, for example, Alzheimer's disease, senile dementia, Pick's disease, and Huntington's disease; central nervous system diseases affecting muscle function such as, for example, Parkinson's disease, motor neuron diseases and progressive ataxias such as amyotrophic lateral sclerosis: demyelinating diseases such as, for example multiple sclerosis; viral encephalitides such as, for example, those caused by enteroviruses, arboviruses, and herpes simplex virus; and prion diseases. Mechanical injuries such as glaucoma or traumatic injuries to the head and spine can also cause nerve injury and degeneration in the brain and spinal cord. In addition, ischemia and stroke as well as conditions such as nutritional deficiency and chemical toxicity such as with chemotherapeutic agents can cause central nervous system neuropathies.

It should be appreciated that the various pathologic disorders disclosed herein are applicable for an aspect of the present disclosure, as discussed herein after.

A further aspect of the present disclosure relates to a kit or system comprising:

(a) at least one near full length catalytically active SARM1 protein or any fragment or variant thereof, or any nucleic acid sequence encoding the SARM1 protein. In some embodiments, the fragment or variant of SARM1 of the kit disclosed herein comprises the TIR domain and the TIR docking site of the ARM-1 domain.

Still further, the kit of the invention may comprise (b), at least one reagent and/or material for quantifying the amount of SARM1 substrate or of any hydrolysis products thereof, and/or for assessing and/or determining the conformation of the SARM1 provided by the kit.

The kit of the invention further comprises (c), at least one SARM1 substrate or any analogues thereof.

In some embodiments, the SARM1 substrate is Nicotinamide adenine dinucleotide+(NAD+), or any analogues thereof. Thus, in some embodiments, the kit or system of the present disclosure comprises: (a) at least one near full length catalytically active SARM1 protein or any fragment or variant thereof, or any nucleic acid sequence encoding the SARM1 protein. In some embodiments, the fragment or variant of SARM1 of the kit disclosed herein comprises the TIR domain and the TIR docking site of the ARM-1 domain.

Still further, the kit of the invention may comprise (b), at least one reagent and/or material for quantifying the amount of NAD+ or of any hydrolysis products thereof, and/or for assessing and/or determining the conformation of the SARM1 provided by the kit.

The kit of the invention further comprises (c), at least one NAD+ or any analogues thereof.

In some embodiments, the full length catalytically active SARM1 comprises an amino acid sequence as denoted by SEQ ID NO: 12, and any variants and derivatives thereof.

In some embodiments, the full length catalytically active SARM1 of the kits if the invention may comprise an amino acid sequence as denoted by SEQ ID NO: 12, and any variants and derivatives thereof.

In some embodiments, the full length active SARM1 is encoded by a nucleic acid molecule comprising a nucleic acid sequence as denoted by SEQ ID NO: 6, and any variants and homologs thereof.

Still further, in some embodiments, the nearly full length catalytically active SARM1 comprises amino acid residues 26 to 724 of the SARM1 of SEQ ID NO: 12. In yet some further embodiments, residues 26 to 724 of the SARM1 comprise the amino acid sequence as denoted by SEQ ID NO. 13, or any variants and derivatives thereof. In some specific embodiments, a nearly full length catalytically active SARM1 useful in the present disclosure comprises the amino acid sequence as denoted by SEQ ID NO. 5, or any variants and derivatives thereof.

In some embodiments the SARM1 protein provided by the kit of the invention may be provided either as soluble polypeptides, or alternatively, attached and/or immobilized either directly or indirectly to at least one solid support.

As described herein, the kits disclosed herein may comprise in some embodiments that the SARM1 protein or any fragments thereof attached or immobilized directly or indirectly on a solid support. As used herein, the term “attached” or “immobilized” refers to a stable association of the protein with a surface of a solid support. By “stable association” is meant a physical association between two entities in which the mean half-life of association is one day or more, two days or more, one week or more, one month or more, including six months or more e.g., under physiological conditions. According to certain embodiments, the stable association arises from a covalent bond between the two entities, a non-covalent bond between the two entities (e.g., an ionic or metallic bond), or other forms of chemical attraction, such as hydrogen bonding, Van der Waals forces, and the like. Solid support suitable for use in the kits of the present invention is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, nanoparticles, polymers, multi-well plates (also referred to as microtiter plates or microplates), membranes, filters, conducting and non-conducting metals, glass (including microscope slides) and magnetic supports. More specific examples of useful solid supports include, silica gels, polymeric membranes such as nitrocellulose, particles, derivatized plastic films, glass beads, cotton, plastic beads, alumina gels, polysaccharides such as Sepharose, nylon, latex bead, magnetic bead, paramagnetic bead, superparamagnetic bead, starch and the like.

In some embodiments, the amount of NAD+ is quantified by the kit of the present disclosure using at least one of, at least one fluorescence assay, at least one HPLC assay and at least one chemiluminescent assay, and wherein said kit or system comprises at least one reagent and/or material required for at least one of said at least one fluorescence assay, at least one HPLC assay and at least one chemiluminescent assay.

Still further, in some embodiments, the fluorescence assay that may be performed by the kit of the present disclosure may be at least one of a Resasurin NAD+ assay and eNAD-based NADase assay.

In some specific embodiments, the fluorescence is measured at 554 nm Excitation and 593 nm emission wave-length. In some further embodiments, the fluorescence is measured at 330 nm Excitation and 405 nm emission wave-length.

In some embodiments, the kit or system of the present disclosure comprises at least one reagent and/or material required for a Resasurin NAD+ assay.

In some embodiments, the NAD+ amount is determined using the kit of the invention by a reverse-phase HPLC. In such case, the kit or system comprises at least one reagent and/or material required for the reverse-phase HPLC.

In some embodiments, for evaluating NAD+, the kit of the invention may provide reagents and means for measuring NAD+ hydrolysis products. In some embodiments, the NAD+ hydrolysis product is ADPR and/or cADPR.

In certain embodiments. SARM1 conformation is determined by cryo-EM. In such case, the kit or system provided by the disclosure comprises at least one reagent and/or material for the cryo-EM analysis.

Still further, in some embodiments, the kit or system further comprises at least one reagent and/or material for a cell viability assay.

In yet some further embodiments, the present disclosure provides a kit for use in a screening method of identifying at least one SARM1 NADase inhibitor.

In more specific embodiments, the kit of the present disclosure may be used for the screening method as disclosed by the invention.

In some embodiments, the kit or systems disclosed herein may further comprise at least one candidate compound. In more specific embodiments, such compound may be at least one of a small molecule, a nucleic acid molecule, an amino acid based molecule, a lipid, a polysaccharides and any combinations thereof.

In certain embodiments, the SARM1 NADase inhibitor identified using the kits and systems of the invention may be competitive or non-competitive inhibitor.

In some embodiments, the SARM1 NADase inhibitor identified using the kits and systems of the invention stabilizes a SARM1 two-ring compact inhibitory conformation by binding at least one of the ARM; TIR-ARM; ARM-ARM; and ARM-SAM inter- and intra-domains interfaces of SARM1.

In yet some specific and non-limiting embodiments, the inhibitor identified by the kit of the invention is an allosteric inhibitor.

Still further, the kit of the present disclosure may further comprise in some embodiments at least one device and/or means required for performing the indicated NAD+ assays and/o the confirmation assays.

In some embodiments, the inhibitor identified by using the kits of the invention does not directly interfere or compete with substrate binding at the TIR domain.

In some embodiments, the inhibitor identified by the kit of the present disclosure is for use in the treatment of conditions or disorders associated directly or indirectly with axonal and/or cellular degradation.

A further aspect of the present disclosure relates to a SARM1 NADase inhibitor obtained by a screening method, or any compositions thereof. In some embodiments, the inhibitor of the invention is identified by a method comprising the steps of:

First (a), incubating (i) at least one candidate compound; and (ii) at least one near full length catalytically active SARM1 or any fragment or variant thereof, or any mixture thereof. In some embodiments, any fragment or variant of SARM1 that may be useful in the methods of the present disclosure comprises the TIR domain and the TIR docking site of the ARM-1 domain.

The next step (b), involves adding to the mixture of (a), at least one SARM1 substrate or any analogues thereof, and incubating the mixture under suitable conditions.

In the next step (c), quantifying the amount of SARM1 substrate or of any hydrolysis products thereof in the incubated mixture of (b); and/or assessing and/or determining the conformation of said SARM1 in the incubated mixture of (b) by a suitable means.

The final step (d), involves identifying the candidate compound as an inhibitor of SARM1 NADase, if step (c) in the presence of the candidate results in at least one of:

(i) if the amount of the SARM1 substrate in the mixture of (c) is not reduced over time;
(ii) if the amount of SARM1 substrate in the mixture of (c) is greater than that of a control mixture that does not contain said candidate compound; and/or (ii) if the SARM1 in the mixture of (c) displays a two-ring compact conformation.

In some embodiments, the SARM1 substrate is Nicotinamide adenine dinucleotide+(NAD+), or any analogues thereof. Thus, in some embodiments, the SARM1 inhibitor of the present disclosure is identified and obtained by a method comprising: first (a), incubating a mixture comprising (i) at least one candidate compound; and (ii) at least one near full length catalytically active SARM1 or any fragment or variant thereof. In some embodiments, any fragment or variant of SARM1 that may be useful in the methods of the present disclosure comprises the TIR domain and the TIR docking site of the ARM-1 domain. The next step (b), involves adding to the mixture of (a), at least one Nicotinamide adenine dinucleotide+(NAD+) or any analogues thereof, and incubating the mixture under suitable conditions. In the next step (c), quantifying the amount of NAD+ or of any hydrolysis products thereof in the incubated mixture of (b); and/or assessing and/or determining the conformation of said SARM1 in the incubated mixture of (b) by a suitable means. The final step (d), involves identifying the candidate compound as an inhibitor of SARM1 NADase, if step (c) in the presence of the candidate results in at least one of: (i) if the amount of said NAD+ in the mixture of (c) is not reduced over time; (ii) if the amount of NAD+ in the mixture of (c) is greater than that of a control mixture that does not contain said candidate compound; and/or (iii) if the SARM1 in the mixture of (c) displays a two-ring compact conformation.

In some embodiments of the SARM1 NADase inhibitor, the amount of the SARM1 substrate, for example, NAD+ is quantified by at least one of, at least one fluorescence assay, at least one HPLC assay, and at least one chemiluminescent assay.

Still further, in certain embodiments, the fluorescence assay used by the methods for identifying the inhibitor of the invention is at least one of a Resasurin NAD+ assay and eNAD-based NADase assay.

In some specific embodiments, the fluorescence is measured at 554 nm Excitation and 593 nm emission wave-length. In some further embodiments, the fluorescence is measured at 330 nm Excitation and 405 nm emission wave-length.

In some embodiments, the NAD+ amount is determined by a Resasurin NAD+ assay.

In yet some further embodiments of the SARM1 NADase inhibitor of the present disclosure, the NAD+ amount is determined by a reverse-phase HPLC.

In some embodiments, the SARM1 NADase inhibitor, the NAD+ hydrolysis product is ADPR and/or cADPR.

In yet some further embodiments of the SARM1 NADase inhibitor, the SARM1 conformation is determined by cryo-EM, in the method for identifying the inhibitor of the invention.

In some further embodiments, of the SARM1 NADase inhibitor disclosed herein, a candidate compound determined as a SARM1 NADase inhibitor is further evaluated by a cell viability assay, and/or an animal model.

Thus, in some embodiments, the SARM1 NADase inhibitor of the present disclosure is further characterized functionally using cell viability assays and in vivo or in vitro animal model for neuronal cell injury, for example, axon degradation assay after axotomy. Thus, in some embodiments, the disclosed SARM1 NADase inhibitor protects from neuronal injury, specifically, protects, attenuates and inhibits axonal degeneration process, in about 10% to about 100%, as compared to non-treated control. More specifically, the inhibitor display reduction and inhibition of axonal degradation in about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, 99.99%, 99.999%, 99.9999%, as compared to non-treated control.

In some embodiments, of the SARM1 NADase inhibitor, the full length catalytically active SARM1 used in the method for identifying the inhibitor comprises an amino acid sequence of residue 26-724 of human SARM1 amino acid sequence.

In some embodiments, the full length catalytically active SARM1 may comprise an amino acid sequence as denoted by SEQ ID NO: 12, and any variants and derivatives thereof.

In some embodiments, the full length active SARM1 is encoded by a nucleic acid molecule comprising a nucleic acid sequence as denoted by SEQ ID NO: 6, and any variants and homologs thereof.

In some embodiments, the SARM1 NADase inhibitor of the present disclosure stabilizes a SARM1 two-ring compact inhibitory conformation by binding at least one of the ARM; TIR-ARM; ARM-ARM; and ARM-SAM inter- and intra-domains interfaces of SARM1.

In yet some further embodiments, the SARM1 NADase inhibitor may be inhibitor is an allosteric inhibitor.

In some embodiments, the inhibitor identified by the method of the invention does not directly interfere or compete with substrate binding at the TIR domain.

Still further, in some embodiments of the SARM1 NADase inhibitor, the candidate compound used for screening and identifying the inhibitor of the present disclosure is at least one of a small molecule, a nucleic acid molecule, an amino acid based molecule, a lipid, a polysaccharides and any combinations thereof.

As indicated above, the SARM1 NADase inhibitor of the present disclosure inhibits SARM NADase activity and therefor protects neuronal cells, increase neuronal cell viability, and decrease axonal degradation. As such, the SARM1 NADase inhibitor of the present disclosure is suitable and therefore applicable and adapted for use in the treatment and prevention of any condition associated with neuronal injury and degradation.

Thus, in some embodiments, the SARM1 NADase inhibitor of the present disclosure may be used in the treatment of conditions or disorders associated directly or indirectly with axonal and/or cellular degradation.

In some further embodiments, the SARM1 NADase inhibitor of the invention may be used for axonal and/or cellular degeneration that is associated with at least one of a neurodegenerative or neurological disorder, axonal damage or injury, axonopathy, a demyelinating disease, a central pontine myelinolysis, a metabolic disease, a mitochondria) disease, metabolic axonal degeneration, axonal damage resulting from a leukoencephalopathy or a leukodystrophy.

Still further, the SARM1 NADase inhibitor of the present disclosure may be applicable for any disorder, for example, any one of amyotrophic lateral sclerosis (ALS), Multiple Sclerosis (MS), Parkinson's disease (PD), Peripheral Nervous System (PNS) disorders, Alzheimer's disease (AD), ocular disorder and traumatic brain injury.

It should be noted that any of the disorders disclosed by the present disclosure herein before in connection with other aspects of the present invention, are also applicable for any of the inhibitors identified by the screening method disclosed herein.

Still further, nonlimiting examples for SARM1 NADase inhibitor of the present disclosure may be any of the compounds identified by the screening methods disclosed herein, for example, any of the TK 138, TK 142 (or any other isothiazolinone derivatives) and TK 174, disclosed by the preset examples.

A further aspect of the present disclosure relates to a composition comprising as an active ingredient an effective amount of at least one SARM1 NADase inhibitor as by the invention, or any nano- or micro-particle, micellar formulation, vehicle or matrix comprising said inhibitor, said composition optionally further comprises at least one of pharmaceutically acceptable carrier/s, excipient/s, auxiliaries, and/or diluent/s.

The compositions of the invention may comprise an effective amount of the SARM1 NADase inhibitor of the invention. The term “effective amount” relates to the amount of an active agent present in a composition, that is needed to provide a desired level of active agent in the bloodstream or at the site of action in an individual to be treated to give an anticipated physiological response when such composition is administered. The precise amount will depend upon numerous factors, e.g., the active agent, the activity of the composition, the delivery device employed, the physical characteristics of the composition, intended patient use (i.e., the number of doses administered per day), patient considerations, and the like, and can readily be determined by one skilled in the art, based upon the information provided herein. An “effective amount” of a the SARM1 NADase inhibitor of the invention can be administered in one administration, or through multiple administrations of an amount that total an effective amount, e.g. within a 24-hour period. It can be determined using standard clinical procedures for determining appropriate amounts and timing of administration. It is understood that the “effective amount” can be the result of empirical and/or individualized (case-by-case) determination on the part of the treating health care professional and/or individual.

Still further, as used herein, an “effective amount” of the SARM1 NADase inhibitor of the present disclosure in the composition o the present invention is meant any amount effective for the protective effect of the disclosed inhibitor on axonal degradation, and/or therapeutic effect on any of the disclosed disorders associated with neuronal and/or axonal degradation as disclosed herein.

The pharmaceutical compositions of the invention can be administered and dosed by the methods of the invention, in accordance with good medical practice, systemically, for example intravenous. It should be noted however that the invention may further encompass additional administration modes. In other examples, the pharmaceutical composition can be introduced to a site by any suitable route including oral, intranasal, or intraocular administration, intraperitoneal, subcutaneous, transcutaneous, topical, intramuscular, intraarticular, subconjunctival, or mucosal.

In yet some further embodiments, the composition of the invention may optionally further comprise at least one of pharmaceutically acceptable carrier/s, excipient/s, additive/s diluents and adjuvant/s.

More specifically, pharmaceutical compositions used to treat subjects in need thereof according to the invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general formulations are prepared by uniformly and intimately bringing into association the active ingredients, specifically, the SARM1 NADase inhibitor of the invention with liquid carriers or finely divided solid carriers or both, and then, if necessary, shaping the product. The compositions may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers. The pharmaceutical compositions of the present invention also include, but are not limited to, emulsions and liposome-containing formulations.

Of particular relevance are formulations of the combinations of the invention are combinations encompassed within a nano- or micro-particles. Nanoscale drug delivery systems using liposomes and nanoparticles are emerging technologies for the rational drug delivery, which offers improved pharmacokinetic properties, controlled and sustained release of drugs and, more importantly, lower systemic toxicity. A particularly desired solution allows for externally triggered release of encapsulated compounds.

More specifically, Controlled drug delivery systems (DDS) have several advantages compared to the traditional forms of drugs. A drug is transported to the place of action, hence, its influence on vital tissues and undesirable side effects can be minimized. Accumulation of therapeutic compounds in the target site increases and, consequently, the required doses of drugs are lower. This modern form of therapy is especially important when there is a discrepancy between the dose or the concentration of a drug and its therapeutic results or toxic effects. Various nanostructures, including liposomes, polymers, dendrimers, silicon or carbon materials, and magnetic nanoparticles are applicable in the present invention. Polymeric nanoparticles are one technology being developed to enable clinically feasible oral delivery.

The term “nanostructure” or “nanoparticle” is used herein to denote any microscopic particle smaller than about 100 nm in diameter. In some other embodiments, the carrier is an organized collection of lipids. When referring to the structure forming lipids, specifically, micellar formulations or liposomes, it is to be understood to mean any biocompatible lipid that can assemble into an organized collection of lipids (organized structure). In some embodiments, the lipid may be natural, semi-synthetic or fully synthetic lipid, as well as electrically neutral, negatively or positively charged lipid. In some embodiments, the lipid may be a naturally occurring phospholipid. Examples of lipids forming glycerophospholipids include, without being limited thereto, glycerophospholipid, phosphatidylglycerols (PG) including dimyristoyl phosphatidylglycerol (DMPG); phosphatidylcholine (PC), including egg yolk phosphatidylcholine, dimyristoyl phosphatidylcholine (DMPC), 1-palmitoyl-2-oleoylphosphatidyl choline (POPC), hydrogenated soy phosphatidylcholine (HSPC), distearoylphosphatidylcholine (DSPC); phosphatidic acid (PA), phosphatidylinositol (PI), phosphatidylserine (PS). Examples of cationic lipids may include, for example, 1,2-dimyristoyl-3-trimethylammonium propane (DMTAP) 1,2-dioleyloxy-3-(trimethylamino) propane (DOTAP); N-[1-(2,3,-ditetradecyloxy)propyl]-N,N-dimethyl-N-hydroxyethylammonium bromide (DMRIE); N-[1-(2,3,-dioleyloxy)propyl]-N,N-dimethyl-N-hydroxy ethyl-ammonium bromide (DORIE); N-[1-(2,3-dioleyloxy) propyl]-N,N,N-trimethylammonium chloride (DOTMA); 3β[N—(N′,N′-dimethylaminoethane) carbamoyl] cholesterol (DC-Chol); and dimethyl-dioctadecylammonium (DDAB), N-[2-[2,5-bis[3-aminopropyl)amino]-1-oxopentyl] amino] ethyl 1-N,N-dimethyl-2,3-bis[(1-oxo-9-octadecenyl)oxy]-1 propanaminium (DOSPA), and ceramide carbamoyl spermine (CCS), or the neutral lipid dioleoylphosphatidyl ethanolamine (DOPE) derivatized with polylysine to form a cationic lipopolymer.

It should be understood that in addition to the ingredients particularly mentioned above, the formulations may also include other agents conventional in the art having regard to the type of formulation in question.

Still further, pharmaceutical preparations are compositions that include one or more targeting cassette present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols.

The active agent may be systemic after administration or may be localized by the use of regional administration, intramural administration, or use of an implant that acts to retain the active dose at the site of implantation. The active agent may be formulated for immediate activity or it may be formulated for sustained release.

Still further, the composition/s of the invention and any components thereof may be applied as a single daily dose or multiple daily doses, preferably, every 1 to 7 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks or even a month. The application of the combination's, composition/s and kit/s of the invention or of any component thereof may last up to a day, two days, three days, four days, five days, six days, a week, two weeks, three weeks, four weeks, a month, two months three months or even more. Specifically, application may last from one day to one month. Most specifically, application may last from one day to 7 days.

In yet a further aspect, the present disclosure provides a method for treating, preventing, inhibiting, reducing, eliminating, protecting or delaying the onset of conditions or disorders associated directly or indirectly with axonal and/or cellular degradation in a subject in need thereof. The method comprises in some embodiments the step of administering to the subject a therapeutically effective amount of at least one SARM1 NADase inhibitor as defined by the present disclosure, or any nano- or micro-particle, micellar formulation, vehicle, matrix or composition comprising the inhibitor.

In more specific embodiments, the inhibitor is obtained by a screening method comprising the steps of (a). incubating (i) at least one candidate compound; and (ii) at least one full length or near full length catalytically active SARM1 or any fragment or variant thereof, or any mixture thereof. The fragment or variant of SARM1 comprises the TIR domain and the TIR docking site of the ARM-1 domain. Th next step (b). adding to the mixture of (a), at least one SARM1 substrate or any analogues thereof, and incubating said mixture under suitable conditions. In the next step (c), quantifying the amount of said SARM1 substrate or of any hydrolysis products thereof in the incubated mixture of (b); and/or assessing and/or determining the conformation of said SARM1 in the incubated mixture of (b) by a suitable means; and (d). identifying said candidate compound as an inhibitor of SARM1 NADase if at least one of:

(i) the amount of said SARM1 substrate in the mixture of (c) is not reduced over time;
(ii) the amount of SARM1 substrate in the mixture of (c) is greater than that of a control mixture that does not contain said candidate compound; and (iii) the SARM1 in the mixture of (c) displays a two-ring compact conformation.

In some embodiments, the SARM1 substrate is Nicotinamide adenine dinucleotide+(NAD+), or any analogues thereof.

A further aspect of the present disclosure relates to a method for treating, preventing, inhibiting, reducing, eliminating, protecting or delaying the onset of conditions or disorders associated directly or indirectly with axonal and/or cellular degradation in a subject in need thereof. The method comprises in some embodiments the step of:

(I) screening for said inhibitor, comprising:
(a). incubating (i) at least one candidate compound; and (ii) at least one full length or near full length catalytically active SARM1 or any fragment or variant thereof, or any mixture thereof, wherein said fragment or variant of SARM1 comprises the TIR domain and the TIR docking site of the ARM-1 domain; (b). adding to the mixture of (a), at least one SARM1 substrate or any analogues thereof, and incubating said mixture under suitable conditions; (c). quantifying the amount of said SARM1 substrate or of any hydrolysis products thereof in the incubated mixture of (b); and/or assessing and/or determining the conformation of said SARM1 in the incubated mixture of (b) by a suitable means; and (d). identifying said candidate compound as an inhibitor of SARM1 NADase if at least one of: (i) the amount of said SARM1 substrate in the mixture of (c) is not reduced over time; (ii) the amount of SARM1 substrate in the mixture of (c) is greater than that of a control mixture that does not contain said candidate compound; and (iii) the SARM1 in the mixture of (c) displays a two-ring compact conformation; and
(II) administering to the subject a therapeutically effective amount of at least one SARM1 NADase inhibitor obtained in step (I), or any nano- or micro-particle, micellar formulation, vehicle, matrix or composition comprising the inhibitor.

In some embodiments, the SARM1 substrate is Nicotinamide adenine dinucleotide+(NAD+), or any analogues thereof.

In some embodiments, axonal and/or cellular degeneration is associated with at least one of a neurodegenerative or neurological disorder, axonal damage or injury, axonopathy, a demyelinating disease, a central pontine myelinolysis, a metabolic disease, a mitochondria) disease, metabolic axonal degeneration, axonal damage resulting from a leukoencephalopathy or a leukodystrophy.

In some embodiments, the methods of the invention may be applicable for any one of amyotrophic lateral sclerosis (ALS), Multiple Sclerosis (MS), Parkinson's disease (PD), Peripheral Nervous System (PNS) disorders, Alzheimer's disease (AD), ocular disorder and traumatic brain injury.

It should be understood that any of the disorders associated directly or indirectly with axonal and/or cellular degradation disclosed in the present disclosure in connection with other aspects of the invention, are also applicable for any of the therapeutic methods disclosed herein.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease.

The term “treatment or prevention” as used herein, refers to the complete range of therapeutically positive effects of administrating to a subject including inhibition, reduction of, alleviation of, and relief from, a condition and illness associated directly or indirectly with axonal and/or synaptic degradation, axonal and/or synaptic degradation symptoms or undesired side effects or axonal and/or synaptic degradation disorders. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction”, “decrease” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%, 100% or more. With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

The term “amelioration” as referred to herein, relates to a decrease in the symptoms, and improvement in a subject's condition brought about by the compositions and methods according to the invention, wherein said improvement may be manifested in the forms of inhibition of pathologic processes associated with the axonal and/or synaptic degradation-related disorders described herein, a significant reduction in their magnitude, or an improvement in a diseased subject physiological state.

The term “inhibit” and all variations of this term is intended to encompass the restriction or prohibition of the progress and exacerbation of pathologic symptoms or a pathologic process progress, said pathologic process symptoms or process are associated with.

The term “eliminate” relates to the substantial eradication or removal of the pathologic symptoms and possibly pathologic etiology, optionally, according to the methods of the invention described herein.

The terms “delay”, “delaying the onset”, “retard” and all variations thereof are intended to encompass the slowing of the progress and/or exacerbation of a disorder associated with the axonal and/or synaptic degradation-related disorders and their symptoms slowing their progress, further exacerbation or development, so as to appear later than in the absence of the treatment according to the invention.

As indicated above, the methods and compositions provided by the present invention may be used for the treatment of a “pathological disorder”, specifically, axonal and/or synaptic degradation-related disorders as specified by the invention, which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health, are used interchangeably and have meanings ascribed to each and all of such terms.

The present invention relates to the treatment of subjects or patients, in need thereof. By “patient” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically, murine subjects. More specifically, the methods of the invention are intended for mammals. By “mammalian subject” is meant any mammal for which the proposed therapy is desired, including human, livestock, equine, canine, and feline subjects, most specifically humans.

A further aspect of the invention relates to a method for inhibiting SARM1 NADase activity in a cell. More specifically, the method comprises the step of contacting the cell with an effective amount of at least one SARM1 NADase inhibitor as defined by the present disclosure, or any nano- or micro-particle, micellar formulation, vehicle, matrix or composition comprising such inhibitor.

The present disclosure further encompasses in additional aspects thereof methods for identifying compounds that inhibit axonal, and/or cellular and/or synaptic degradation. In more specific embodiments, the method may comprise the steps of:

First (a), incubating (i) at least one candidate compound; and (ii) at least one full length, or nearly full length catalytically active SARM1 or any fragment or variant thereof, or any mixture of (i) and (ii). In some embodiments, any fragment or variant of SARM1 that may be useful in the methods of the present disclosure comprises the TIR domain and the TIR docking site of the ARM-1 domain. The next step (b), involves adding to the mixture of (a), at least one SARM1 substrate or any analogues thereof, and incubating the mixture under suitable conditions. In the next step (c), quantifying the amount of SARM1 substrate or of any hydrolysis products thereof in the incubated mixture of (b); and/or assessing and/or determining the conformation of SARM1 in the incubated mixture of (b) by a suitable means.

The next step (d) involves identifying the candidate compound as an inhibitor of SARM1 NADase. More specifically, a candidate compound is identified as an inhibitor of SARM1 NADase if step (c) of the method of the invention, results in at least one of the following outcomes in the presence of the candidate. In some embodiments, a candidate compound is considered as an inhibitor if (i), the amount of the SARM1 substrate in the mixture of (c) is not reduced over time. It should be noted that since NAD+ is SARM substrate, reduction of SARM1 substrate amount over the reaction time reflects SARM1 NADase activity. A candidate compound that attenuates or inhibits the reduction of SARM1 substrate amount, may be considered as inhibiting SARM1 NADase activity. In another alternative or additional embodiment (ii), a candidate compound may be considered as an inhibitor of SARM1 NADase activity, if the amount of SARM1 substrate in the mixture of (c), is greater than that of a control mixture that does not contain the candidate compound. In yet some further additional or alternative embodiment, a candidate compound may be considered as an inhibitor if (iii) if the SARM1 in the mixture of (c) displays a two-ring compact conformation.

The final step is to evaluate the effect of a candidate compound identified in step (d) as an inhibitor of SARM1 NADase, on viability of cells, using a suitable means. In some embodiments, the cells may be neuronal cells. In yet some further embodiments, a compound that inhibit axonal, and/or cellular and/or synaptic degradation, is a compound that leads to increase in cell viability, as compared to cells that were not contacted with such compound.

In some embodiments, the SARM1 substrate is Nicotinamide adenine dinucleotide+(NAD+), or any analogues thereof.

It should be noted that a compound that inhibit axonal, and/or cellular and/or synaptic degradation may be n some embodiments, any of the SARM1 NADase inhibitors disclosed by the invention.

Still further, the present disclosure provides methods for identifying, and screening for compounds for treating disorders or conditions associated directly or indirectly with axonal an/o cellular degeneration.

The present disclosure provides in additional aspects thereof, any of the nucleic acid sequences encoding the nearly full length SARM1, specifically, any nucleic acid sequences comprising sequences encoding the nearly full length SARM1, that may comprise any nucleic acid sequence encoding residues 26 to 724 of SARM1 as denoted by SEQ ID NO. 12. In more specific embodiments, the resent disclosure encompasses any nucleic acid sequence encoding the amino acid sequence of SEQ ID NO. 13, or any derivatives, variants, and homologs thereof. Still further, the present disclosure encompasses any vectors or vehicles comprising these nucleic acid sequences, and any host cells expressing the nucleic acid sequences of the present disclosure.

All definitions, as defined and used herein, should be understood to control over dictionary definitions, definitions in documents incorporated by reference, and/or ordinary meanings of the defined terms.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an.” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one. A, and at least one, optionally including more than one. B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”. “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

### Experimental Procedures

TABLE 1
Key Resources Table
Key Resources Table

| Reagent type | | | | |
|---|---|---|---|---|
| (species) | | Source or | | Additional |
| or resource | Designation | reference | Identifiers | information |
| gene | SARM1 | Imagene | uni prot | |
| (homo sapiens) | | | Q6SZW1 | |
| cell line | HEK293F | Thermo Fisher | Cat# | |
| (homo sapiens) | | Scientific | R79007 | |
| cell line | HEK293T | ATCC | CRL-11268 | |
| (homo sapiens) | | | | |
| gene | SARM1 | Imagene | uni prot | |
| (Zebrafish) | | | F1QWA8 | |
| transfected | pEGFP-N1 | Clontech | | modified |
| construct | | | | |
| (mammalian | | | | |
| vector) | | | | |
| chemical | olyethyieneimine | Polysciences | PEI-MAX | |
| compound, | 40 kDa | | | |
| drug | | | | |
| chemical | Resazurin | SIGMA | R7017 | |
| compound, | sodium salt | | | |
| drug | | | | |
| chemical | NAD+ | SIGMA | NAD | |
| compound, | | | 100-RO | |
| drug | | | | |
| chemical | FMN | SIGMA | F8399 | |
| compound, | | | | |
| drug | | | | |
| chemical | ADH | SIGMA | A3263 | |
| compound, | | | | |
| drug | | | | |
| chemical | Diaphorase | SIGMA | D5540 | |
| compound, | | | | |
| drug | | | | |
| chemical | BSA | ORNAT | B9001S | |
| compound, | | | | |
| drug | | | | |
| chemical | eNAD | SIGMA | N2630 | |
| compound, | | | | |
| drug | | | | |
| chemical | DMSO | SIGMA | 67-68-5 | |
| compound, | | | | |
| drug | | | | |
| chemical | TK138 | MolPort | MolPort- | |
| compound, | | | 023-276-485 | |
| drug | | | | |
| chemical | TK142 | MolPort | Mol Port- | |
| compound, | | | 002-903-305 | |
| drug | | | | |
| chemical | TK174 | MolPort | Mol Port- | |
| compound, | | | 019-885-142 | |
| drug | | | | |
| mice | DRG | Envigo Israel | CD(ICR) | |
| [symbol] Ce1l culture slide | 8-chamber | [symbol] PL Life Sciences | 30108 | |
| chemical | poly-d-lysin | Sigma aldrich | P6407 | |
| compound, | | | | |
| drug | | | | |
| chemical | Laminin | Sigma aldrich | L2020 | |
| compound, | | | | |
| drug | | | | |
| chemical | Neurobasal ™-A | GIBCO | 10888022 | |
| compound, | | | | |
| drug | | | | |
| chemical | B-27 | GIBCO | 17504044 | |
| compound, | | | | |
| drug | | | | |
| chemical | penicillin- | Biological | 03-031-1B | |
| compound, | streptomycin | Industries | | |
| drug | | | | |
| chemical | L-glutamine | Biological | 03-020-1B | |
| compound, | | Industries | | |
| drug | | | | |
| chemical | NGF | Alomone labs | mNGF | |
| compound, | | | 2.5 S N-100 | |
| drug | | | | |
| chemical | [symbol] ormaldehyde 4% | Bio-Lab | 6450323F1 | |
| compound, drug | | | | |
| Antibody | mti-tubulin b III | R&D systems | MAB1195 | |
| | antibody | | | |
| | (Tuj 1 clone) | | | |
| Antibody | Cy3 goat | Jackson | | |
| | anti-mouse | ImmunoResearch | 115-165-146 | |
| | | Laboratories | | |
| microscope | Eclipse Ni-E | Nikon | | |
| | [symbol] right microscope | | | |
| | system | | | |
| software, | MotionCorr2 | (Li et al., 2013) | | |
| algorithm | | | | |
| software, | Gctf | (Zhang, 2016) | | |
| algorithm | | | | |
| software, | Cryosparc V2 | (Punjani et al., | | |
| algorithm | | 2017) | | |
| software, | CIPION wrapper | (Martinez et al., | | |
| algorithm | | 2020) | | |
| software, | Warp | (Tegunov and | | |
| algorithm | | Cramer, 2019) | | |
| software, | CCP4 | [symbol] Winn et al., 2011) | | |
| algorithm | | | | |
| software, | GESAMT | (Krissinel, 2012) | | |
| algorithm | | | | |
| software, | MOLREP | (Vagin and | | |
| algorithm | | [symbol] eplyakov, 2010) | | |
| software, | Coot | (Emsley et al., | | |
| algorithm | | 2010) | | |
| software, | REFMAC5 | [symbol] Murshudov et al.. | | |
| algorithm | | 2011) | | |
| software, | JUGAND | (Lebedev et al.. | | |
| algorithm | | 2012) | | |

[symbol]

indicates data missing or illegible when filed

cDNA Generation and Subcloning.

Cloning of all the constructs was made by PCR amplification from the complete cDNA clone (Imagene) of hSARM1 (uniprot: Q6SZW1). For expression in mammalian cell culture, the near-intact hSARM1w.t. (26ERL . . . GPT724) and the mutants hSARM1E642Q, hSARM1 RR216-7EE, hSARMFP255-6RR, hSARM1RR216-7EE/FP255-6RR and delARM (387SAL . . . GPT724) constructs were ligated into a modified pEGFP-N1 mammalian expression plasmid which is missing the C-terminus GFP fusion protein and includes N-terminal 6*HIS-Tag followed by a TEV digestion sequence. Assembly PCR mutagenesis was used to introduce all the point mutations.

### Protein Expression and Purification.

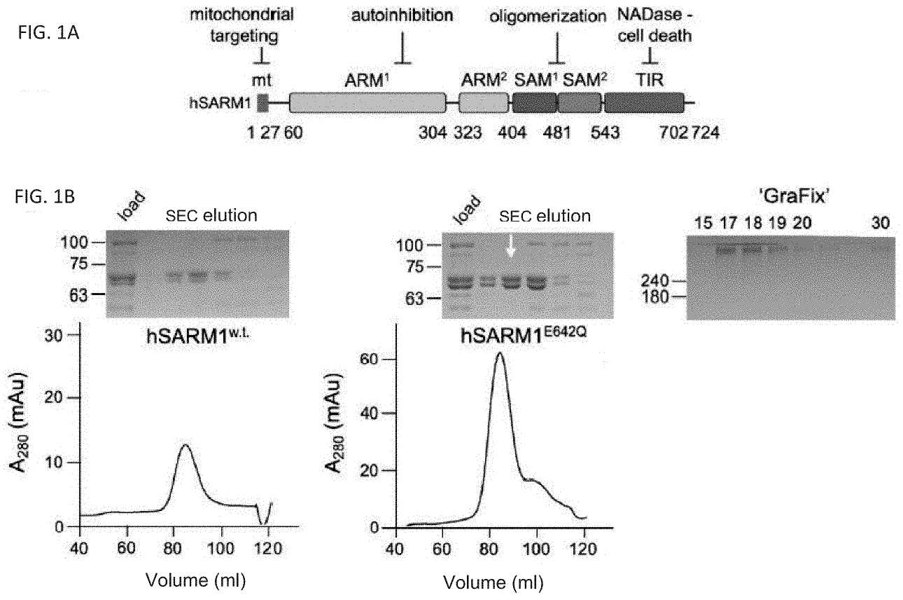

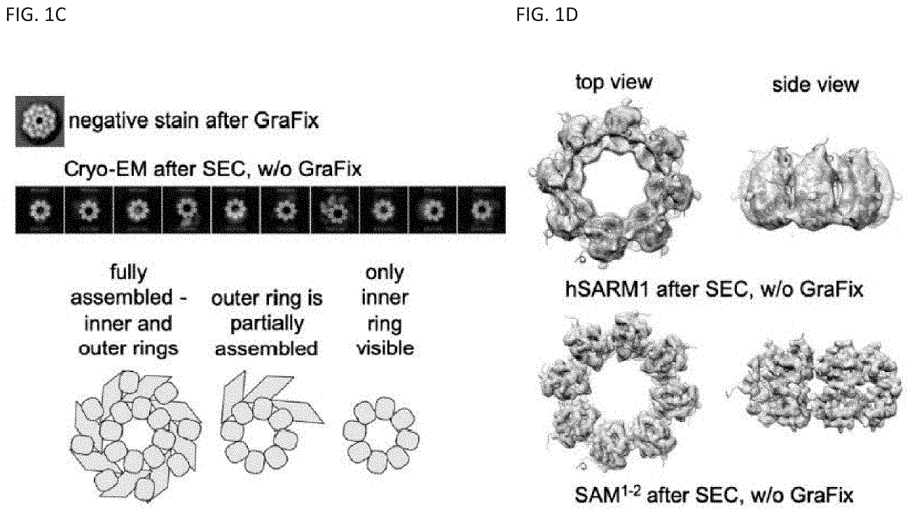

For protein purification, SARM1 w.t, and SARM1E642Q were expressed in HEK293F suspension cell culture, grown in FreeStyle™ 293 medium (GIBCO), at 37° C. and in 8% CO2. Transfection was carried out using preheated (70° C.) 40 kDa polyethyleneimine (PEI-MAX) (Polysciences) at 1 mg of plasmid DNA per 1 liter of culture once cell density has reached 1*106 cells/ml. Cells were harvested 4-(in the case of SARM1w.t.) and 5-(in the case of SARM1E642Q) days after transfection by centrifugation (10 min, 1500×g, 4° C.), re-suspended with buffer A (50 mM Phosphate buffer pH 8, 400 mM NaCl, 5% glycerol, 1 mM DTT, 0.5 mM EDTA, protease inhibitor cocktail from Roche) and lysed using a microfluidizer followed by two cycles of centrifugation (12000×g 20 min). Supernatant was then filtered with 45 μm filter and loaded onto a pre-equilibrated Ni-chelate column. The column was washed with buffer A supplemented with 25 mM Imidazole until a stable baseline was achieved. Elution was then carried out in one step of 175 mM Imidazole, after which protein-containing fractions were pooled and loaded onto pre-equilibrated Superdex 200 HiLoad 16/60 (GE Healthcare) for size exclusion chromatography and elution was performed with 25 mM Phosphate buffer pH 8.5, 120 mM NaCl, 2.5% glycerol, and 1 mM DTT. Protein-containing fractions were pooled and concentrated using a spin concentrator to 1.5 mg/ml. The concentrated proteins were either split into aliquots, flash-frozen in liquid N2 and stored at −80° C. for later cryo-EM visualization and enzymatic assays, or immediately subjected to a ‘GraFix’ (B. Kastner et al., Nat Methods 5, 53-55 (2008)) procedure as follows. Ultracentrifugation was carried out using a SW41Ti rotor at 35,000 rpm for 16 h at 4° C. in a 10-30% glycerol gradient (prepared with gradient Master-ip® BioComp Instruments, Fredericton, Canada), with a parallel 0-0.2% glutaraldehyde gradient, and with buffer composition of 25 mM Phosphate buffer pH 8.5, 120 mM NaCl, and 1 mM DTT. The protein solution volume that was applied to GraFix was 0.4 ml. After ultracentrifugation, the 12 ml gradient tube content was carefully fractionated into 0.75 ml fractions, supplemented with 10 mM aspartate pH 8 to quench crosslinking, using a regular pipette. Most of the cross-linked protein was found at the fractions around 18% glycerol with minor amount at the bottom of the tube (See FIG. 1B). Analysis was made by SDS-PAGE and the dominant 2-3 fractions were pooled and diluted by 25 mM Phosphate buffer pH 8.5, 120 mM NaCl, and 1 mM DTT, so to reach a final glycerol concentration of 2.5%. The diluted sample was then concentrated using a 100 KDa cutoff Centricon® spin concentrator to reach 1 mg/ml protein concentration.

### Cryo-EM Grids Preparation.

Cryo-EM grids were prepared by applying 3 μl protein samples to glow-discharged (PELCO easiGlow™ Ted Pella Inc., at 15 mA for 1 minute) holey carbon grids (Quantifoil R 1.2/1.3, Micro Tools GmbH, Germany). The grids were blotted for 4 seconds and vitrified by rapidly plunging into liquid ethane at −182° C. using Leica EM GP plunger (Leica Microsystems, Vienna, Austria). The frozen grids were stored in liquid nitrogen until the day of cryo-EM data collection.

### Cryo-EM Data Acquisition and Processing.

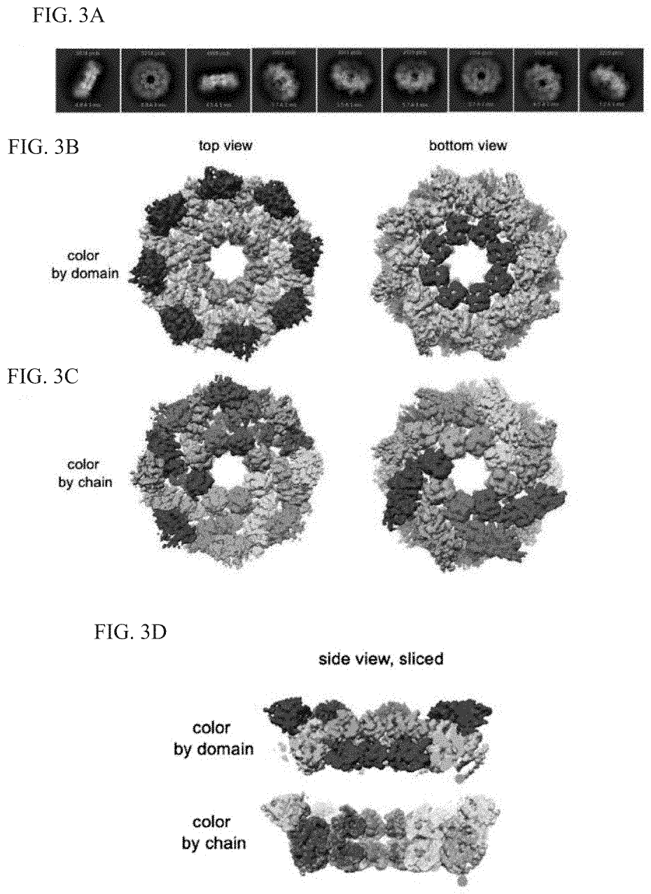

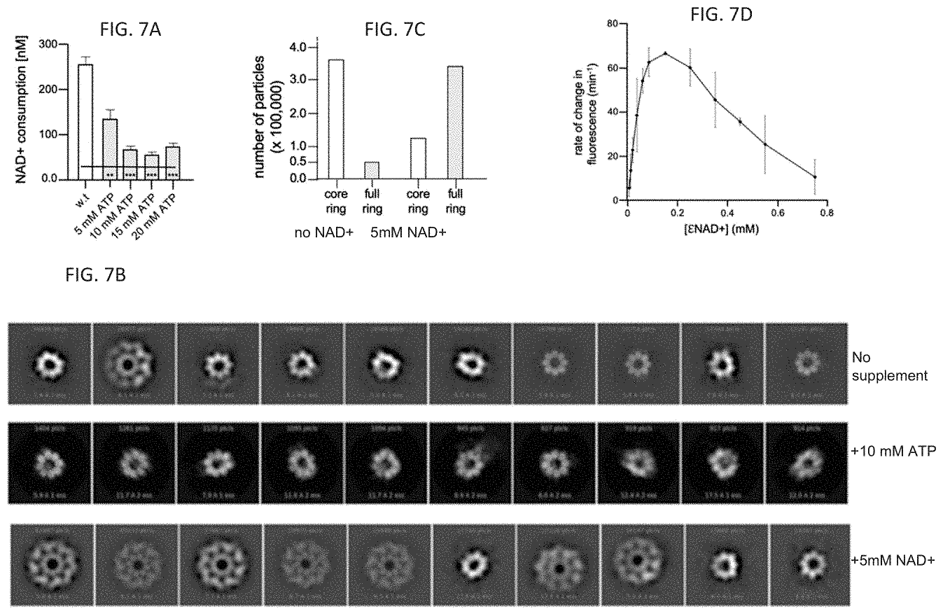

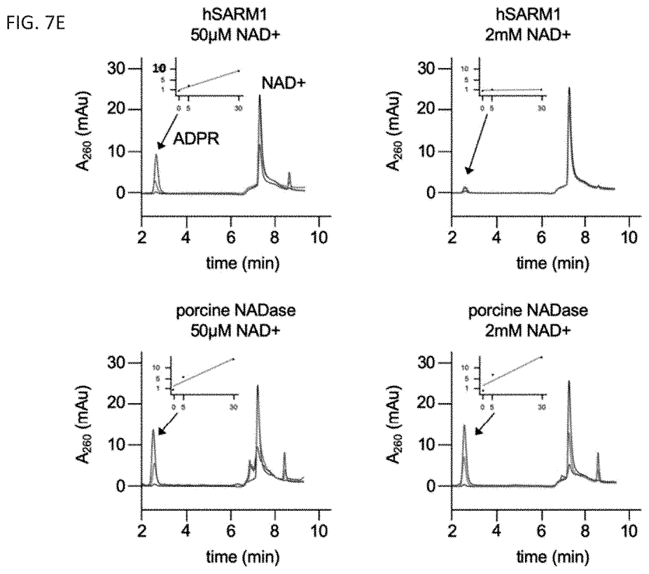

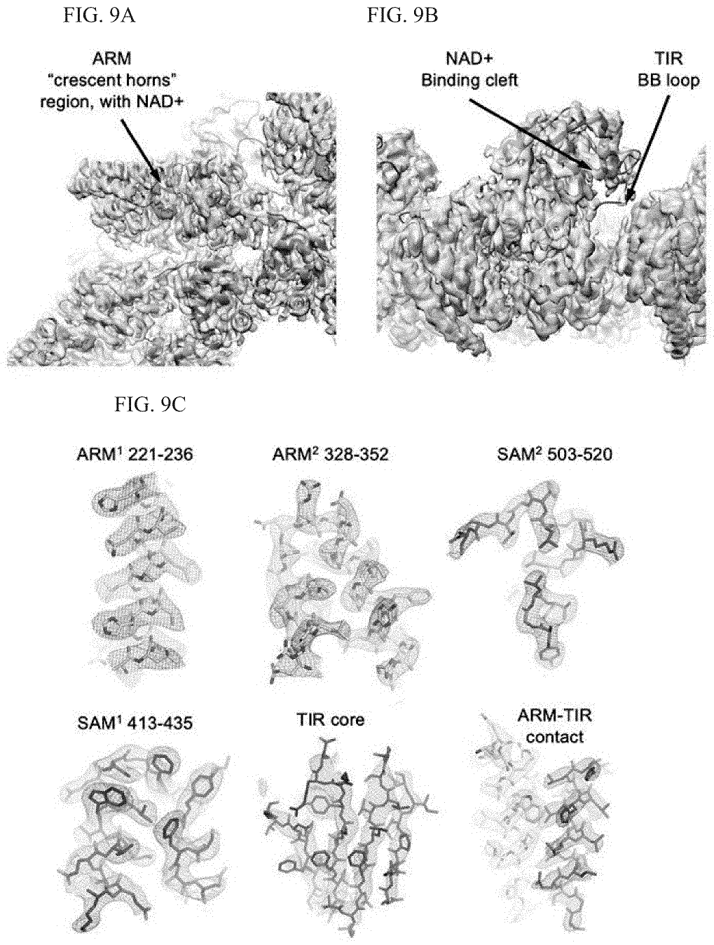

The data presented herein was collected with three separate cryo-electron microscopes: 1) F30 Polara in Ben-Gurion University, Israel was used for all sample preparation optimization. It was also used to collect data sets without and with potential inhibitors (shown in FIG. 1C and the ATP and NMN supplemented classes in FIG. 7B). Finally, it was used for data collection and the 3D reconstructions presented in FIG. 1D. Samples were imaged under low-dose conditions on a FEI Tecnai F30 Polara microscope (FEI, Eindhoven) operating at 300 kV. Datasets were collected using SerialEM (D. N. Mastronarde, et al. Journal of structural biology 152, 36-51 (2005)) on a K2 Summit direct electron detector fitted behind an energy filter (Gatan Quantum GIF) with a calibrated pixel size of 1.1 Å. The energy filter was set to remove electrons >±10 eV from the zero-loss peak energy. The defocus range was set from −1.0 μm to −2.5 μm. The K2 summit camera was operated in counting mode at a dose rate of 8 electrons/pixel/second on the camera. Each movie was dose fractionated into 50 image frames, with total electron dose of 80ē/Å2. Dose-fractionated image stacks were aligned using MotionCorr2 (X. Li et al., Nature methods 10, 584-590 (2013)), and their defocus values estimated by Gctf (K. Zhang, et al. J Struct Biol 193, 1-12 (2016)). The sum of the aligned frames was used for further processing and the rest of the processing was done in Cryosparc V2 (A. Punjani, J. et al. Nature Methods 14, 290-+(2017)). Particles were auto-picked and subjected to local motion correction to correct for beam-induced drift and then 2D classification with 50 classes. The best (based on shape, number of particles and resolution) classes were manually selected containing 5459 (for hSARM1E642Q) and 43868 (for SAM1-2) particles. 1 initial 3D reference was prepared from all particles and 3D refinement imposing C8 symmetry resulted the final map. 2) Titan Krios in ESRF CM01 beamline (E. Kandiah et al., Section D, Structural biology 75, 528-535 (2019)) at Grenoble, France, was used for data collection and 3D reconstruction of the GraFix-ed (FIG. 3, and FIG. 6) and NAD+ supplemented (FIG. 8, FIG. 9A-9C) samples.

Frozen grids were loaded into a 300 kV Titan Krios (ThermoFisher) electron microscope (CM01 beamline at ESRF) equipped with a K2 Summit direct electron-counting camera and a GIF Quantum energy filter (Gatan). Cryo-EM data were acquired with EPU software (FEI) at a nominal magnification of ×165,000, with a pixel size of 0.827 Å. The grid of the GraFix-ed sample was collected in two separate sessions. The movies were acquired for 7 s in counting mode at a flux of 7.06 electrons per Å2 s−1 (data collection 1: 3748 movies) or 6.83 electrons per Å2 s−1 (data collection 2: 4070 movies), giving a total exposure of ˜50 electrons per Å2 and fractioned into 40 frames. 7302 movies of the of the NAD+ supplemented grid sample were acquired for 4 s in counting mode at a flux of 7.415 electrons per Å2 s−1, giving a total exposure of ˜40 electrons per Å2 and fractioned into 40 frames. For each data collection a defocus range from −0.8 μm to ˜2.8 μm was used. Using the SCIPION wrapper (M. Martinez et al., J Chem Inf Model, (2020)) the imported movies were drift-corrected using MotionCor2 and CTF parameters were estimated using Gctf for real-time evaluation. Further data processing was conducted using the cryoSPARC suite. Movies were motion-corrected and contrast transfer functions were fitted. Templates for auto-picking were generated by 2D classification of auto picked particles. For the GraFix-ed data, template-based auto-picking produced a total of 658,575 particles, from which 147,232 were selected based on iterative reference-free 2D classifications for reconstruction of the GraFix-ed structure. In the case of the NAD+ supplemented data, a total of 335,526 particles were initially picked, from which 159,340 were selected based on iterative reference-free 2D classifications for reconstruction. Initial maps of both GraFix-ed and NAD+ supplemented hSARM1 were calculated using Ab-initio reconstruction and high-resolution maps were obtained by imposing C8-symmetry in non-uniform 3D refinement. Working maps were locally filtered based on local resolution estimates. 3) Talos Glacios in EMBL, Grenoble, France was used for data collection and the comparison of NAD+ supplemented and not-supplemented samples (FIG. 7A-7B). Frozen grids were loaded into a 200 kV Talos Glacios (ThermoFisher) electron microscope equipped with a Falcon3 direct electron-counting camera (ThermoFisher). Cryo-EM data were acquired with EPU software (FEI) at a nominal magnification of ×120,000, with a pixel size of 1.224 Å. For the comparative analysis of NAD+ supplement, the grids of +5 mM NAD and no NAD sample were screened and collected. The movies were acquired for 1.99 s in linear mode at a flux of 21.85 electrons per Å2 s−1 (data collection +5 mM NAD: 2408 movies; no NAD: 2439 movies) giving a total exposure of ˜44 electrons per Å2 and fractioned into 40 frames. For each data collection a defocus range from −0.8 μm to −2.8 μm was used. Warp (D. Tegunov, et al. Nat Methods 16, 1146-1152 (2019)) was used for real-time evaluation, for global and local motion correction and estimation of the local defocus. The deep learning model within Warp detected particles sufficiently. Inverted and normalized particles were extracted with a boxsize of 320 pixels. 466135 particles of the +5 mM NAD data, and 414633 particles of the ‘no NAD+’ set were imported into Cryosparc for further processing and subjected to a 2D circular masked classification with 100 classes. The class averages were manually evaluated and designated as either ‘full ring’ or ‘core ring’.

### Cell Viability Assay.

HEK293T cells were seeded onto lysine precoated 24 well plates (100,000 cells in each well) in final volume of 500 μL of DMEM (10% FBS) and incubated overnight in 37° C. under 5% CO2. They were then transfected with different hSARM1 constructs using the calcium phosphate-mediated transfection protocol (R. E. Kingston, et al. Curr Protoc Mol Biol Chapter 9, Unit 9.1 (2003)), with addition of 25 μM Chloroquine (SIGMA) right before the transfection. 6 hours after transfection, the chloroquine-containing DMEM was replaced by fresh complete medium. After 24 hours the medium was removed and replaced with 0.03 mg/ml Resazurin sodium salt (SIGMA) dissolved in Phenol Red free DMEM. All plates were then incubated for 1 h at 37° C. and measured using a SynergyHI (BioTek) plate reader at 560 nm excitation and 590 nm emission wavelengths. All fluorescent emission readings were averaged and normalized by subtracting the Resazurin background (measured in wells without cells) and then divided by the mean fluorescence emission from cells transfected by the empty vector (pCDNA3). HEK293F cells were seeded in 24 well plates (1 million cells in each well) in a final volume of 1 mL of FreeStyle™ 293 medium (GIBCO). The cells were transfected with 1 ug DNA as described before and incubated at 37° C. and in 8% CO2. Live cells were counted using the trypan blue viability assay every 24 hours for three days. Three repeats were performed for each construct.

In Vitro hSARM1 NADase Activity Assays.

### Resasurin Assay:

For quantitation of hSARM1 NADase activity and the inhibitory effect of selected compounds (FIG. 6F; FIG. 7A-7B), purified hSARM1w.t. and hSARM1E642Q proteins were first diluted to 400 nM concentration in 25 mM HEPES pH 7.5, 150 mM NaCl, and then mixed in 25° C. with 1 uM of NAD+(in the same buffer) with a 1:1 v/v ratio. All inhibitors were diluted with the same buffer, and the pH values were measured and if necessary titrated to 7.5. Inhibitors were pre-incubated for 20 min with hSARM1 in 25° C. before mixing with NAD+. At designated time points, reactions were quenched by placing the reaction tubes in 95° C. for 2 min. Measurement of NAD+ concentrations was made by a modified enzymatic coupled cycling assay (K. S. Kanamori et al., NAD. Bio Protoc 8, (2018)). The reaction mix, which includes 100 mM Phosphate buffer pH=8, 0.78% ethanol, 4 uM FMN (Riboflavin 5′-monophosphate sodium salt hydrate), 27.2 U/ml Alcohol dehydrogenase (SIGMA), 1.8 U/ml Diaphorase (SIGMA) and freshly dissolved (in DDW) 8 uM Resasurin (SIGMA), was added to each sample at 1:1 (v/v) ratio and then transferred to 384-well black plate (Corning). Fluorescent data was measured using a SynergyHI (BioTek) plate reader at 554-nm excitation and 593-nm emission wavelengths. Standard curve equation for calculation of NAD+ concentration was created for each assay from constant NAD+ concentrations.

eNAD-Based NADase Assay:

eNAD (Nicotinamide 1,N6-ethenoadenine dinucleotide, SIGMA-N2630) was solubilized in water and mixed with native NAD+ in ration of 1:10 (mol:mol) to a final stock concentration of 10 mM. Serial dilutions were made with 25 mM HEPES pH 7.5, 150 mM NaCl buffer and the final mix was transferred to 384-well black plate (Corning). Reaction started by the addition of hSARM1 to a final concentration 400 nM. Then, eNAD degradation rate was monitored by fluorescence reading (330-nm excitation and 405-nm emission wavelengths) of the plate using a SynergyHI (BioTek) in 25° C. for 3 hours. For each NAD+ concentration, a control reaction without SARM1 was measured and subtracted from the + hSARM1 reading and the slope of the linear area was calculated. For the final plot, average of slopes from 3 separate assays for each concentration was calculated.

### HPLC Analysis:

Purified hSARM1w.t. was first diluted to 800 nM in 25 mM HEPES pH 7.5, 150 mM NaCl, and then mixed in 37° C. with different concentrations of NAD+(in the same buffer) in a 1:1 v/v ratio and incubated for 0, 5 and 30 min. 1:100 (v/v). BSA (NEB Inc. 20 mg/ml) was included, and reactions were stopped by heating at 95° C. for 2 minutes. Where specified, NMN (Sigma-Aldrich-N3501) was added in different concentrations. For control, NAD+ consumption was compared to a commercially available porcine brain NADase 0.025 units/ml (Sigma-Aldrich-N9879).

For NADase activity for screening potential inhibitors, purified SARM1 was first diluted to 800 nM in 25 mM HEPES pH 7.5, 150 mM NaCl, and incubated for 10 minutes in 25° C. with 10 Micro Molar of each inhibitor compound. Then, 50 Micro Molar of NAD+(in the same buffer) in a 1:1 v/v ratio were added and incubated for 30 min in 37° C. 1:100 (v/v) BSA (NEB Inc. 20 mg/ml) was included, and reactions were stopped by heating at 95° C. for 2 minutes.

HPLC measurements were performed using a Merck Hitachi Elite LaChrom HPLC system equipped with an autosampler, UV detector and quaternary pump. HPLC traces were monitored at 260 nm and integrated using EZChrom Elite software. 10 μL of each sample were injected onto a Waters Spherisorb ODS1 C18 RP HPLC Column (5 μm particle size, 4.6 mm×150 mm ID). HPLC solvents are: A: 100% methanol; B: 120 mM sodium phosphate pH 6.0; C double-distilled water (DDW). The column was pre-equilibrated with B:C mixture ratio of 80:20. Chromatography was performed at room temperature with a flow rate of 1.5 ml/min. Each analysis cycle was 12 min long as follows (A:B:C, v/v): fixed 0:80:20 0-4 min; gradient to 20:80:0 4-6 min; fixed 20:80:0 from 6-9 min. gradient to 0:80:20 from 9-10 min; fixed 0:80:20 from 10-12 min. The NAD+ hydrolysis product ADPR was eluted at the isocratic stage of the chromatography while NAD+ elutes in the methanol gradient stage.

Compounds that inhibit at least 50°/o of NADase activity at 10 μM, were further examined for half maximal inhibitory concentration (IC₅₀), where hSARM1′ and zfSARM1^{w.t.} were pre-incubated with doses of inhibitor compounds for 10 min at room temperature-25° C., before the NADase reaction was commenced.

### Calculation of SARM1 Kinetic Parameters.

For Vmax and Km determination, the NADase activity assay was performed with several different NAD+ substrate concentrations and sampled in constant time points. For each NAD+ concentration, linear increase zone was taken for slope (V0) calculation. All data were than fitted to the Michaelis-Menten equation using non-linear curve fit in GraphPad Prism software. Kcat was calculated by dividing the Vmax with protein molar concentration.

### Determination of IC₅₀

Various concentrations of inhibitor compounds were pre-incubated with 0.33 μM hSARM1 or 0.35 μM zfSARM1 for 10 minutes at room temperature, followed by addition of 50 μM NAD. After incubation of 10 minutes in 37° C. the reactions were stopped by heating at 95° C. for 2 minutes. The amount of ADPR product were measured by HPLC and used to calculate the initial rate. IC₅₀ values were calculated by plotting the initial rate to dose of compounds (log 10).

All calculations were performed in GraphPad Prism software.

### Model Building and Refinement.

### GraFix-Ed Map

The monomers of known octameric X-ray structures of the hSARM1 SAM1-2 domains (PDBs 6qwv and 6o0s) were superimposed by the CCP4 (M. D. Winn et al., Acta Cryst. D 67,235-242 (2011)) program GESAMT (E. Krissinel, et al. J Mol Biochem 1, 76-85 (2012)) to identify the conserved regions. The model chosen for MR contained single polypeptide residues A406-A546 from the PDB 6qwv (SAM Model). Superposition of all available structures of the hSARM1 TIR domain (PDBs 6o0q, 6o0r, 6o0u, 6o0qv, and 6o1b) has indicated different conformations of the protein main chain for the BB loop region (amino acid residues 593-607). Two different MR models were prepared for the hSARM1 TIR domain. TIR Model 1 contained regions 562-592 and 608-700 of the high-resolution structure (PDB 6o1b). TIR Model 2 represented assembly of superimposed polyalanine models of all the available hSARM1 TIR domains. Models were positioned into the density map by MR with use of phase information as implemented in program MOLREP (A. Vagin, et al. Acta Cryst. D 66, 22-25 (2010)). The shell scripts of the MOLREP EM tutorial were downloaded from https://www.ccpem.ac.uk/docs.php and adapted to allow simultaneous positioning of eight molecular symmetry related SARM1 copies (MOLREP keyword NCS 800). The search protocol involved Spherically Averaged Phased Translation Function (SAPTF; MOLREP keyword PRFY). The recent version of MOLREP (11.7.02; 29.05.2019) uses modification of the original SAPTF protocol (A. A. Vagin, et al. Acta Crystallogr D Biol Crystallogr 57, 1451-1456 (2001)), adapted for work with EM density maps (Alexey Vagin, private communication). It now performs the Phased RF search step in a bounding box of the search model and not in the whole (pseudo) unit cell. Instead of Phased Translation Function step. MOLREP performs Phased RF search at several points in the vicinity of SAPTF peak and, in addition, applies Packing Function to potential solutions. The SAM Model was positioned into the GraFix-ed density map with a score (Map CC times Packing Function) of 0.753. The positioned SAM Model was used as a fixed model in MOLREP when searching for the TIR domain. The TIR Model 1 was positioned with a score of 0.582. The MR search with the TIR Model 2 gave essentially the same solution with a lower overall score of 0.559, but higher contrast. With both SAM1-2 and TIR domains positions fixed, the MR search for an ideal 10-residue α-helical model allowed location of 64 fragments (8 helices per SARM1 monomer) with scores in the range of 0.78-0.81. These helical fragments were used for building of the ARM domain in Coot (P. Emsley, et al. Acta Cryst. D 66, 486-501 (2010)). The quality of the high resolution GraFix-ed density map was sufficient for assignment of side chains for all ARM domain residues. The TIR domain BB loop region (amino acid residues (a.a) 595-607) was built to fit a relatively poor density map in a conformation different to those observed in X-ray structures. The model was refined using both REFMAC5 (G. N. Murshudov et al.. Acta Cryst. D 67, 355-367 (2011)). Side chains of some Lys residues had blobs of undescribed density attached to them. These were modelled as glutaraldehyde ligands. Geometrical restraints for the di-glutaraldehyde molecule and its links to side chains of Lys residues were prepared using JLIGAND (A. A. Lebedev et al., Acta Crystallogr D Biol Crystallogr 68, 431-440 (2012)). The dictionary file was manually edited to allow links to more than a single lysine residue.

### NAD-Supplemented Map.

Originally, the refined full-length GraFix-ed model was positioned by MR into the NAD-supplemented density map, but differences in the relative positions of the hSARM1 domains were apparent. Therefore. MR search was conducted for separate hSARM1 domains. The SAM Model was positioned with a score of 0.653 into this map. With the fixed SAM Model the TIR Model 1 was found with score of 0.594. With fixed SAM and TIR Models, the ARM domain from the GraFix model was found with score of 0.623. The BB-loop of the TIR domain was built into well-defined electron density map in conformation not observed in any of the X-ray structures and different to that in the GraFix-ed model. A low sigma cutoff map allowed modelling of the loop connecting the SAM and TIR domains. Inspection of the maps indicated NAD+ binding accompanied by structural re-arrangement of the ARM1-ARM2 linker region (a.a. 312-324).

### Accession Numbers.

Coordinates and structure factors have been deposited in the Protein Data Bank with accession numbers 6ZFX, 6ZZ7, 6ZG0, 6ZG1, and in the EMDB with accession numbers 11187, 11586, 11190, 11191 for the GraFix-ed, NAD+ supplemented, not treated, and SAM1-2 models and maps, respectively.

### High-Throughput Inhibitor Screening of Potential Inhibitors

The high-throughput inhibitor-screening assay was preformed using the MultiDrop 384 microplate dispenser (Thermo Scientific) and 1536-well black/black non-binding Microplates (Greiner Bio-One).

The high-throughput inhibitor-screening assay was preformed using the MultiDrop 384 microplate dispenser (Thermo Scientific) and 1536-well black/black non-binding Microplates (Greiner Bio-One). A total of ˜150,000 compounds from the following libraries were screened: LOPAC/SIGMA (Navigator LOPAC¹²⁸⁰) 1280 compounds; MicroSource (Spectrum Collection-Known Drugs (66%), Natural Products (26%) and Other Bioactive Components (8%)) 2400 compounds; Prestwick (Prestwick Chemical Library®-approved drugs) 1200 compounds; New Selleck collection 2020 (Bioactive) 3727 compounds; GSK Inhibitors (GSK Published Kinase Inhibitor Set (PKIS)) 367 compounds; Analyticon (MEGxp: Pure natural compounds from plants) 436 compounds; Analyticon (MEGxm: Pure natural compounds from microorganisms) 469 compounds; Analyticon (MEGxp: Pure natural compounds from plants) 3540 compounds; Selleck Chemicals (Natural Product Library) 144; Enamine (Drug-Like Set (DLS)) 20160 compounds; MayBridge (HitFinder™ Collection) 14400 compounds; ChemDiv (DIVERSet) 100000 compounds; Kinom Set (Kinom Set) 187 compounds; SGC (Epigenetic chemical probes) 97 compounds; FDA cancer library (Anti-cancer FDA approved drugs) 248 compounds.

First, 10 nano-liters of each of the 142000 screened compounds, solubilized in DMSO, were plated using Echo® 550 Liquid Handler (Sunny vale, CA, USA). Then. 5 micro liters of purified SARM1 (at 50 Nano-Molar concentration) in 25 mM HEPES pH 7.5, 150 mM NaCl were added to each well for 10 minutes incubation in 25° C. Next, 2.5 Micro Liters of NAD+(in 0.25 Micro-Molar concentration, in 25 mM HEPES pH 7.5, 150 mM NaCl) were added to each well and incubated for 70 min at 37° C. The compounds concentration at this point were 13.3 Micro Molar.

Measurement of NAD+ concentrations were made by a modified enzymatic coupled cycling assay. 2.5 micro-liters of reaction mix, which includes 100 mM Phosphate buffer pH=8, 0.195% ethanol, 1 micro-Molar FMN (Riboflavin 5′-monophosphate sodium salt hydrate), 6.8 U/ml Alcohol dehydrogenase (SIGMA), 0.45 U/ml Diaphorase (SIGMA), 1.25 mg/ml BSA (SIGMA), 1.25% Tween 20 (SIGMA) and freshly dissolved (in DDW) 2 micro-Molar Resasurin (SIGMA), was added to each well plate and incubated in dark for 1 h to 3 h at 25° C. Fluorescent signals were measured by PheraStar FS plate reader (BMG Labtech, Ortenberg, Germany) at 540-nm excitation and 590-nm emission wavelengths. Data analysis was performed using Genedata software. All values were normalized to positive (no hSARM126-724) and negative (with hSARM126-724) internal controls per plate.

### Small Molecules QC Result Analysis

The identity PASS criteria are when a protonated molecular ion, adduct, or simple fragment are found with relative abundancy >70% e.g., M−H, M+H, M+Na, M+ DMSO, M+H+ ACN, M−H+ formic acid. The purity PASS criteria are when (i) the signal from the Photo Diode Array PDA detector is greater than or equal to 70% of the expected signal, or (ii) ESLD ≥70% with no PDA, or (iii) PDA ≥70% and ELSD ≥70%. The identity FAIL criteria are when the expected mass ions are not found by positive or negative ionization mode using ESI or APCI methods. The purity FAIL criteria are when (i) PDA <70% with no ELSD, or (ii) ELSD <70% with no PDA, or (iii) PDA <70% and ELSD <70%. A sample must meet both identity and purity PASS criteria in order to get a QC Result of PASS. If results are ambiguous; for example, due to a method failure or the absence of the required ionization method, then the sample gets a QC Result of TENTATIVE.

Samples were dissolved in (50 μL, 60 μM) 384-well plates in water:acetonitrile (7:3) trace DMSO; were injected (7 μL) onto an Acquity UPLC® BEH C18 1.7 μm 2.1×50 mm Column at a flow rate of 0.5 mL/min. The mobile phase solvents were as follows: A=0.05% Formic Acid in water:acetonitrile (95:5), B=0.05% Formic Acid in acetonitrile. Elution was achieved via the delivery of a 1 min hold at 100% A 3 min 0-100% B gradient followed by a 1.0 min hold at 100% B prior to re-equilibration.

Instrument Specifications: Waters Acquity UPLC® H class with PDA detector, ELSD and using Acquity UPLC® BEH C18 1.7 μm 2.1×50 mm Column (PN:186002350, SN 02703533825836). MS-system: Waters, SQ detector 2

Axon Degeneration Assay after Axotomy

### Explant Culture

Dorsal root ganglion (DRG) were dissected from E13.5 wild-type mice and plated in 8-wells chamber coated with 10 μg/ml poly-d-lysin (Sigma-Aldrich, St Louis. Mo.) and 10 μg/ml mouse Laminin (Sigma-Aldrich). The DRGs were grown in Neurobasal™-A (NB, GIBCO, 10888022) medium supplemented with B27 (Gibco, Waltham, Mass.), penicillin-streptomycin solution, glutamine (Biological Industries, Beit HaEmek, Israel), and NGF (Alomone Labs. Jerusalem, Israel) in a humidified incubator (37° C. 5% CO2).

### SARM1 Inhibitors Treatment and Axotomy

After 96 h, the medium was exchanged to NGF containing medium, supplemented with 10-30 μM SARM1 inhibitory compounds, which were first dissolved in DMSO to a 10 mM stock concentration. For axotomy, axons were cut using a needle, in close proximity to the cell body and were examined 16 hours post-axotomy.

### Immunohistochemistry

DRG cultures were fixed in 4% formaldehyde (Bio-Lab) and 15% Sucrose in PBS for 1 hr and gently washed 3 times with PBS. Cultures were blocked in blocking solution (3% BSA and 0.1% Triton X-100 in PBS) for 1 hour and then incubated overnight in primary antibody Tuj-1 at 4° C. Samples were gently washed 3 times with PBS and incubated with second antibody goat anti-mouse Cy3 for 1 hour at room temperature and finally washed 3× in PBS.

### Quantification of Axonal Protection and Degeneration

In situ images of DRG explants were taken with Eclipse Ni-E upright Nikon microscope with DS-Qi2 Monochrome cooled digital camera

Images were binarized such that pixels corresponding to axons converted to white, while all other regions converted to black. The images were analyzed using Image) software with algorithm that distinguishes fragmented from intact axonal segments. To each image we calculated the degeneration index (DI) defined as the ratio of fragmented to intact axon number. To quantify axonal protection, we normalized the DI values to DMSO with axotomy, and then we divide one by each calculated value. The data represented as fold change between axotomy with treatments of SARM1 inhibitory compounds to axotomy without treatments. To quantify axonal degeneration caused by the compounds' toxicity, we normalized the DI values to DMSO control without axotomy and generated a degeneration index. From each explant, 4 nonoverlapping frames were randomly collected and quantified per experimental condition.

### Example 1

Definition of the Structure of the Full Length hSARM1
Cryo-EM Visualization of Purified hSARM1

For cryo-EM imaging, the near-intact hSARM1, short of the N′ terminal mitochondria) localization signal (26ERL . . . GPT724) that comprises the amino acid sequence as denoted by SEQ ID NO: 5, and mutated in the NADase catalytic residue E642Q (substitution of the Glu residue to Gln in the respective position 642 of the Wild type SARM1 sequence as denoted by SEQ ID NO. 12), was expressed in mammalian cell culture and isolated to homogeneity using consecutive metal chelate and size exclusion chromatography. hSARM1w.t. (26ERL . . . GPT724) that was not mutated in E642, was also expressed and isolated (FIG. 1B), although with lower yields, and was used in cellular and in vitro activity assays. Cryo-EM images of the purified hSARM1E642Q were first collected. 2D classification (FIG. 1C) and 3D reconstruction (FIG. 1D) revealed an octamer ring assembly with clear depiction of the inner ring, which is attributed to the tandem SAM domains. Only a minor fraction of the particles (˜20%) shows the presence of a partial peripheral ring composed by the ARM and TIR domains. Cryo-EM analysis of the isolated SAM1-2 domains (FIG. 1D), and docking of the crystal structure of the SAM1-2 domains' octamer ring (PDB code 6QWV) into the 3D maps demonstrates that indeed, the ARM and TIR domains are largely missing from this reconstruction, implying a disordered outer ring in ˜80% of the particles. Exploring different buffers, pH and salt conditions, addition of various detergents, as well as variations in cryo-EM grid preparation (e.g. ice thickness) did not affect the visibility of the octamer outer ring considerably. These results are inconsistent with a previous analysis, where low resolution negative stain EM visualization and 2D classification of hSARM1E642Q was used, that showed fully assembled inner and outer ring structures (FIG. 1C) [16]. It was considered, that a gradient fixation (GraFix from B. Kastner et al., Nat Methods 5, 53-55 (2008)) step that involves ultra-centrifugation of the protein sample through a glycerol+ glutaraldehyde cross-linker gradients, which was applied before the negative stain—but not before the cryo-EM sample preparations—might be the cause for the difference between the two measurements. Cryo-EM data collection of GraFix-ed hSARM1E642Q was therefore pursued after dilution of the glycerol from 18% (which severely diminishes protein contrast in cryo-EM) to 2.5%.

2.8-6.5 Å Resolution Structure of a Fully Assembled Compact hSARM1 GraFix-Ed Octamer

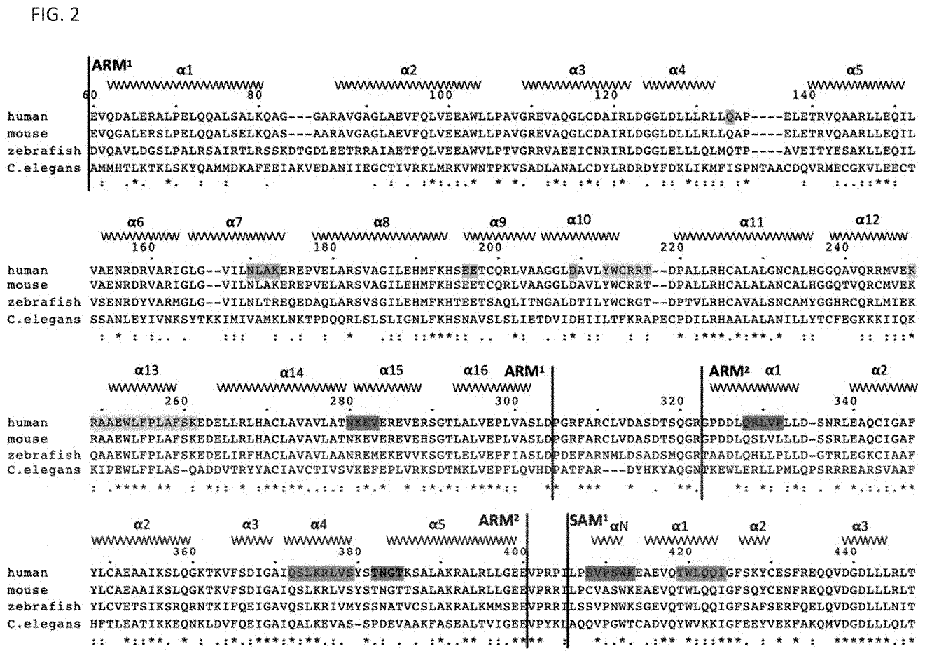

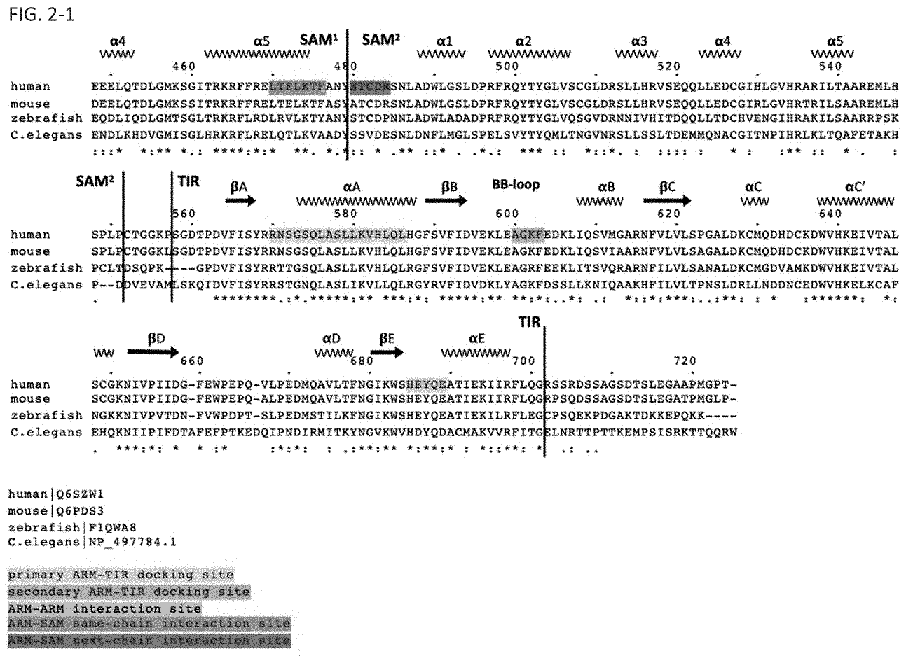

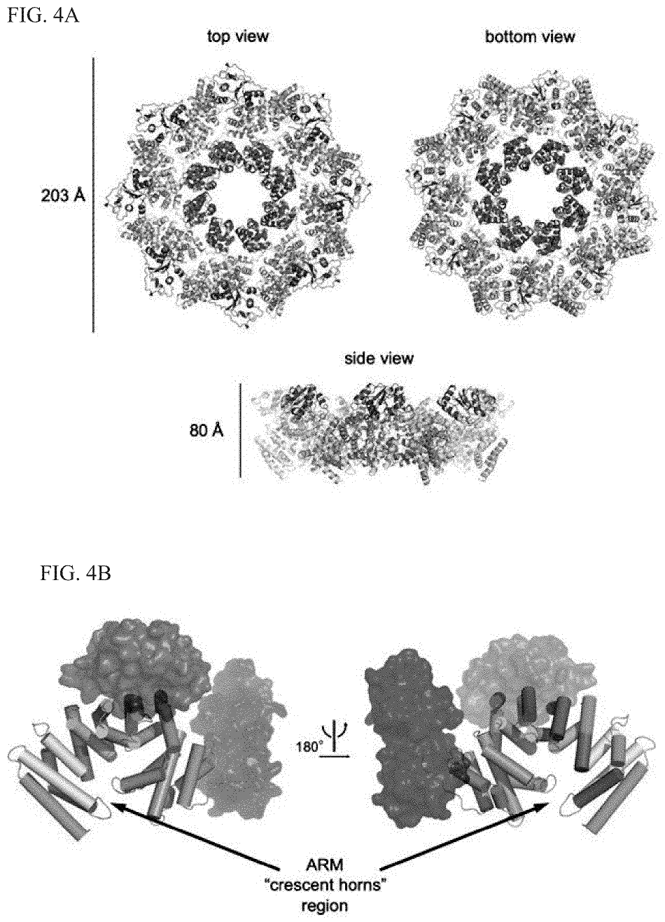

2D classification (FIG. 3A) and 3D reconstruction and refinement (FIG. 3B-3D) of the GraFix-ed hSARM1E642Q were carried out to 2.8-6.5 Å resolution (applying 8-fold symmetry). Overall, the hSARM1E642Q octamer is 203 Å in diameter and 80 Å thick (FIG. 4A). The SAM1-2 domains' inner ring is the best resolved part of the map, to which the high-resolution crystal structure (PDB code 6QWV) was fitted with minute adjustments. The TIR domains are the least defined part of the density map, mostly not revealing side chain positions (FIG. 8A). However, the availability of high-resolution crystal structures of isolated hSARM1 TIR (PDB codes 6O0R, 6O0U) allowed their docking into the well resolved secondary structure elements in the map with very high confidence. The ARM domains show intermediate quality, with well resolved secondary structures and bulky sidechains. This allowed the building of a de-novo atomic model for the entire ARM (FIG. 4A-4B), as no high-resolution structure or homology models of this part of SARM1 are available. The entire atomic model comprises residues 56-700 (see these residues in SEQ ID NOs: 1, 2, 3, 4, of FIG. 2.), with an internal break at the linker, which connects SAM2 to the TIR domain. The structural analyses of the SAM domains and the SAM octamer ring assembly, as well as the atomic details of the TIR domain, are described in previous studies [12, 16]. The cryo-EM structure reveals a closed crescent-shaped ARM region, composed of seven three-helix ARM repeats spanning residues 60-400 (also denoted by SEQ ID NO: 16, FIG. 4B). The ARM topology is split into two interacting parts, designated ARM1 (residues 60 to 303 of the human SARM1 as dented by SEQ ID NO. 1, with five ARM repeats, also denoted by SEQ ID NO: 15) and ARM2 (residues 322 to 400, of the human SARM1 as dented by SEQ ID NO. 1 with two ARM repeats, specifically, also denoted by SEQ ID NO: 17) (FIG. 2, FIG. 4B). The main ARM1-ARM2 interaction interface is hydrophobic and involves helices α14 and 16 of ARM1 and helices α 1, 2 and 3 of ARM2. ARM1 and ARM2 are also interacting at the crescent ‘horns’ through the ARM1 α 2-α 3 loop with the loop that connects ARM1 with ARM2 (residues 305-320 of the human SARM1 as dented by SEQ ID NO. 1, specifically, SEQ ID NO: 18). In the hSARM1 compact octamer, each ARM is directly connected via a linker (residues 400-404 of the human SARM1 as dented by SEQ ID NO. 1, specifically, SEQ ID NO: 19) to a SAM1 domain. Also, each ARM is engaged in several non-covalent interactions with the same-chain SAM and with the clockwise neighboring SAM, when assuming a top view of the structure (FIG. 3B-3D). Although neighboring ARM domains are closely packed, direct interactions between them seem to be limited, engaging a short segment of ARM1 α 9 with the α 4-α 5 loop of ARM2 of the neighboring chain. Additional ARM-ARM interactions are indirect, mediated by the TIR domains. Each TIR binds the ARM ring via two sites, designated the ‘primary’ and ‘secondary’ TIR docking sites (FIG. 6A). The ‘primary’ is larger and engages the TIR helix α A and the EE loop with the ARM1 α 10, α 10-11 loop and α 13. The ‘secondary’ TIR docking site is smaller and involves the TIR BB loop and helix α 7 of the counter-clockwise ARM1 domain (FIG. 2).

### Example 2

The Compact Conformation of hSARM1 is Inhibited for NADase Activity

The GraFix-ed cryo-EM structure revealed a domain organization where the catalytic TIR subunits are separated from each other by their docking onto the ARM peripheral ring, the assembly of which is dependent on the coupling of each TIR with two neighboring ARM domains (FIG. 6A). Since the NADase activity of TIR requires homo-dimerization [11, 13], it was considered that the cryo-EM structure represents an inhibited conformation of SARM1, in which the TIR domains cannot form compact dimers and catabolize NAD+. To test this assumption, amino acid substitutions at the ARM's primary TIR docking site were designed aimed to weaken TIR docking and thus allow their dimerization and subsequent NAD+ catalysis, but without compromising the protein's structural integrity—particularly that of the TIR domain (FIG. 6A). Two pairs of mutations—RR216-7EE of ARM1 helix α10 (substitution of the two Arg residues to two Glu residues in the respective positions 216 and 217 of the Wild type SARM1 sequence as denoted by SEQ ID NO. 12), and FP255-6RR of ARM1 helix α13 (substitution of the Phe and Pro residues to two Arg residues in the respective position 255 and 256 of the Wild type SARM1 sequence as denoted by SEQ ID NO. 12) (FIG. 6C, FIG. 2), were introduced, as well as the double mutant RR216-7EE/FP255-6RR (substitution of the two Arg residues to two Glu residues in positions 216 and 217, and the Phe and Pro residues to two Arg residues in the respective position 255 and 256 of the Wild type SARM1 sequence as denoted by SEQ ID NO. 12), and transiently expressed in HEK-293T cells. Their expression effects on NAD+ levels and cell viability were monitored using a previously demonstrated method, the resazurin fluorescence assay [2, 28]. The results (FIG. 6C) show a rapid decrease in cellular NAD+ levels and 50% cell death 24 hours after transfection of the FP255-6RR and double mutant. This toxicity level is similar to that of a hSARM1 construct missing the entire ARM domain ‘delARM’ (deletion of the respective residues 409 to 724, in the Wild type SARM1 sequence as denoted by SEQ ID NO. 12) (FIG. 6C), which was previously shown to be toxic in neurons and HEK293 cells [2, 16]. This toxic effect was attributed to the removal of auto-inhibitory constraints imposed by the ARM domain. The RR216-7EE mutation has a weaker effect, probably due to the position of these amino acid residues at the margin of the TIR-ARM interface. In conclusion, it was found that hSARM1 inhibition requires ARM-TIR interaction through the ‘primary TIR docking site’. The surface conservation scores of SARM1 orthologs was further calculated. The scores were color coded and plotted on the molecular surface of hSARM1 using the Consurf server (FIG. 6B) (H. Ashkenazy et al., Nucleic Acids Res 44, W344-350 (2016)). The surface-exposed residues reveal a high level of conservation on both the ARM and TIR domains at the binding interface, highlighting the biological importance of the interaction and the possible conservation of its function in auto-inhibition. It was previously suggested, that in order to maintain auto-inhibition, SARM1 is kept as a monomer, that upon activation, undergoes multimeric assembly [30], very much like other apoptotic complexes. The present results show otherwise and explain how hSARM1 avoids premature activation even as a pre-formed octamer, which may allow rapid activation and response.

### Example 3

Isolated hSARM1 is NADase Active In Vitro and Inhibited by Glycerol

As it became clear that the compact two-ring structure is inhibited for NADase activity, it was considered whether the purified hSARM1, that was not subjected to GraFix, and predominantly presents just the inner SAM ring in cryo-EM 2D averaging and 3D reconstruction (FIG. 1C-1D), is active in vitro. Using a resazurin fluorescence assay modified for in vitro application, the rate of NAD+ consumption by hSARM1w.t. was measured in a series of NAD+ concentrations and determined a Km of 28±4 μM, with Vmax of 9±0.3 μM/min (FIG. 6D). This rate was remarkably similar to the previous kinetic study of SARM1, which reported a Km of 24 μM [3]—taking into account, that the previous study used an isolated TIR domain fused to artificial dimerizing and aggregating agents, while the near-intact protein was used here. It was previously demonstrated that nicotinamide mononucleotide (NMN) and a membrane-permeable NMN analogue could activate SARM1 in cultured cells [21-22]. The effect of NMN supplement on the NADase activity of purified hSARM1 was therefore measured and a moderate 30% increase in activity was found with 1 mM NMN (FIG. 6E). These results demonstrate that the purified hSARM1 is indeed already mostly NADase active, even without NMN supplement, consistent with the predominant open conformation seen in cryo-EM (FIG. 1C-1D).

Next, it was examined what confines the GraFix-ed hSARM1 into the compact inhibited conformation (FIG. 3) and measured the in vitro NADase activity in the presence of glycerol (FIG. 6F). It was found that glycerol reduces hSARM1 NADase activity in a concentration-dependent manner, reaching 72% inhibition at 15% glycerol. In the GraFix preparation, hSARM1 was extracted from the gradient tube after it reached approximately 18% glycerol concentration, where SARM1 is at the inhibited compact conformation. It seems likely, that this conformation is maintained after glycerol is removed, due to glutaraldehyde crosslinking which preserves the compact structure.

### Example 4

Unraveling NAD+ Substrate Inhibition Mechanism of hSARM1 and Structure of their Complex
NAD+ Substrate Inhibition of hSARM1

The present results show that hSARM1 NADase activity is suppressed in cell culture, but much less so in vitro after being isolated. The hypothesis was, that in the course of purification from the cytosolic fraction, hSARM1 loses a low-affinity cellular factor, responsible for inhibiting it in the cellular environment. Since SARM1 was previously shown to be activated in cell culture in response to metabolic, toxic and oxidative stresses, it was considered that, perhaps, the hypothesized inhibitory co-factor is a small molecule that is depleted under cellular stress conditions and thus, the inhibition of hSARM1 is released. To follow through on this hypothesis, the impact of several small molecules was tested, which are associated with the cell's energetic state, with two in vitro parameters: hSARM1 NADase activity, and structural conformation (visualized by cryo-EM). It was already established that glycerol meets these two criteria, as it imposes hSARM1 compact conformation (FIG. 3) and reduces NADase activity (FIG. 6F). Glycerol was also found to occupy the active site of the hSARM1 TIR domain in a crystal structure (PDB code 6O0R), directly linking structural data with enzymatic inhibition. However, the glycerol concentrations in which these in vitro experiments were conducted were very high (15-18% for the NADase activity assay and cryo-EM, and 25-30% in the X-ray crystal structure). i.e. 2-4 M, which is considerably higher than the estimated sub 1 mM concentrations of intracellular physiological glycerol (C. C. Li, et al. J Cell Physiol 117, 230-234 (1983)). It was next considered that ATP, being the main energetic compound, with cellular concentrations of 1-10 mM, which are depleted in cell death and axonal degeneration, may alternatively serve as a fit candidate to inhibit hSARM1. Indeed, it was found that ATP inhibits the NADase activity of hSARM1 in a dose-dependent manner (FIG. 7A). However, it did not affect hSARM1 conformation as observed by 2D classification (FIG. 7B). Therefore, it is possible that ATP is a competitive inhibitor of hSARM1, which bind to the TIR domain's active site, and the cryo-EM data further suggest, that a different, allosteric site, is affecting the major changes observed in hSARM1 conformation. It was previously shown that ablation of the cytosolic NAD+ synthesizing enzymes NMNAT1 and 2 decreases cytoplasmic NAD+, and induces SARM1 activation [19, 31]. Therefore, it was postulated that it is the high physiologic concentration of NAD+ itself, that may inhibit hSARM1 through ‘substrate inhibition’ a general mechanism regulating the activity of many enzymes (M. C. Reed, et al. Bioessays 32, 422-429 (2010)).

In cryo-EM visualization, the effect of adding 5-mM NAD+ to hSARM1 was dramatic, showing >80% of the particles in the two-ring, compact conformation—in contrast to the <20% shown in the absence of NAD+(FIG. 7A-7B). To measure NAD+ substrate inhibition of hSARM1, a fluorescent assay was applied using a wide concentrations range of the NAD+ analog, etheno-NAD (eNAD) (mixed 1:10 mol/mol with regular NAD+) that fluoresces upon hydrolysis (ex. λ=330 nm, em. λ=405 nm). The results showed a bell-shape curve (FIG. 7D) with the highest rate of hydrolysis at 100 μM NAD+ and a steady decrease of activity thereafter, in the higher concentrations. In addition, a direct reverse-phase HPLC was used, monitoring of NAD+ consumption by hSARM1 compared with porcine brain NADase. While the rate of hydrolysis was maintained between 50 μM and 2 mM NAD+ by the porcine NADase, hSARM1 was clearly inhibited by the 2 mM NAD+(FIG. 7E).

2.7 Å Resolution Structure of NAD+ Induced hSARM1 Compact Octamer

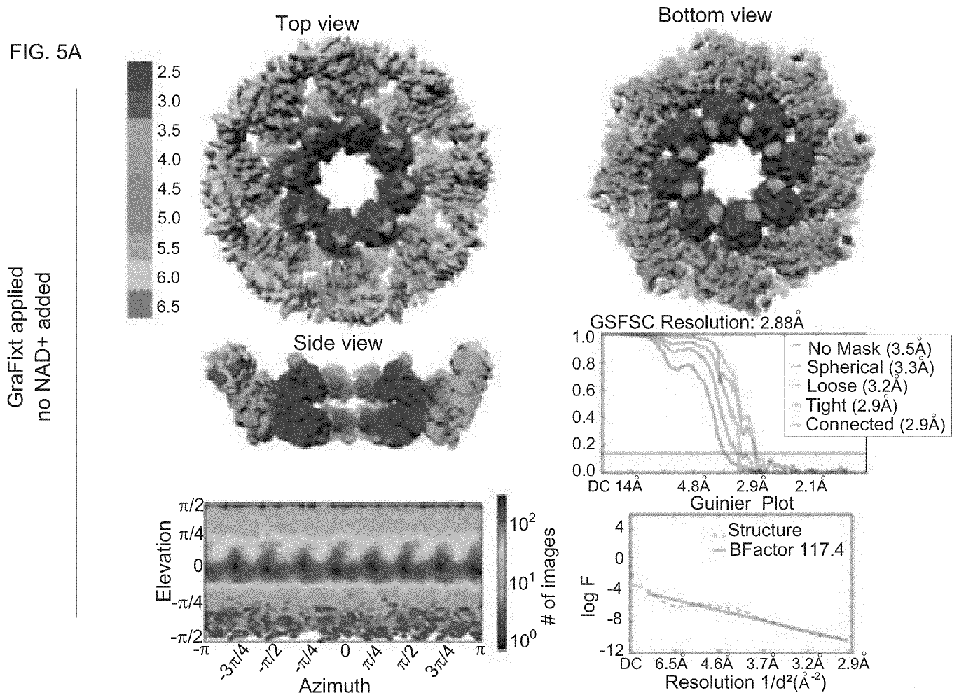

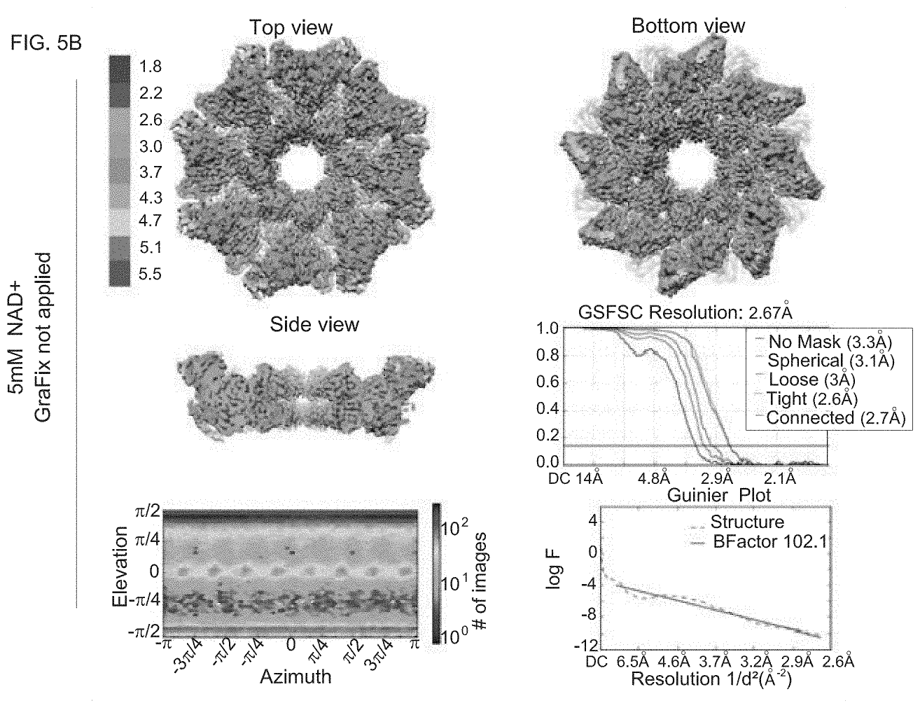

Following the cryo-EM 2D classification (FIG. 7B-7C) and enzymatic (FIG. 7D-7E indications for NAD+ substrate inhibition of hSARM1, a 3D structure determination of hSARM1 complexed with NAD+ at inhibiting concentrations was pursued. For this purpose. 3D reconstruction of hSARM1 supplemented by 5 mM NAD+ was performed after particle picking and 2D classification (FIG. 8A), and was refined to 2.7 Å resolution, with excellent map quality (FIG. 8 and FIG. 9). The structure was largely identical to the GraFix-ed structure, further substantiating the validity of the latter (FIG. 8B). Two small structural differences can be observed between the NAD+ supplemented density map and that of the GraFix-ed hSARM1. The first difference is a 5 Å shift in the position of the distal part of the TIR domain, and the second is a rearrangement of the secondary structure at the region of the ‘crescent horns’, where the tips of ARM1 and ARM2 touch (FIG. 8C, FIG. 9A). In the same region, designated hereafter ‘ARM allosteric site’, an extra-density in the NAD+ supplemented map reveals the binding site of one NAD+ molecule, providing clear atomic details (FIG. 8C, right panel). To probe into the function of the ARM allosteric site, point mutations were introduced in residues 103, 152, 157, 314, 320 and 322 of SARM1 (the respective in the Wild type SARM1 sequence as denoted by SEQ ID NO.12), targeting three structural elements surrounding the NAD+ density. These elements were: ARM1 α2; ARM1 α5-6 loop; and the ARM1-ARM2 loop (FIG. 8C-8D). For control, two other mutations in residues 94 and 363 of SARM1 (the respective in the Wild type SARM1 sequence as denoted by SEQ ID NO.12) were introduced in sites that were not consider to be involved in hSARM1 inhibition or activation. It was hypothesized that NAD+ binding at the ARM allosteric site stabilizes the ARM conformation by interacting with both ARM1 and ARM2, thereby promoting the hSARM1 compact auto-inhibited structure. Therefore, mutations in the allosteric site, that interfere with NAD+ binding, would diminish auto-inhibition and allow hSARM1 activity in cell culture. Indeed, we found that two separate mutations in the ARM1 α5-6 loop (L152A, and R157E, (the respective in the Wild type SARM1 sequence as denoted by SEQ ID NO.12)) and one in the opposite ARM1-ARM2 loop (R322E, (the respective in the Wild type SARM1 sequence as denoted by SEQ ID NO.12)) had a dramatic effect over hSARM1 activity, promoting cell death levels comparable to those induced by the ‘deIARM’ ‘constitutively active’ construct, which is missing the entire ARM domain (FIG. 8D). Two mutations (D314A and Q320 Å, (the respective in the Wild type SARM1 sequence as denoted by SEQ ID NO.12)) that are also located at the ARM1-ARM2 loop but positioned away from the NAD+ molecule, did not induce hSARM1 activation. As expected, the control mutations E94R and K363A (the respective in the Wild type SARM1 sequence as denoted by SEQ ID NO.12) did not affect hSARM1 activity either. Surprisingly, mutating the bulky W103 (the respective in the Wild type SARM1 sequence as denoted by SEQ ID NO.12), which stacks with the NAD+ nicotinamide ring, into an alanine had only a small effect, leading to the notion that W103 does not have a critical role in NAD+ binding. Interestingly, a density that can imply of the presence of an NAD+ molecule at the TIR domain active site was not found, although this site is not occluded by the ARM domain (FIG. 9B). Without being bound by theory, the BB loop, that is interacting with a neighboring ARM domain (FIG. 9B), assumes a conformation that prevents NAD+ entry into the binding cleft. Another conspicuous distinction between the two maps, is the difference in domain-based heterogeneity in map quality in the GraFix-ed structure with respect to the relative homogeneity in the NAD+ supplemented structure (FIG. 5A, 5B). While the SAM and (to a lesser extent) ARM regions are well-resolved in the GraFix-ed density map, the TIR domain is not very clear. Without being bound by theory, a likely reason for the difference in map homogeneity is that while the compact arrangement in the GraFix-ed structure is artificially imposed by high glycerol concentration, the NAD+ supplement seems to induce a more natural compact folding.

FIG. 10 illustrates a model for hSARM1 inhibition and activation. In homeostasis, the cellular NAD+ concentration is high enough and binds to an allosteric site that drives hSARM1 compact conformation. In this conformation, the catalytic TIR domains (red) are docked on ARM domains (yellow) apart from each other, unable to form close dimers required for NAD+ catalysis. When cellular NAD+ levels drop as a result of reduced NAD+ synthesis (e.g. inhibition of NMNAT1/2) or increased NAD+ consumption, the inhibiting NAD+ molecules fall off hSARM1, leading to the disintegration of the ARM-TIR outer ring assembly. Still held by the constitutively-assembled SAM inner ring, the now-released TIR domains are at a high local concentration that facilitates their dimerization and ensued NADase activity. When released from allosteric inhibition, hSARM1 is only subjected to competitive inhibition such as by its products ADPR and NAM, which are not found in high enough concentrations to block its activity entirely. This leads to an almost complete consumption of the NAD+ cellular pool and to an energetic catastrophe from which there may be no return.

### Example 5

### In Vitro Activity Assays for Screening SARM1 Inhibitors

As described above, using in-vitro enzymatic assays, cryo-EM, and experiments in cultured cells, it was found that SARM1 NADase activity is allosterically inhibited by the tight packing of ARM and catalytic TIR domains around an inner SAM domain ring and that this tight inhibitory packing is stabilized by the binding of NAD+ molecules to a site at the ARM domains, which is distal to the catalytic site of the TIR domains. Furthermore, it was found that, in the absence of NAD+ supplement, purified near-intact SARM1 (uniport: Q6SZW1, 26ERL . . . GPT724) is largely not inhibited (due to loss of bound cellular NAD+ in the purification process) and has NADase activity that can be measured using the Resasurin and HPLC-based in-vitro assays.

Based on these results, it was concluded that pharmacological SARM1 inhibition could be achieved not only by compounds that directly interfere or compete for substrate binding at the TIR domain, but also by chemical compounds that stabilize the full-length protein's inhibitory conformation by binding at any of the ARM; TIR-ARM; ARM-ARM; and ARM-SAM inter- and intra-domains interfaces.

that the inventors therefore developed in vitro activity assays that measure the NADase activity of a purified near-intact SARM1 and not just that of an active TIR domain.

For initial high-throughput screening, a modified form of the Resasurin NAD+ assay, specially designed for high-throughput inhibitor screen, is used. The resulting potential compound hits picked by the high-throughput Resasurin assay, are further validated by a HPLC-based assay (see details in Experimental procedures). More specifically, the high-throughput inhibitor-screening assay was preformed using the 384 microplate dispenser and 1536-well Microplates. First, 10 nano-liters of each of the 142000 screened compounds, were plated, each in a separate well, and 5 micro-liters of purified SARM1 (at 50 Nano-Molar concentration) were added to each well for 10 minutes incubation in 25° C. Next, 2.5 Micro Liters of NAD+(in 0.25 Micro-Molar concentration, in 25 mM HEPES pH 7.5, 150 mM NaCl) were added to each well and incubated for 70 min at 37° C. Measurement of NAD+ concentrations were made by a modified enzymatic coupled cycling assay as described in the experimental procedures. The resulting potential compound hits picked by the high-throughput Resasurin assay, are further validated by a HPLC-based assay. Compounds showing stable levels of NAD+ over time, or NAD+/ADPR ration that is greater than 1, ae further evaluated for their effect on cell viability using the procedures disclosed in the experimental procedures.

### Example 6

### Optimizing the High Throughput Screen for SARM1 Inhibitors and Reciprocal HPLC Assay

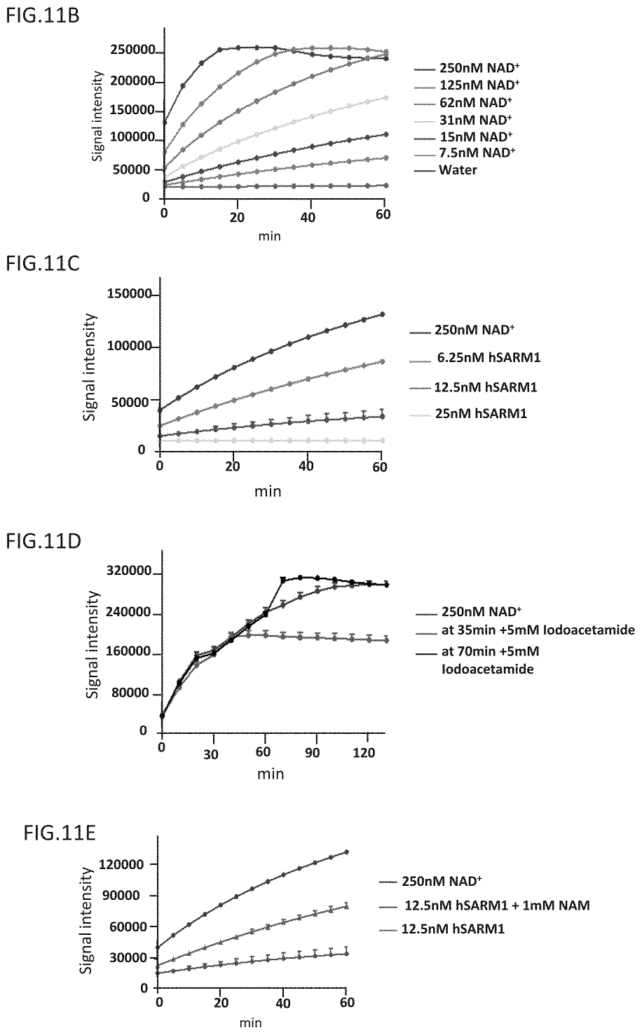

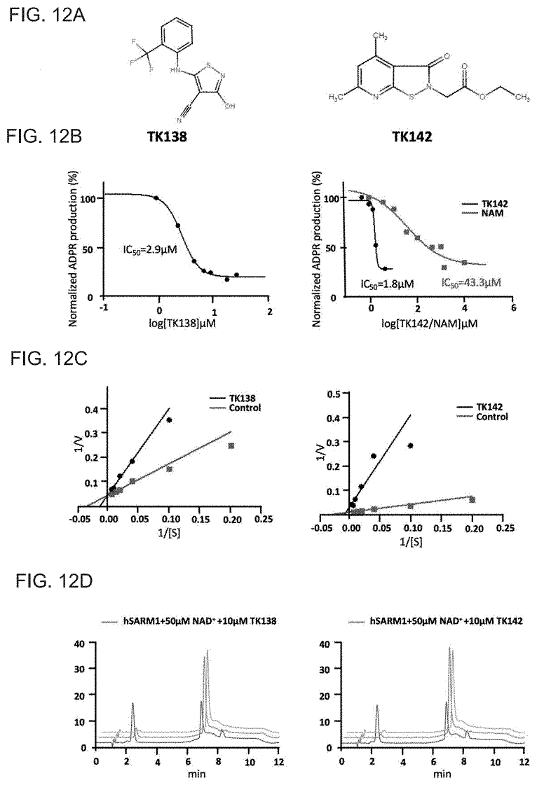

To discover new and effective inhibitors for hSARM1, we have applied a modified fluorescence assay for high throughput screening. The adaptations introduced in this assay aim to achieve: stable fluorescent signal over time, good sensitivity for inhibition by screened compounds, high signal-to-noise, good reproducibility, accuracy, and diminishing of false-positive readouts. The assay is based on the resazurin in vitro application, as reported in (Spomy et al., Structural basis for SARM1 inhibition and activation under energetic stress. Elife 9 (2020), and measures NAD+ hydrolysis by isolated hSARM1. The basic principle of the assay is that the coupled activities of alcohol dehydrogenase and diaphorase, which convert resazurin to the fluorescent resorufin, depend on the amount of available NAD+. Pre-incubation of the NAD+ component with active hSARM1 NADase, reduces the amount of NAD+ and by that—of the output fluorescent signal. Introducing a hSARM1 inhibitory compound in the hSARM1-NAD+ incubation stage, will result in less hSARM1 NADase activity, more available NAD+ for the alcohol dehydrogenase and diaphorase coupled reaction, and more output florescence (FIG. 11A). Undesired inhibition of either alcohol dehydrogenase or diaphorase by the screened compounds can not result in more florescence, and therefore, does not produce false positive signals. To establish a working protocol for high throughput screening, the inventors looked for optimum hSARM1 and starting NAD+ concentrations that would allow to confidently identify as little as 20% inhibitory activity by the screened compounds. It was found that hSARM1 at 12.5 nM and NAD+ at a range of 0-250 nM gives the best performance. In this way, the fluorescence signal trendlines do not overlap at any timepoint for over an hour, allowing to distinguish between small differences in NAD+ concentrations (FIGS. 11B and 11C). Stopping the further development of the fluorescence reaction by the addition of the dehydrogenase inhibitor iodoacetamide keeps the signal stable for at least another hour (FIG. 11D). For control, 1 mM nicotinamide (NAM), that proved to inhibit SARM1 NADase activity in a dose-response manner, was used (FIG. 11E) (Essuman et al., The SARM1 Toll/Interleukin-1 Receptor Domain Possesses Intrinsic NAD(+) Cleavage Activity that Promotes Pathological Axonal Degeneration. Neuron 93, 1334-1343 e1335 (2017)). It should be noted that NAM inhibition of SARM1 was recently put to question (Angeletti et al., 2021), suggesting that instead of preventing NAD+ catalysis it participates in a base exchange reaction, but for the practical purpose of the present disclosure, to measure available NAD+ level, it makes no difference. After establishing the working conditions and protocol of the assay, it was applied for high throughput screening in the Nancy and Stephen Grand Israel National Center for Personalized Medicine. About 150,000 compounds were screened from commercially available libraries of small molecular weight compounds. Out of the 120 molecules that showed at least 30% reduction in fluorescent signal, 31 molecules were selected, based on pharmacokinetics, drug-likeness and medicinal chemistry properties (Daina et al., SwissADME: a free web tool to evaluate pharmacokinetics, drug-likeness and medicinal chemistry friendliness of small molecules. Sci Rep 7, 42717 (2017)). These elected molecules were validated for inhibition with a reciprocal HPLC assay (FIG. 12D), which includes 10 μM of the inhibitor compound, 50 μM of NAD+ and 250 nM of hSARM1. Under these conditions, several compounds that were also re-validated by mass spectroscopy analysis, inhibited ≥50% of NAD+ hydrolysis activity, compared to control. These representative compounds were further analyzed for in vitro SARM1 inhibition (IC₅₀ and Lineweaver Burk plots) and inhibition of ex viva mouse DRG assay for Wallerian degeneration after axotomy.

### Example 7

### In Vitro Inhibitors Characterization—IC₅₀, Lineweaver-Burk Plots, and Species Specificity

At that point, the analysis was focused on the following representative SARM1 inhibitory compounds (designated 110, and presented in SMILES format):

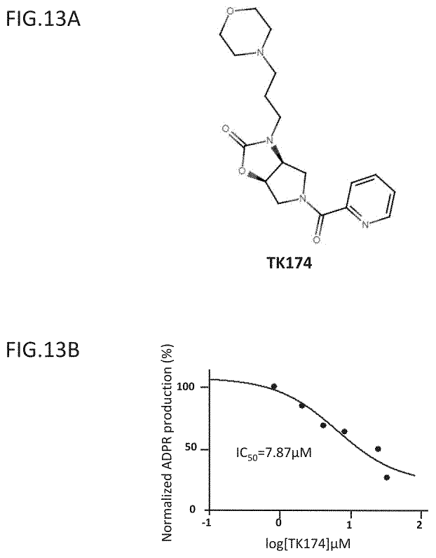

TK138: [H]OCl═NSC(N([H])C2=CC═C2C(F)(F)F)=CIC #N, also denoted by Formula, as shown by FIG. 12A:
TK142: CCOC(═O)CN1SC2=NC(C)═CC(C)=C2Cl═O, also denoted by Formula II, as shown by FIG. 12A;
TK174: O═C(N1C[C@H]2OC(═O)N(CCCN3CCOCC3)[C@H]2Cl)c4ccccn4, also denoted by Formula III, as shown by FIG. 13A.

NADase in vitro analysis of hSARM1 in the presence of 50 μM NAD+ showed that these seven compounds have IC₅₀≤ 10 μM (TK 138, 142, and 174) (FIG. 12A, 12B, and FIG. 13A, 13B). Control experiment with the known SARM inhibitor NAM (nicotinamide) showed IC₅₀=43.3 μM (FIG. 12B), consistent with previously reported inhibitory values (Essuman et al., 2017). The inhibitory mode of these compounds (TK 138, 142) was next characterized, and the NADase activity was measured at different NAD+ concentrations (1-160 μM) with and without the presence of 10 μM of the five inhibitors. As seen in the Lineweaver-Burk plot, TK 138, 142, showed a competitive pattern (FIG. 12C).

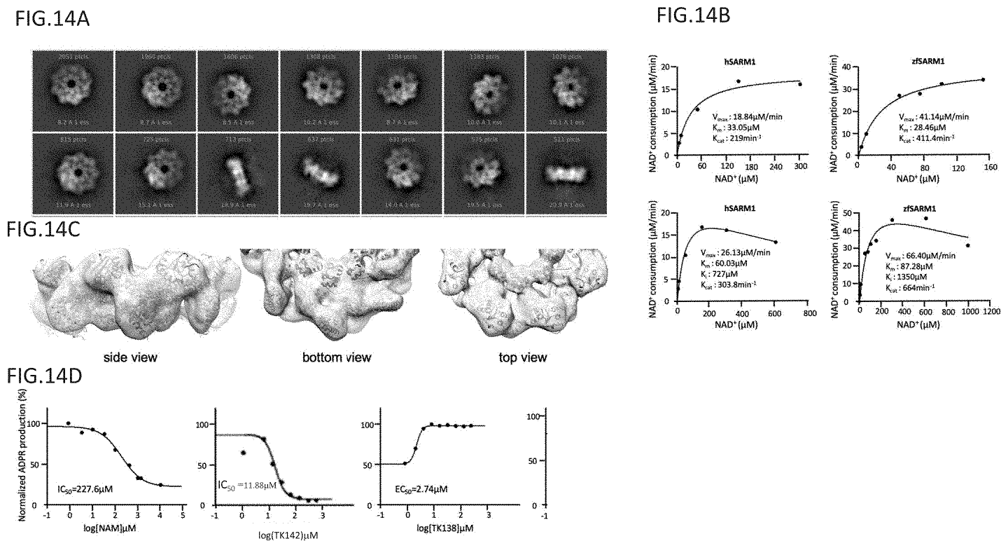

To further characterize these inhibitors, their species specificity was examined, and their effect on isolated zebrafish SARM1 (zfSARM1) as compared to hSARM1, was measured. First, the NADase activity was measured and the 3D structure of purified zfSARM1 (FIG. 14A-14C) was determined. The inventors found that the structure of zfSARM1 is very similar to that of hSARM1, with the latter's atomic model docks very well into the former's 3D cryo-EM reconstruction map (FIGS. 14A and 14B). Next, NADase kinetic behavior of zfSARM1 was measured and was found to be similar to that of hSARM1, with Km and Kcat values of 33 μM and 219 min⁻¹ for hSARM1 and 28 μM and 411 min⁻¹ for zfSARM1 (FIG. 14C). The inventors also found similar apparent inhibition by substrate (as Ki for NAD+) and by product (as IC₅₀ for NAM) in zfSARM1 (Ki=1350 μM; IC₅₀=227 μM) as are in hSARM1 (Ki=727 μM; IC₅₀=43.3 μM). Taken together, the inventors have established that there is a high level of similarity in structure, regulation, and enzymatic activity between the human and zebrafish SARM1. However, it was found that the responses of the zebrafish and human SARM1 towards the TK inhibitors, are in most cases inconsistent (FIG. 14D). The TK 142 compound, an Isothiazolinone derivative, shows specific inhibitory effect on zfSARM1, albite less clear at low inhibitor concentrations. On the contrary, the TK138 compound has a specific activating effect, bringing up zfSARM1 NADase activity with EC₅₀ (half-maximal effective concentration) at 2.7 μM.

### Example 8

### Axon Degeneration Assay—DRG Axotomy

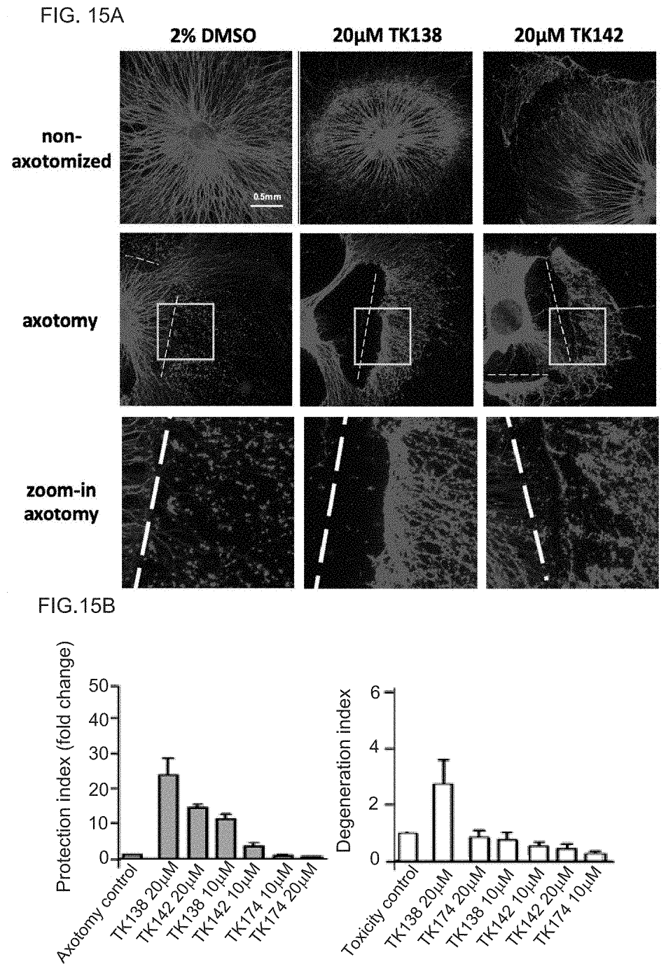

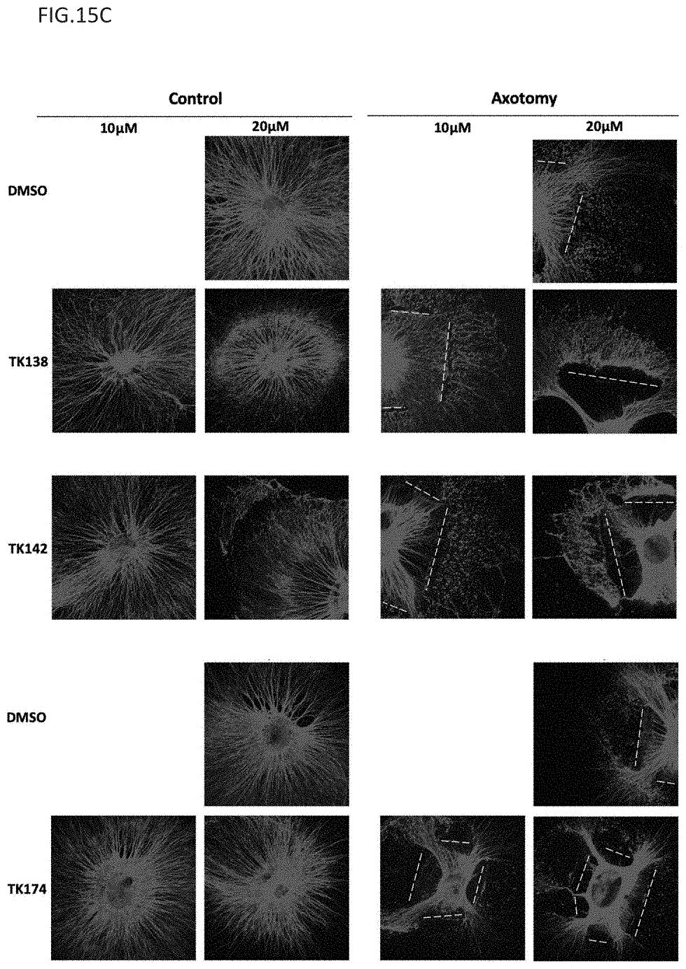

The selected inhibitors were next tested in the in vitro axonal degeneration assay (Shacham-Silverberg et al., Phosphatidylserine is a marker for axonal debris engulfment but its exposure can be decoupled from degeneration. Cell Death Dis 9, 1116 (2018)). In this assay, E13.5 mouse DRGs are plated and left to grow axons for 96 hours before severing of the axons proximally to the DRG ganglion. While the severed axons from SARM1−/− mouse remain intact, as previously shown in (Osterloh et al., 2012), w.t DRG axons get fragmented and are eliminated almost completely 16 hours post axotomy. To test if the newly identified hSARM1 inhibitors can also protect axons from Wallerian degeneration, the inventors supplemented the DRGs medium with 10-30 μM of hSARM1 inhibitory compounds (TK 138, 142, 174) right before axotomy. To evaluate toxic side effects, 10-30 μM of the inhibitory compounds were applied to DRG explants, without performing axotomy. Overall, toxicity was manifested as the thinning, and in some cases elimination, of axons' extremities, at the growth cones region. However, other toxic effects could also be observed, such as the elimination of the segments of axons that are proximal to the DRG ganglion. Mild manifestations of such toxic effects were observed for almost all tested compounds, usually, in a concentration-depended manner. The results are presented in FIGS. 15A, 15B, 15C. The most effective compounds in providing high level of protection, while being only very mildly toxic is TK138. TK142 is not toxic at all but provides only mild protection. Similarly, TK174 did not protect axons after axotomy and was not toxic. It is possible, that the reason why TK174 and TK142 are neither toxic nor protective, is due to impermeability of these compounds into the neurons' cytosol.

1. A screening method for identifying a sterile a and HEAT/armadillo motif-containing protein-1 (SARM1) NADase modulator, the method comprising:
(a). incubating a mixture comprising (i) at least one candidate compound; and (ii) at least one full length or near full length catalytically active SARM1 or any fragment or variant thereof, wherein said fragment or variant of SARM1 comprises the TIR domain and the TIR docking site of the ARM-1 domain;
(b). adding to the mixture of (a), at least one SARM1 substrate or any analogues thereof, and incubating said mixture under suitable conditions;
(c). quantifying the amount of said SARM1 substrate or of any hydrolysis products thereof in the incubated mixture of (b); and/or assessing and/or determining the conformation of said SARM1 in the incubated mixture of (b) by a suitable means;
(d). identifying said candidate compound as an inhibitor of SARM1 NADase if at least one of:
(i) the amount of said SARM1 substrate in the mixture of (c) is not reduced over time;
(ii) the amount of SARM1 substrate in the mixture of (c) is greater than that of a control mixture that does not contain said candidate compound; and
(ii) the SARM1 in the mixture of (c) displays a two-ring compact conformation.

2. The method screening according to claim 1, wherein said SARM1 substrate is Nicotinamide adenine dinucleotide+(NAD+) or any analogues thereof.

3. The method screening according to claim 2, wherein said amount of NAD+ is quantified by at least one of, at least one fluorescence assay, at least one HPLC assay and at least one chemiluminescent assay.

4. The method screening according to claim 3, wherein at least one of:
(i) said fluorescence assay is at least one of a Resasurin NAD+ assay and eNAD-based NADase assay; and
(ii) said NAD+ amount is determined by a reverse-phase HPLC.

5. The screening method according to claim 4, wherein said NAD+ amount is determined by a Resasurin NAD+ assay.

6. The screening method according to claim 1, wherein said NAD+ hydrolysis product is adenosine diphosphoribose (ADPR) and/or cyclic ADP Ribose (cADPR).

7. The screening method according to claim 1, wherein said SARM1 conformation is determined by cryo-electron microscope (cryo-EM).

8. The screening method according to claim 1, wherein a candidate compound determined as a SARM1 NADase inhibitor is further evaluated by at least one of: (i) a cell viability assay; and (ii) assay performed using an animal model.

9. The screening method according to claim 1, wherein said full length catalytically active SARM1 comprises an amino acid sequence as denoted by SEQ ID NO: 12, and any variants and derivatives thereof, and wherein the near full length catalytically active SARM1 comprises an amino acid sequence as denoted by any one of SEQ ID NO. 13, SEQ ID NO. 5, and any variants and derivatives thereof.

10. The screening method according to claim 1, wherein said SARM1 NADase inhibitor is at least one of:
(i) an inhibitor that stabilizes a SARM1 two-ring compact inhibitory conformation by binding at least one of the ARM; TIR-ARM; ARM-ARM; and ARM-SAM inter- and intra-domains interfaces of SARM1; and
(ii) an inhibitor that is an allosteric inhibitor.

11. The screening method according to claim 1, wherein said candidate compound is at least one of a small molecule, a nucleic acid molecule, an amino acid based molecule, a lipid, a polysaccharides and any combinations thereof.

12. The screening method according to claim 1, wherein said inhibitor is adapted for use in the treatment of conditions or disorders associated directly or indirectly with axonal degradation.

13. The screening method according to claim 12, wherein said axonal and/or cellular degeneration is associated with at least one of a neurodegenerative or neurological disorder, axonal damage or injury, axonopathy, a demyelinating disease, a central pontine myelinolysis, a metabolic disease, a mitochondrial disease, metabolic axonal degeneration, axonal damage resulting from a leukoencephalopathy or a leukodystrophy, and wherein said disorder is any one of amyotrophic lateral sclerosis (ALS), Multiple Sclerosis (MS), Parkinson's disease (PD), Peripheral Nervous System (PNS) disorders, Alzheimer's disease (AD), ocular disorder and traumatic brain injury.

14. A SARM1 NADase inhibitor or any composition or any nano- or micro-particle, micellar formulation, vehicle or matrix comprising said inhibitor, wherein said inhibitor is obtained by a screening method comprising the steps of:
(a). incubating a mixture comprising (i) at least one candidate compound; and (ii) at least one full length or near full length catalytically active SARM1 or any fragment or variant thereof, wherein said fragment or variant of SARM1 comprises the TIR domain and the TIR docking site of the ARM-1 domain;
(b). adding to the mixture of (a), at least one SARM1 substrate or any analogues thereof, and incubating said mixture under suitable conditions;
(c). quantifying the amount of said SARM1 substrate or of any hydrolysis products thereof in the incubated mixture of (b); and/or assessing and/or determining the conformation of said SARM1 in the incubated mixture of (b) by a suitable means;
(d). identifying said candidate compound as an inhibitor of SARM1 NADase if at least one of:
(i) the amount of said SARM1 substrate in the mixture of (c) is not reduced over time;
(ii) the amount of SARM1 substrate in the mixture of (c) is greater than that of a control mixture that does not contain said candidate compound; and
(ii) the SARM1 in the mixture of (c) displays a two-ring compact conformation.

15. The SARM1 NADase inhibitor according to claim 14, wherein said inhibitor is at least one of:
(i) an inhibitor that stabilizes a SARM1 two-ring compact inhibitory conformation by binding at least one of the ARM; TIR-ARM; ARM-ARM; and ARM-SAM inter- and intra-domains interfaces of SARM1; and (ii) an inhibitor that is an allosteric inhibitor.

16. A method for treating, preventing, inhibiting, reducing, eliminating, protecting or delaying the onset of conditions or disorders associated directly or indirectly with axonal and/or cellular degradation in a subject in need thereof, the method comprising the step of administering to said subject a therapeutically effective amount of at least one SARM1 NADase inhibitor or any nano- or micro-particle, micellar formulation, vehicle, matrix or composition comprising said inhibitor, wherein said inhibitor is obtained by a screening method comprising the steps of:
(a). incubating (i) at least one candidate compound; and (ii) at least one full length or near full length catalytically active SARM1 or any fragment or variant thereof, or any mixture thereof, wherein said fragment or variant of SARM1 comprises the TIR domain and the TIR docking site of the ARM-1 domain;
(b). adding to the mixture of (a), at least one SARM1 substrate or any analogues thereof, and incubating said mixture under suitable conditions;
(c). quantifying the amount of said SARM1 substrate or of any hydrolysis products thereof in the incubated mixture of (b); and/or assessing and/or determining the conformation of said SARM1 in the incubated mixture of (b) by a suitable means;
(d). identifying said candidate compound as an inhibitor of SARM1 NADase if at least one of:
(i) the amount of said SARM1 substrate in the mixture of (c) is not reduced over time;
(ii) the amount of SARM1 substrate in the mixture of (c) is greater than that of a control mixture that does not contain said candidate compound; and
(ii) the SARM1 in the mixture of (c) displays a two-ring compact conformation.

17. The method according to claim 16, for treating, preventing, inhibiting, reducing, eliminating, protecting or delaying the onset of conditions or disorders associated directly or indirectly with axonal and/or cellular degradation in a subject in need thereof, the method comprising the step of:
(I) screening for said inhibitor, comprising:
(a). incubating (i) at least one candidate compound; and (ii) at least one full length or near full length catalytically active SARM1 or any fragment or variant thereof, or any mixture thereof, wherein said fragment or variant of SARM1 comprises the TIR domain and the TIR docking site of the ARM-1 domain;
(b). adding to the mixture of (a), at least one SARM1 substrate or any analogues thereof, and incubating said mixture under suitable conditions;
(c). quantifying the amount of said SARM1 substrate or of any hydrolysis products thereof in the incubated mixture of (b); and/or assessing and/or determining the conformation of said SARM1 in the incubated mixture of (b) by a suitable means;
(d). identifying said candidate compound as an inhibitor of SARM1 NADase if at least one of:
(i) the amount of said SARM1 substrate in the mixture of (c) is not reduced over time;
(ii) the amount of SARM1 substrate in the mixture of (c) is greater than that of a control mixture that does not contain said candidate compound; and
(iii) the SARM1 in the mixture of (c) displays a two-ring compact conformation; and
(II) administering to said subject a therapeutically effective amount of at least one SARM1 NADase inhibitor obtained in step (I), or any nano- or micro-particle, micellar formulation, vehicle, matrix or composition comprising said inhibitor.

18. The method according to claim 16, wherein said axonal and/or cellular degeneration is associated with at least one of a neurodegenerative or neurological disorder, axonal damage or injury, axonopathy, a demyelinating disease, a central pontine myelinolysis, a metabolic disease, a mitochondrial disease, metabolic axonal degeneration, axonal damage resulting from a leukoencephalopathy or a leukodystrophy.

19. The method according to claim 18, wherein said disorder is any one of amyotrophic lateral sclerosis (ALS), Multiple Sclerosis (MS), Parkinson's disease (PD), Peripheral Nervous System (PNS) disorders, Alzheimer's disease (AD), ocular disorder and traumatic brain injury.

20. A method for inhibiting SARM1 NADase activity in a cell, the method comprising the step of contacting said cell with an effective amount of at least one SARM1 NADase inhibitor, or any nano- or micro-particle, micellar formulation, vehicle, matrix or composition comprising said inhibitor, wherein said inhibitor is obtained by a screening method comprising the steps of:
(a). incubating (i) at least one candidate compound; and (ii) at least one full length or near full length catalytically active SARM1 or any fragment or variant thereof, or any mixture thereof, wherein said fragment or variant of SARM1 comprises the TIR domain and the TIR docking site of the ARM-1 domain;
(b). adding to the mixture of (a), at least one SARM1 substrate or any analogues thereof, and incubating said mixture under suitable conditions;
(c). quantifying the amount of said SARM1 substrate or of any hydrolysis products thereof in the incubated mixture of (b); and/or assessing and/or determining the conformation of said SARM1 in the incubated mixture of (b) by a suitable means;
(d). identifying said candidate compound as an inhibitor of SARM1 NADase if at least one of:
(i) the amount of said SARM1 substrate in the mixture of (c) is not reduced over time;
(ii) the amount of SARM1 substrate in the mixture of (c) is greater than that of a control mixture that does not contain said candidate compound; and
(ii) the SARM1 in the mixture of (c) displays a two-ring compact conformation.
